(19) United States
(12) Patent Application Publication
(10) Pub. No.: US 2022/0348589 A1
(43) Pub. Date: Nov. 3, 2022

(54) Diazaspiro Compound and Use Thereof

(71) Applicants: SK Innovation Co., Ltd., Seoul (KR); SK ie technology Co., Ltd., Seoul (KR)

(72) Inventors: Cheol Woo Kim, Daejeon (KR); Hyo Shin Kwak, Daejeon (KR); So Young Lee, Daejeon (KR)

(21) Appl. No.: 17/723,794

(22) Filed: Apr. 19, 2022

(30) Foreign Application Priority Data
Apr. 20, 2021 (KR) 10-2021-0050891

(51) Int. Cl. C07D 487/10; C08G 73/10; C08J 5/18

(52) U.S. Cl. CPC C07D 487/10; C08G 73/1085; C08J 5/18; C08J 2379/08

(57) ABSTRACT

The present invention relates to a diazaspiro compound and a use thereof. The diazaspiro compound may be significantly advantageously used as a monomer for producing a polyimide film having excellent optical properties.

### CROSS-REFERENCE TO RELATED APPLICATION

This application claims priority to Korean Patent Application No. 10-2021-0050891 filed Apr. 20, 2021, the disclosure of which is hereby incorporated by reference in its entirety.

### BACKGROUND OF THE INVENTION

### Field of the Invention

The following disclosure relates to a diazaspiro compound and a use thereof, and more particularly, to a diazaspiro compound and a use thereof as a monomer advantageously used for producing a polyimide film.

### Description of Related Art

Polyimide (PI) has attracted attention as a material that has high heat resistance and is light and flexible. Polyimide is a polymer having a relatively low crystallinity or mostly non-crystalline structure, which has an advantage that it is easy to synthesize, it may be used to form a thin film, and it does not require a cross-linking agent for curing. Also, polyimide is a polymeric material that has excellent heat resistance and chemical resistance, excellent mechanical properties, electrical properties, and dimensional stability due to its transparency and rigid chain structure. Therefore, polyimide has been widely used as electrical and electronic materials for automobiles, aerospace, flexible circuit boards, liquid crystal alignment films for liquid crystal displays (LCDs), adhesives, and coating agents.

Meanwhile, a flexible device is manufactured by applying a polyimide precursor composition onto a transport substrate, curing the polyimide precursor composition to form a film, completing a device through a subsequent process such as deposition of a thin film transistor (TFT) and an organic film, and detaching the completed device from the transport substrate. It is required for such a flexible device obtained by being subjected to a high-temperature process to have heat resistance at a high temperature. In particular, in a case where a thin film transistor process in which low temperature polysilicon (LTPS) is used is performed, a process temperature may reach 500° C. Therefore, the polyimide film to be formed on the transport substrate is required not to be thermally decomposed by hydrolysis and to have high heat resistance even during the high-temperature process. In addition, it is required for the polyimide film to secure transparency after processing as well as storage stability.

Therefore, in order to manufacture a flexible device, there is a need for polyimide having high heat resistance and preventing hydrolysis, thereby implementing excellent chemical resistance and storage stability and improving optical and mechanical properties.

In such a polyimide field, an aromatic polyimide resin has attracted attention as a resin having excellent thermal dimensional stability. A polyimide film, which is a molded article formed of aromatic polyimide having a rigid and linear chemical structure, has been widely used in a field requiring high thermal dimensional stability (a low coefficient of linear thermal expansion), such as a base film of a flexible substrate or an interlayer insulating film of a semiconductor. However, since the aromatic polyimide resin exhibits poor processability and brown coloration due to an intramolecular interaction and charge transfer complex (CTC), it is difficult to apply the aromatic polyimide resin to an optical use. In addition, since the polyimide has a significantly strong intermolecular force, the polyimide is insufficient in processability. In order to overcome such problems, attempts have been made to introduce an aliphatic chain, a flexible bonding group, a fluorinated functional group, and the like into a monomer for preparing polyimide. However, there is a problem in that mechanical properties, which are advantages of the polyimide, are deteriorated due to the introduction of these substituents.

These physical properties of the polyimide are derived from a monomer used for preparing the same. Thus, it is required to develop a monomer in order to prepare polyimide having further improved physical properties.

### RELATED ART DOCUMENT

### Patent Document

WO 2017-111299 A1 (Jul. 4, 2017)

### SUMMARY OF THE INVENTION

An embodiment of the present invention is directed to providing a diazaspiro compound having a structure and a use thereof.

Another embodiment of the present invention is directed to providing a diazaspiro compound having a structure that is significantly useful as a monomer by which a highly transparent polyimide film having excellent optical properties and improved Young's modulus properties may be produced.

Still another embodiment of the present invention is directed to providing a polyimide-based polymer composition containing the diazaspiro compound.

Still another embodiment of the present invention is directed to providing a polyimide precursor for producing a colorless and transparent polyimide film having flexibility using the diazaspiro compound.

Still another embodiment of the present invention is directed to providing a colorless and transparent polyimide film having flexibility using the polyimide precursor.

Still another embodiment of the present invention is directed to providing a laminate and a photoelectric device that include the polyimide film.

In one general aspect, there is provided a diazaspiro compound having a structure, and the diazaspiro compound is represented by the following Chemical Formula 1:

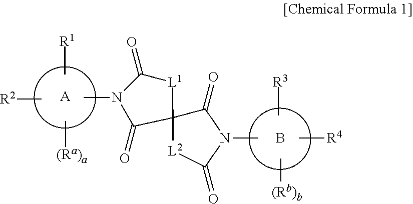

in Chemical Formula 1,

a ring A and a ring B are each independently a C6-C20 aromatic ring or a C3-C20 heteroaromatic ring;

L¹ and L² are each independently substituted or unsubstituted hydrocarbylene;

R¹ and R³ are each independently halogen, substituted or unsubstituted hydrocarbyl, —OR^{a11}, —NR^{a12}R^{a13}, —COR^{a14}, —COOR^{a15}, nitro, —SiR^{a16}R^{a17}R^{a18}, or cyano;

R² and R⁴ are each independently hydrogen, halogen, substituted or unsubstituted hydrocarbyl, —OR^{b11}, —NR^{b12}R^{b13}, —COR^{b14}, —COOR^{b15}, nitro, —SiR^{b16}R^{b17}R^{b18}, or cyano;

R¹ and R² may be linked to each other to form a fused ring, and R³ and R⁴ may be linked to each other to form a fused ring;

R^{a} and R^{b} are each independently halogen, substituted or unsubstituted hydrocarbyl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring;

R^{a11} to R^{a18}, R^{b11} to R^{b18}, and R^{c11} to R^{c18} are each independently hydrogen or substituted or unsubstituted hydrocarbyl; and

a and b are each independently an integer of 0 to 2, when a is an integer of 2, each of the R^{a}(s) may be the same as or different from each other, and when b is an integer of 2, each of the R^{b}(s) may be the same as or different from each other.

In an exemplary embodiment, the substituted hydrocarbylene and the substituted hydrocarbyl may be hydrocarbylene and hydrocarbyl substituted with one or more selected from the group consisting of halogen, hydroxy, C1-C10 alkyl, halo C1-C10 alkyl, C1-C10 alkoxy, —NR′R″ (where R′ and R″ are each independently hydrogen, C1-C10 alkyl, or C6-C20 aryl), nitro, and cyano.

In an exemplary embodiment, the diazaspiro compound may be represented by the following Chemical Formula 2:

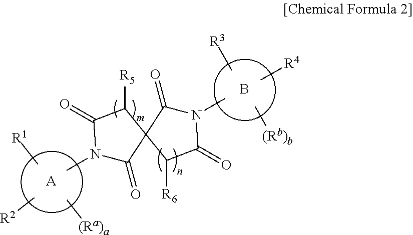

in Chemical Formula 2, a ring A, a ring B, R¹, R², R³, R⁴, R^{a}, R^{b}, a, and b are the same as defined in Chemical Formula 1;

R⁵ and R⁶ are each independently hydrogen, halogen, C1-C10 alkyl, or halo C1-C10 alkyl; and

m and n are each independently an integer of 1 to 5.

In an exemplary embodiment, in Chemical Formula 2, R¹ and R³ are each independently halogen, C1-C10 alkyl, halo C1-C10 alkyl, C3-C10 cycloalkyl, C6-C20 aryl, —OR^{a11}, —NR^{a12}R^{a13}, —COOR^{a15}, nitro, —SiR^{a16}R^{a17}R^{a18}, or cyano; R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano; R¹ and R² may be linked to each other by

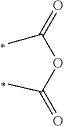

to form a fused ring, and R³ and R⁴ may be linked to each other by

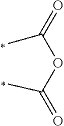

to form a fused ring; R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring; R^{a11}, R^{a12}, R^{a13}, R^{a15}, R^{a16}, R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl; R^{a17}, R^{a18}, R^{b17}, R^{b18}, R^{c17}, and R^{c18} are each independently C1-C10 alkyl, C3-C10 cycloalkyl, or C6-C20 aryl; and a and b may be each independently an integer of 0 or 1.

In an exemplary embodiment, the diazaspiro compound may be represented by the following Chemical Formula 3:

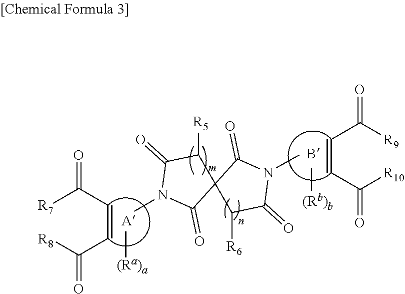

in Chemical Formula 3

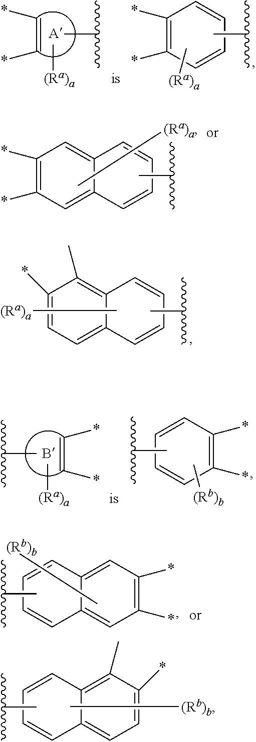

where a wavy line [symbol] represents a bonding site to a nitrogen atom, and an asterisk (*) represents a bonding site to a carbon atom;

R⁵ and R⁶ are each independently hydrogen, halogen, C1-C10 alkyl, or halo C1-C10 alkyl;

R⁷ to R¹⁰ are each independently hydroxy, C1-C10 alkoxy, C3-C10 cycloalkyloxy, or C6-C20 aryloxy, and R⁷ and R⁸, and R⁹ and R¹⁰ may be linked to each other by *—O—* to form a fused ring;

R^{a} and R^{b} are each independently halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

a and b are each independently an integer of 0 to 2; and

m and n are each independently an integer of 1 to 3.

In an exemplary embodiment, the diazaspiro compound may be represented by the following Chemical Formula 3-1, 3-2, or 3-3:

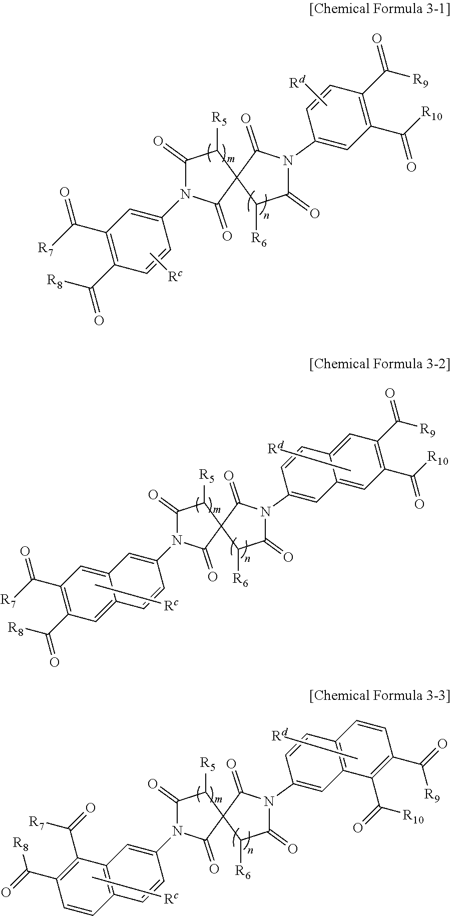

in Chemical Formula 3-1, 3-2, or 3-3,

R^{c} and R^{d} are each independently hydrogen, halogen, C1-C6 alkyl, halo C1-C6 alkyl, hydroxy, C1-C6 alkoxy, —SiR^{e1}R^{e2}R^{e3}, or cyano;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl;

R⁷ to R¹⁰ are each independently hydroxy or C1-C6 alkoxy, and R⁷ and R⁸, and R⁹ and R¹⁰ may be linked to each other by *—O—* to form a fused ring; and

m and n are each independently an integer of 1 or 2.

In an exemplary embodiment, the diazaspiro compound of Chemical Formula 3 may be selected from the following compounds, but is not limited thereto:

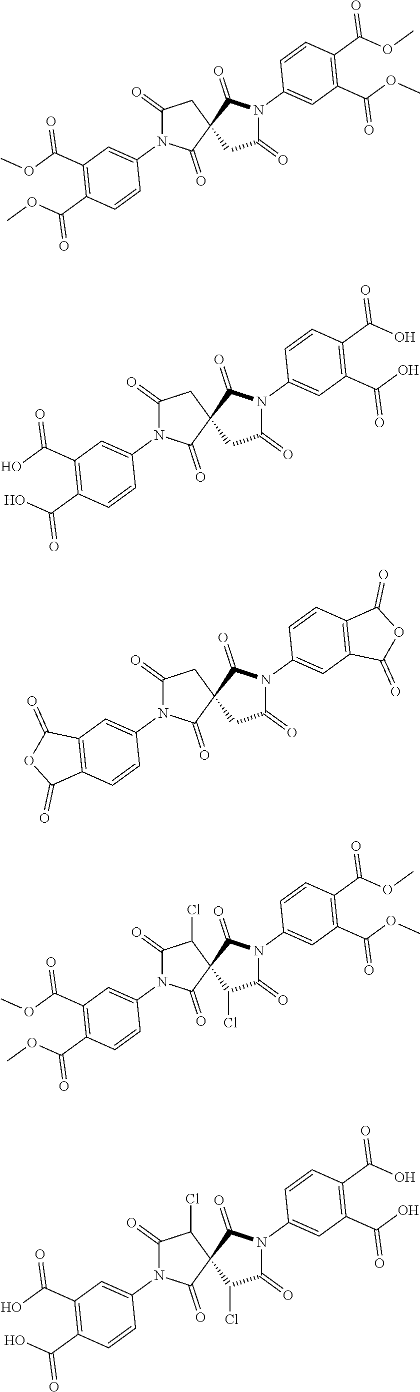

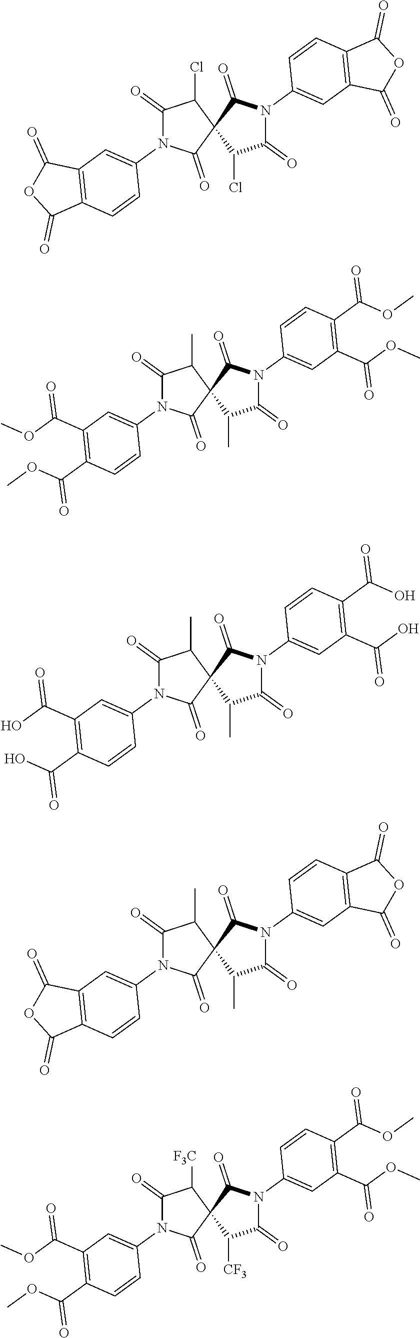

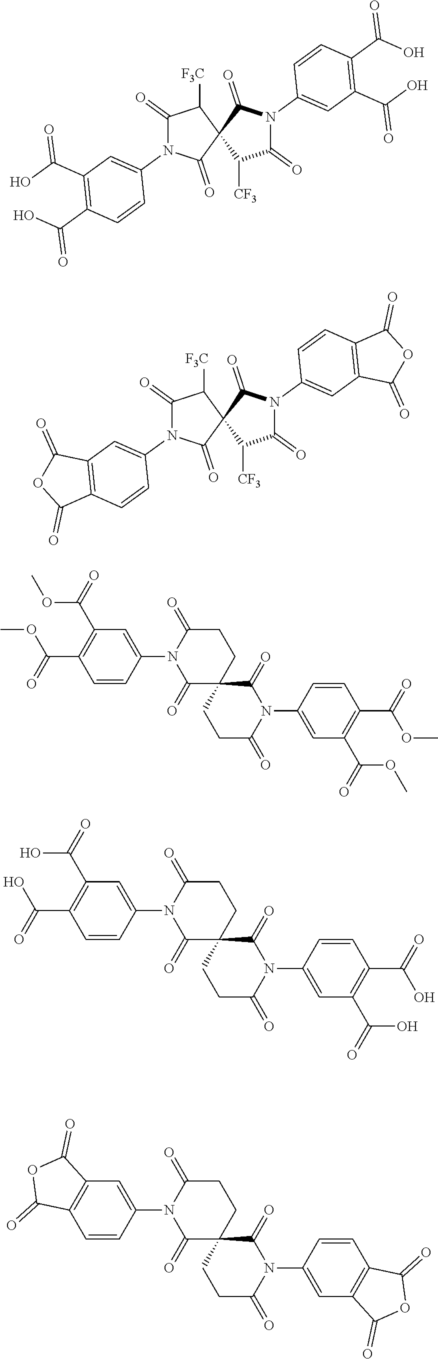

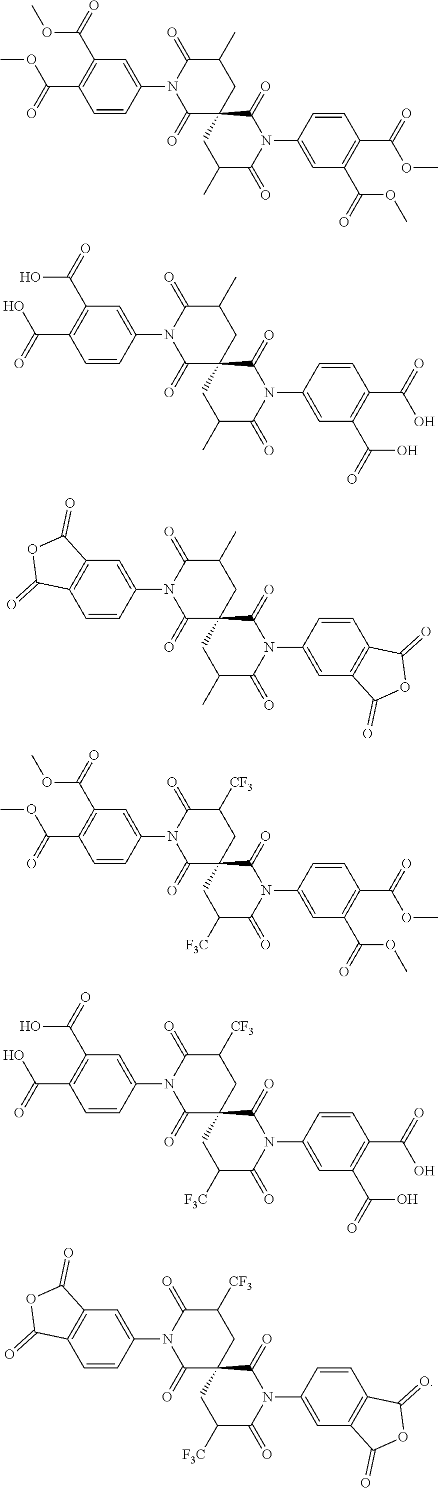

In an exemplary embodiment, the diazaspiro compound may be represented by the following Chemical Formula 4:

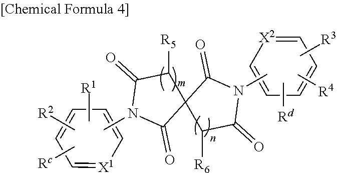

in Chemical Formula 4,

each of X¹ and X² is CR or N;

R¹ and R³ are each independently —NH₂ or nitro;

R², R⁴, R, R^{c}, and R^{d} are each independently hydrogen, halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano, and R² and R^{c}, and R⁴ and R^{d} may be linked to each other by C3-C5 alkylene or C3-C5 alkenylene to form a fused ring;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl; and

m and n are each independently an integer of 1 to 3.

In an exemplary embodiment, the diazaspiro compound may be represented by the following Chemical Formula 4-1:

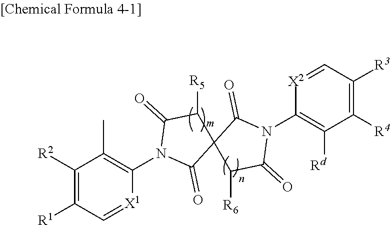

in Chemical Formula 4-1,

each of X¹ and X² is CR or N;

R¹ and R³ are each independently —NH₂ or nitro;

R², R⁴, R, Re, and R^{d} are each independently hydrogen, halogen, C1-C6 alkyl, halo C1-C6 alkyl, hydroxy, C1-C6 alkoxy, —SiR^{e1}R^{e2}R^{e3}, or cyano, and R² and R^{c}, and R⁴ and R^{d} may be linked to

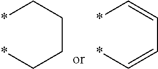

each other by or to form a fused ring;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl; and

m and n are each independently an integer of 1 or 2.

In an exemplary embodiment, the diazaspiro compound of Chemical Formula 4 may be selected from the following compounds, but is not limited thereto:

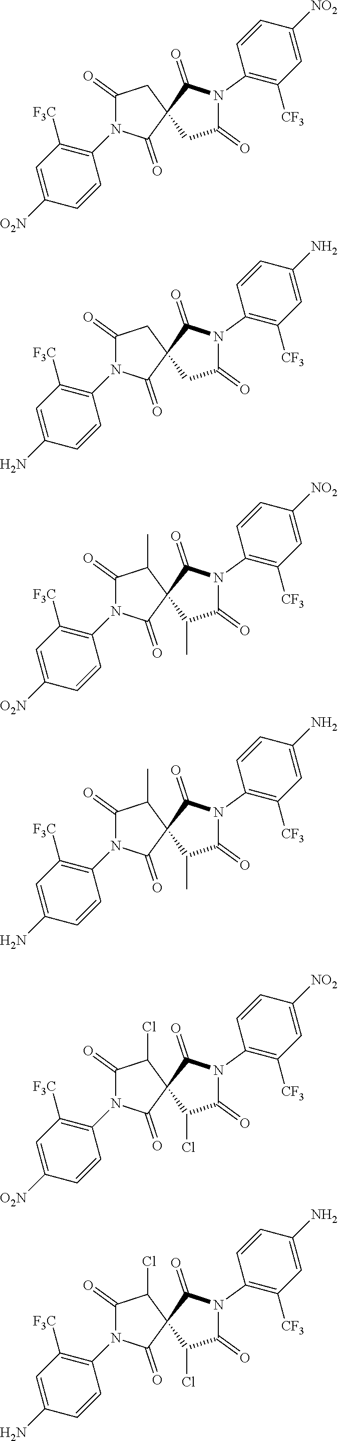

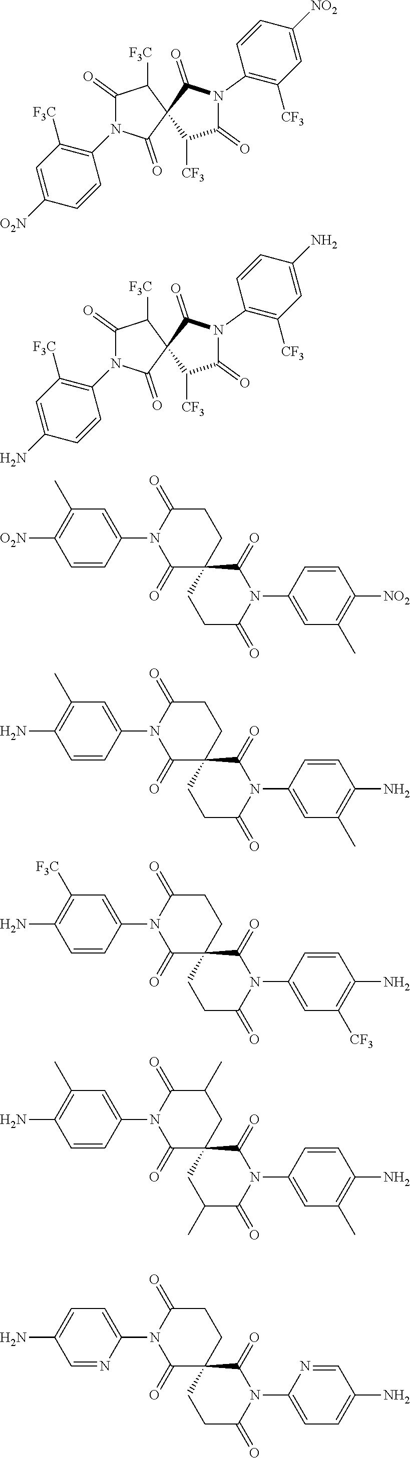

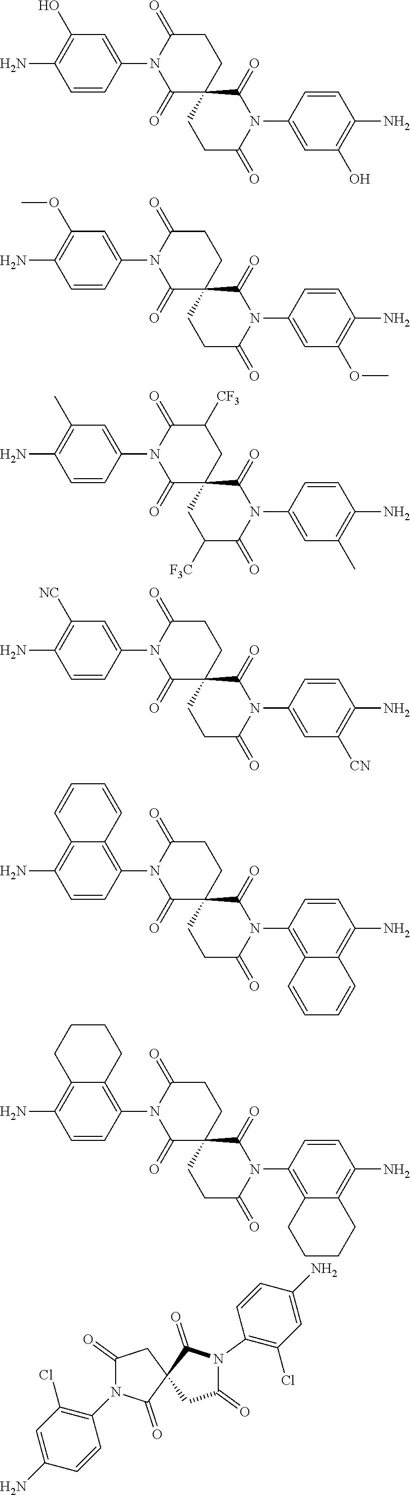

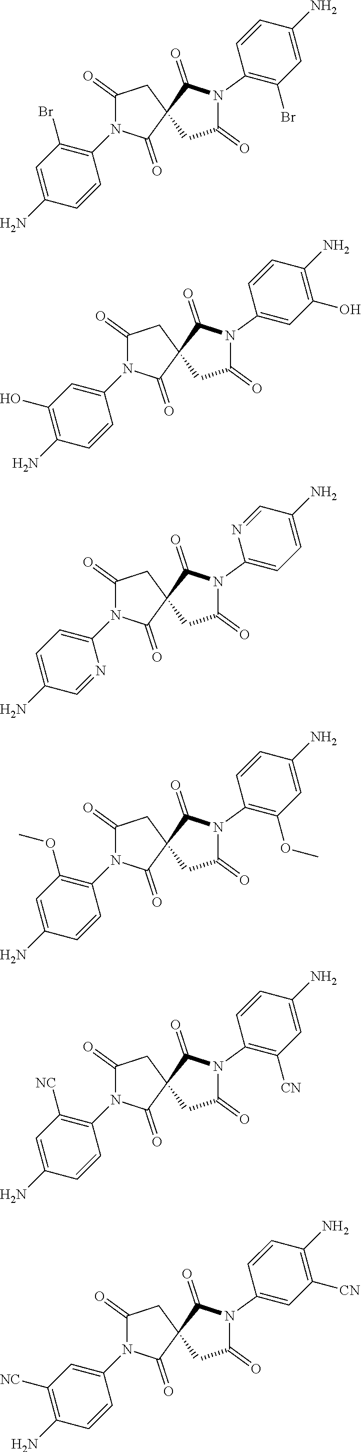

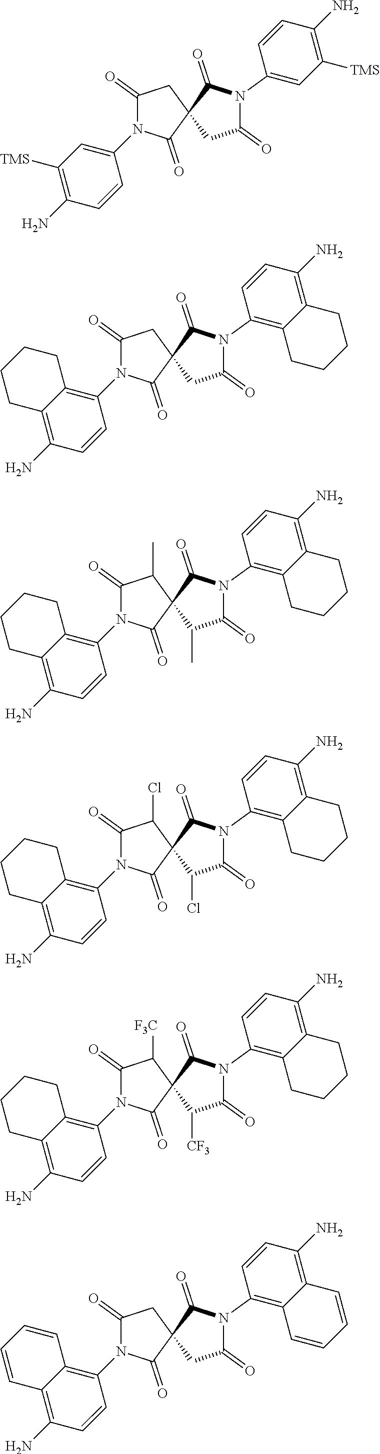

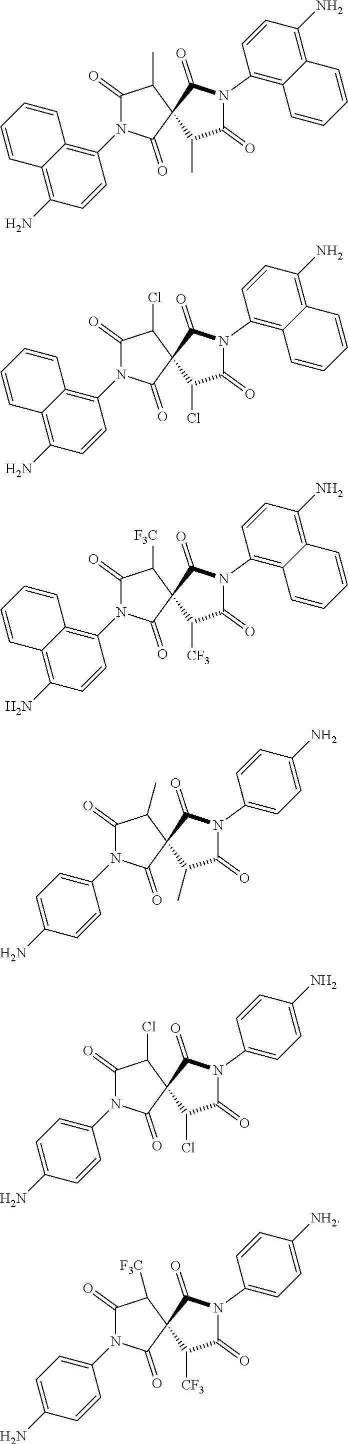

In another general aspect, there is provided a polyimide-based polymer composition containing a diazaspiro compound represented by the following Chemical Formula 1-1 or 1-2:

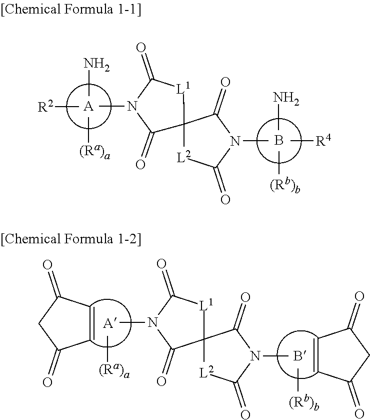

in Chemical Formulas 1-1 and 1-2,

a ring A, a ring B, a ring A′, and a ring B′ are each independently a C6-C20 aromatic ring or a C3-C20 heteroaromatic ring;

L¹ and L² are each independently substituted or unsubstituted hydrocarbylene;

R² and R⁴ are each independently hydrogen, halogen, substituted or unsubstituted hydrocarbyl, —OR^{b11}, —NR^{b12}R^{b13}, —COR^{b14}, —COOR^{b15}, nitro, —SiR^{b16}R^{b17}R^{b18}, or cyano;

R^{a} and R^{b} are each independently halogen, substituted or unsubstituted hydrocarbyl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring;

R^{b11} to R^{b18} and R^{c11} to R^{c18} are each independently hydrogen or substituted or unsubstituted hydrocarbyl; and

a and b are each independently an integer of 0 to 2, when a is an integer of 2, each of the R^{a}(s) may be the same as or different from each other, and when b is an integer of 2, each of the R^{b}(s) may be the same as or different from each other.

In an exemplary embodiment, the polyimide-based polymer may be polyimide, a polyimide precursor, or a mixture thereof.

In still another general aspect, there is provided a polyimide precursor for producing a polyimide film having improved physical properties, and the polyimide precursor of the present invention has a structural unit derived from a diazaspiro compound represented by Chemical Formula 1-1 or 1-2.

In an exemplary embodiment, the polyimide precursor may have a structural unit derived from the diazaspiro compound of Chemical Formula 1-1 and a structural unit derived from an acid dianhydride compound.

In an exemplary embodiment, the acid dianhydride compound may be represented by the following Chemical Formula D:

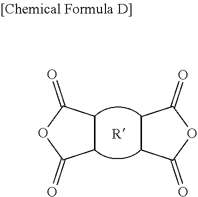

in Chemical Formula D,

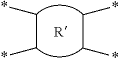

is at least one tetravalent group selected from

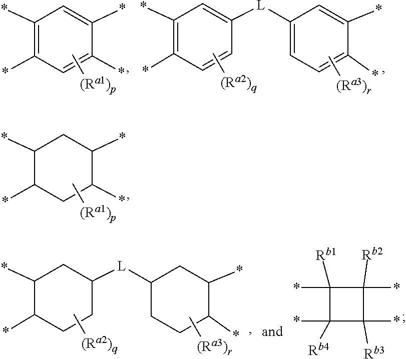

R^{a1}, R^{a2}, and R^{a3} are each independently C1-C10 alkyl or halo C1-C10 alkyl;

L is a single bond, C1-C10 alkylene, —O—, —S—, —CO—, —SO₂—, —SiR^{f1}R^{f2}—, —CO—Ar—CO—, or —O—Ar¹—(X—Ar²)ₛ—O—, and the alkylene may be further substituted with one or more selected from C1-C10 alkyl and halo C1-C10 alkyl;

R^{f1} and R^{f2} are each independently C1-C10 alkyl;

Ar, Ar¹, and Ar² are each independently C6-C20 arylene, and the arylene may be further substituted with one or more selected from C1-C10 alkyl and halo C1-C10 alkyl;

X is O or S;

R^{b1} to R^{b4} are each independently hydrogen, C1-C10 alkyl, or C6-C20 aryl;

p, q, and r are each independently an integer of 0 to 2; and

s is an integer of 0 or 1.

In an exemplary embodiment, the polyimide precursor may have a repeating unit represented by the following Chemical Formula 11:

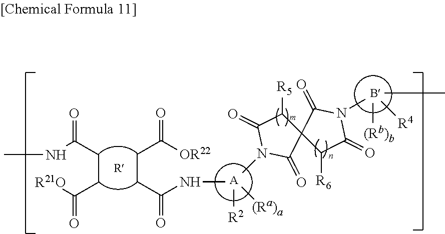

in Chemical Formula 11,

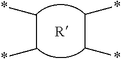

is the same as defined in Chemical Formula D;

R²¹ and R²² are each independently hydrogen or C1-C10 alkyl;

a ring A and a ring B are each independently a C6-C20 aromatic ring or a C3-C20 heteroaromatic ring;

R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;

R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;

R^{b17}, R^{b18}, R^{c17}, and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1; and

m and n are each independently an integer of 1 to 5.

In an exemplary embodiment, the polyimide precursor may have a structural unit derived from the diazaspiro compound of Chemical Formula 1-2 and a structural unit derived from a diamine compound.

In an exemplary embodiment, the diamine compound may be represented by the following Chemical Formula E:

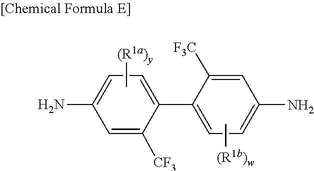

in Chemical Formula E,

R^{1a} and R^{1b} are each independently halogen, C1-C10 alkyl, halo C1-C10 alkyl, C1-C10 alkoxy, or C6-C12 aryl; and

y and w are each independently an integer of 0 to 3.

In an exemplary embodiment, the polyimide precursor may have a repeating unit represented by the following Chemical Formula 12:

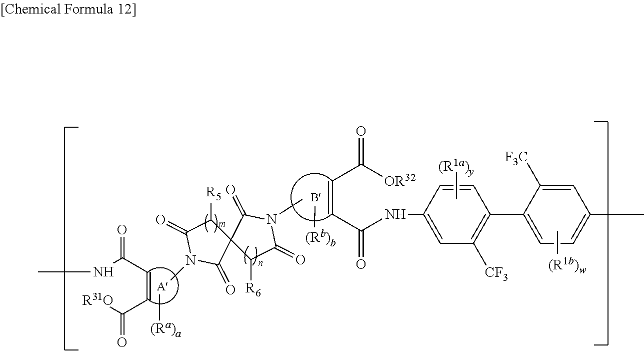

in Chemical Formula 12, R^{1a}, R^{1b}, y, and w are the same as defined in Chemical Formula E;

R³¹ and R³² are each independently hydrogen or C1-C10 alkyl;

a ring A′ and a ring B′ are each independently a C6-C20 aromatic ring or a C3-C20 heteroaromatic ring;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;

R^{c11}, R^{c12}, R^{c13}, R^{c15}, and R^{c16} are each independently hydrogen, C1-C10 alkyl, C3-C10 cycloalkyl, or C6-C20 aryl;

R^{c17} and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1; and

m and n are each independently an integer of 1 to 5.

In still another general aspect, there is provided a polyimide film produced using the polyimide precursor.

In an exemplary embodiment, the polyimide film may have a repeating unit represented by the following Chemical Formula 13:

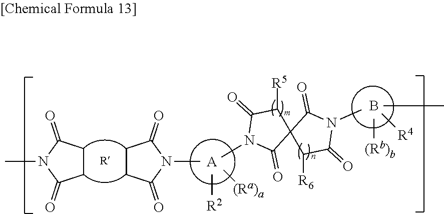

in Chemical Formula 13,

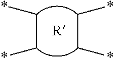

is at least one tetravalent group selected from

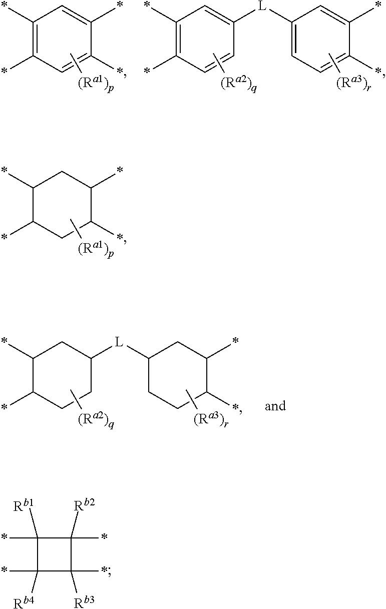

R^{a1}, R^{a2}, and R^{a3} are each independently C1-C10 alkyl or halo C1-C10 alkyl;

L is a single bond, C1-C10 alkylene, —O—, —S—, —CO—, —SO₂—, —SiR^{f1}R^{f2}—, —CO—Ar—CO—, or —O—Ar¹—(X—Ar²)ₛ—O—, and the alkylene may be further substituted with one or more selected from C1-C10 alkyl and halo C1-C10 alkyl;

R^{f1} and R^{f2} are each independently C1-C10 alkyl;

Ar, Ar¹, and Ar² are each independently C6-C20 arylene, and the arylene may be further substituted with one or more selected from C1-C10 alkyl and halo C1-C10 alkyl;

X is O or S;

R^{b1} to R^{b4} are each independently hydrogen, C1-C10 alkyl, or C6-C20 aryl;

a ring A and a ring B are each independently a C6-C20 aromatic ring or a C3-C20 heteroaromatic ring;

R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;

R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;

R^{b17}, R^{b18}, R^{c17}, and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1;

m and n are each independently an integer of 1 to 5;

p, q, and r are each independently an integer of 0 to 2; and

s is an integer of 0 or 1.

In an exemplary embodiment, the polyimide film may have a repeating unit represented by the following Chemical Formula 14:

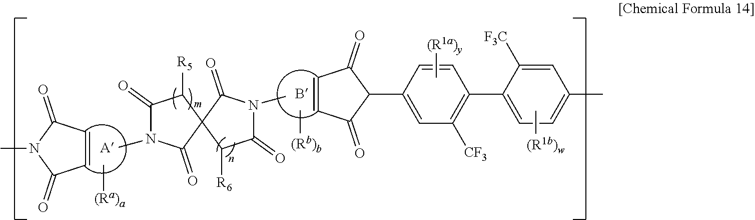

in Chemical Formula 14,

a ring A′ and a ring B′ are each independently a C6-C20 aromatic ring or a C3-C20 heteroaromatic ring;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;

R^{c11}, R^{c12}, R^{c13}, R^{c15}, and R^{c16} are each independently hydrogen, C1-C10 alkyl, C3-C10 cycloalkyl, or C6-C20 aryl;

R^{c17} and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

R^{1a} and R^{1b} are each independently halogen, C1-C10 alkyl, halo C1-C10 alkyl, C1-C10 alkoxy, or C6-C12 aryl;

a and b are each independently an integer of 0 or 1;

m and n are each independently an integer of 1 to 5; and

y and w are each independently an integer of 0 to 3.

In an exemplary embodiment, the polyimide film may have a yellow index (YI) of 2.0 or less, a light transmittance of 90% or more, a haze of 0.5% or less, and a Young's modulus of 4.5 GPa or less.

In an exemplary embodiment, the polyimide film may be used for a device substrate, a display device substrate, an optical film, an integrated circuit (IC) package, an adhesive film, a multi-layer flexible printed circuit (FPC), a tape, a touch panel, or an optical disk protective film.

Other features and aspects will be apparent from the following detailed description, the drawings, and the claims.

### DESCRIPTION OF THE INVENTION

Unless otherwise defined, all the technical terms and scientific terms used in the present invention have the same meanings as commonly understood by those skilled in the art to which the present invention pertains. The terms used in the description of the present invention are merely used to effectively describe a specific exemplary embodiment, but are not intended to limit the present invention.

Unless the context clearly indicates otherwise, the singular forms used in the specification may be intended to include the plural forms.

In addition, units used in the present specification without special mention are based on weight, and as an example, a unit of % or a ratio refers to wt % or a weight ratio. Unless otherwise defined, wt % refers to wt % of any one component in a composition with respect to a total weight of the composition.

In addition, a numerical range used in the present specification includes upper and lower limits and all values within these limits, increments logically derived from a form and span of a defined range, all double limited values, and all possible combinations of the upper and lower limits in the numerical range defined in different forms. Unless otherwise particularly defined in the present specification, all values out of the numerical range that may occur due to the rounding off of the experimental errors or values also fall within the defined numerical ranges.

In the present specification, the expression “comprise(s)” is intended to be an open-ended transitional phrase having an equivalent meaning to “include(s)”, “contain(s)”, “have (has)”, and “are (is) characterized by”, and does not exclude elements, materials, or steps, all of which are not further recited herein.

The terms “polyimide precursor composition”, “polyimide precursor solution”, “polyimide-based polymer composition”, and “polyimide-based polymer solution” used in the present invention refer to compositions for preparing a polyimide-based polymer. Specifically, the polyimide precursor may have a meaning equivalent to a polyamic acid or a polyamic acid ester. In addition, the polyimide precursor solution may also be used as a composition for preparing polyamideimide.

The term “polyimide film” used in the present specification refers to a film formed of a polymer containing an imide bond. Examples of the polymer include all polymers containing both an imide bond and another bond, and the polymer may be, for example, polyamideimide containing an amide bond.

The term “polyimide-based polymer” used in the present specification refers to both polyimide and a polyimide precursor (that is, a polyamic acid and a polyamic acid ester).

The term “aromatic ring” or “heteroaromatic ring” used in the present specification is a ring in which electrons are delocalized or resonated, and the “hetero” means that carbon of the aromatic ring is substituted with one or more heteroatoms selected from N, O, S, and P.

The terms “substituent”, “radical”, “group”, “moiety”, and “fragment” used in the present specification may be used interchangeably.

The term “C_{A}-C_{B}” used in the present specification means that “the number of carbon atoms is A or more and B or less”, and the term “A to B” means that “A or more and B or less”.

The term “aromatic” used in the present specification satisfies the Huckel's Rule, and according to the Huckel's Rule, refers to a case in which i) 4n+2 electrons that are completely conjugated by empty p-orbitals, unsaturated bonds, unpaired electron pairs, and the like exist; ii) 4n+2 electrons should form a planar isomer and a ring structure; and iii) all atoms of the ring are involved in conjugation.

The term “hydrocarbon” used in the present specification refers to any one of a linear, branched, or cyclic alkane, alkene, or alkyne, and an aromatic hydrocarbon.

The term “hydrocarbyl” used in the present specification refers to a radical having one bonding site derived from a hydrocarbon, and includes alkyl, alkenyl, alkynyl, cycloalkyl, cycloalkenyl, aryl, or a combination thereof.

The term “hydrocarbylene” used in the present specification refers to a radical having two bonding sites derived from a hydrocarbon, and includes alkylene, alkenylene, alkynylene, cycloalkylene, cycloalkenylene, arylene, or a combination thereof.

The term “substitution” used in the present specification means that a hydrogen atom bonded to a carbon atom is substituted with another substituent, and a position to be substituted is not particularly limited as long as it is a position at which the hydrogen atom is substituted, that is, a position at which the substituent may be substituted. When two or more substituents are substituted, the two or more substituents may be the same as or different from each other.

In the description of “substituted or unsubstituted” in the present specification, the phrase “substituted” means that at least one hydrogen atom in a certain functional group is replaced by another atom or another functional group (that is, a substituent). The description of “substituted hydrocarbylene and the substituted hydrocarbyl” means that the hydrocarbylene and the hydrocarbyl are each independently substituted with one or more substituents selected from the group consisting of halogen, hydroxy, C1-C10 alkyl, halo C1-C10 alkyl, C1-C10 alkoxy, —NR′R″ (where R′ and R″ are each independently hydrogen, C1-C10 alkyl, or C6-C20 aryl), nitro, and cyano.

The term “halo” or “halogen” used in the present specification refers to a fluorine (F), chloride (Cl), bromine (Br), or iodine (I) atom.

The term “alkyl” used in the present specification refers to an organic radical derived from an aliphatic saturated hydrocarbon by removing one hydrogen atom and includes both linear and branched forms.

The term “alkoxy” used in the present specification is represented by “*—O-alkyl”, and the alkyl is the same as defined above.

The term “haloalkyl” used in the present specification refers to the alkyl in which one hydrogen atom is substituted with a halogen atom.

The term “cycloalkyl” used in the present specification refers to a monovalent saturated carbocyclic radical consisting of one or more rings.

The term “aryl” used in the present specification refers to an organic radical derived from an aromatic hydrocarbon by removing one hydrogen atom, includes a monocyclic or fused ring system having suitably 4 to 7 ring atoms, and preferably 5 or 6 ring atoms in each ring, and even includes a form in which a plurality of aryls are linked by a single bond. Examples of the aryl include, but are not limited to, phenyl, naphthyl, biphenyl, and terphenyl.

The present invention provides a diazaspiro compound having a structure, and the diazaspiro compound having a structure according to the present invention is represented by the following Chemical Formula 1:

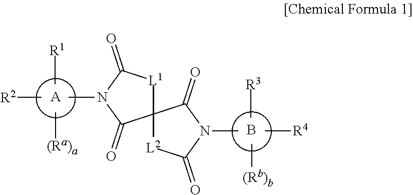

in Chemical Formula 1,

a ring A and a ring B are each independently a C6-C20 aromatic ring or a C3-C20 heteroaromatic ring;

L¹ and L² are each independently substituted or unsubstituted hydrocarbylene;

R¹ and R³ are each independently halogen, substituted or unsubstituted hydrocarbyl, —OR^{a11}, —NR^{a12}R^{a13}, —COR^{a14}, —COOR^{a15}, nitro, —SiR^{a16}R^{a17}R^{a18}, or cyano;

R² and R⁴ are each independently hydrogen, halogen, substituted or unsubstituted hydrocarbyl, —OR^{b11}, —NR^{b12}R^{b13}, —COR^{b14}, —COOR^{b15}, nitro, —SiR^{b16}R^{b17}R^{b18}, or cyano;

R¹ and R² may be linked to each other to form a fused ring, and R³ and R⁴ may be linked to each other to form a fused ring;

R^{a} and R^{b} are each independently halogen, substituted or unsubstituted hydrocarbyl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring;

R^{a11} to R^{a18}, R^{b11} to R^{b18}, and R^{c11} to R^{c18} are each independently hydrogen or substituted or unsubstituted hydrocarbyl; and

a and b are each independently an integer of 0 to 2, when a is an integer of 2, each of the R^{a}(s) may be the same as or different from each other, and when b is an integer of 2, each of the R^{b}(s) may be the same as or different from each other.

The diazaspiro compound having a structure according to the present invention is a spiro compound of an azacyclic dione containing one nitrogen atom and carbon atoms of two carbonyl groups as ring atoms, in which an aromatic ring or a heteroaromatic ring is substituted for the nitrogen atom, and the aromatic ring or the heteroaromatic ring is substituted with a specific substituent.

In particular, in a case where an amino group (—NH₂) is substituted for the aromatic ring or the heteroaromatic ring, the diazaspiro compound may be significantly useful as a diamine monomer for synthesizing a polyimide-based polymer, and in a case where an acid anhydride group (—C(═O)—O—C(═O)—) is fused at the aromatic ring or the heteroaromatic ring, the diazaspiro compound may be significantly useful as an acid dianhydride monomer for synthesizing a polyimide-based polymer.

As such, a polyimide film having improved physical properties may be produced by using the diazaspiro compound according to the present invention as a diamine monomer or an acid dianhydride monomer.

In a case where the diazaspiro compound according to the present invention is used as a diamine monomer or an acid dianhydride monomer, strong steric hindrance and charge transfer complex (CT-complex) caused in the polyimide main chain may be significantly suppressed by the structural characteristics described above, and inhibition of formation of a resonance structure and a pi electron density may be efficiently reduced, such that a colorless, transparent, and flexible polyimide film having excellent optical properties may be produced.

According to an exemplary embodiment of the present invention, in Chemical Formula 1, L¹ and L² may be each independently substituted or unsubstituted C1-C10 alkylene.

According to an exemplary embodiment of the present invention, Chemical Formula 1 may be represented by the following Chemical Formula 2:

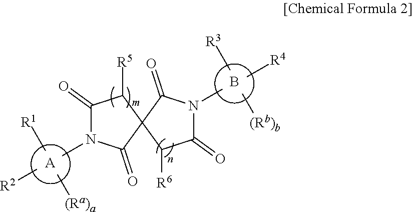

in Chemical Formula 2, a ring A, a ring B, R¹, R², R³, R⁴, R^{a}, R^{b}, a, and b are the same as defined in Chemical Formula 1;

R⁵ and R⁶ are each independently hydrogen, halogen, C1-C10 alkyl, or halo C1-C10 alkyl; and

m and n are each independently an integer of 1 to 5.

In an exemplary embodiment of the present invention, R¹ and R³ are each independently halogen, substituted or unsubstituted C1-C10 alkyl, substituted or unsubstituted C3-C10 cycloalkyl, substituted or unsubstituted C6-C20 aryl, —OR^{a11}, —NR^{a12}R^{a13}, —COR^{a14}, —COOR^{a15}, nitro, —SiR^{a16}R^{a17}R^{a18}, or cyano; R² and R⁴ are each independently hydrogen, halogen, substituted or unsubstituted C1-C10 alkyl, substituted or unsubstituted C3-C10 cycloalkyl, substituted or unsubstituted C6-C20 aryl, —OR^{b11}, —NR^{b12}R^{b13}, —COR^{b14}, —COOR^{b15}, nitro, —SiR^{b16}R^{b17}R^{b18}, or cyano; R¹ and R² may be linked to each other to form a fused ring, and R³ and R⁴ may be linked to each other to form a fused ring; R^{a} and R^{b} are each independently halogen, substituted or unsubstituted C1-C10 alkyl, substituted or unsubstituted C3-C10 cycloalkyl, substituted or unsubstituted C6-C20 aryl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring; and R^{a11} to R^{a18}, R^{b11} to R^{b18}, and R^{c11} to R^{c18} may be each independently hydrogen, substituted or unsubstituted C1-C10 alkyl, substituted or unsubstituted C3-C10 cycloalkyl, or substituted or unsubstituted C6-C20 aryl.

In an exemplary embodiment of the present invention, R¹ and R³ are each independently halogen, C1-C10 alkyl, halo C1-C10 alkyl, C3-C10 cycloalkyl, C6-C20 aryl, —OR^{a11}, —NR^{a12}R^{a13}, —COOR^{a15}, nitro, —SiR^{a16}R^{a17}R^{a18}, or cyano; R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano; R¹ and R² may be linked to each other by

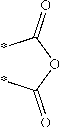 [SMILES: *C(=O)OC(*)=O]

to form a fused ring, and R³ and R⁴ may be linked to each other by

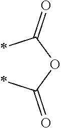 [SMILES: *C(=O)OC(*)=O]

to form a fused ring; R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring; R^{a11}, R^{a12}, R^{a13}, R^{a15}, R^{a16}, R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl; R^{a17}, R^{a18}, R^{b17}, R^{b18}, R^{c17}, and R^{c18} are each independently C1-C10 alkyl, C3-C10 cycloalkyl, or C6-C20 aryl; and a and b may be each independently an integer of 0 or 1.

In an exemplary embodiment of the present invention, the ring A and the ring B may be each independently a C6-C12 aromatic ring or a C3-C12 heteroaromatic ring.

In an exemplary embodiment of the present invention, the ring A and the ring B may be each independently benzene, naphthalene, anthracene, phenanthrene, pyrene, pyridine, pyridazine, pyrimidine, pyrazine, or triazine, and may be preferably benzene, naphthalene, or pyridine.

According to an exemplary embodiment of the present invention, Chemical Formula 2 may be represented by the following Chemical Formula 3:

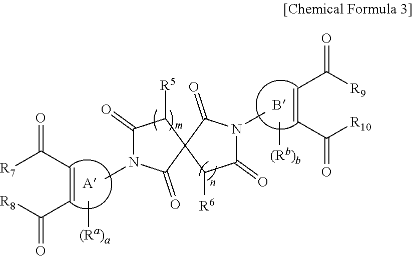

in Chemical Formula 3,

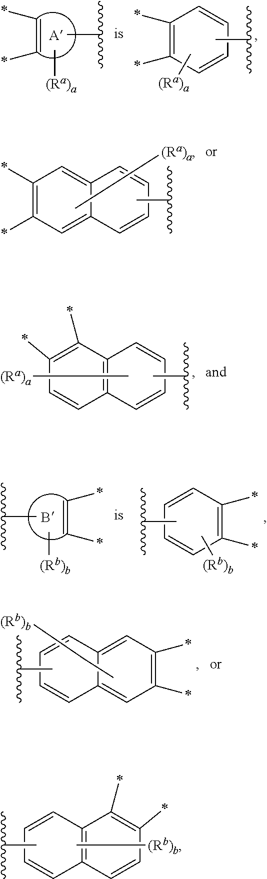

where wavy line [symbol] represents a bonding site to a nitrogen atom, and an asterisk (*) represents a bonding site to a carbon atom;

R⁵ and R⁶ are each independently hydrogen, halogen, C1-C10 alkyl, or halo C1-C10 alkyl;

R⁷ to R¹⁰ are each independently hydroxy, C1-C10 alkoxy, C3-C10 cycloalkyloxy, or C6-C20 aryloxy, and R⁷ and R⁸, and R⁹ and R¹⁰ may be linked to each other by *—O—* to form a fused ring;

R^{a} and R^{b} are each independently halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano;

R^{e1} is hydrogen, C1-C10 alkyl, C3-C10 cycloalkyl, or C6-C20 aryl; R^{e2} and R^{e3} are each independently C1-C10 alkyl, C3-C10 cycloalkyl, or C6-C20 aryl;

a and b are each independently an integer of 0 to 2; and

m and n are each independently an integer of 1 to 3.

In an exemplary embodiment of the present invention, all of R⁷ to R¹⁰ may be hydroxy.

In an exemplary embodiment of the present invention, all of R⁷ to R¹⁰ may be C1-C3 alkoxy.

In an exemplary embodiment of the present invention, R⁷ and R⁸, and R⁹ and R¹⁰ may be linked to each other by *—O—* to form a fused ring.

In an exemplary embodiment of the present invention, in a case where R⁷ and R⁸, and R⁹ and R¹⁰ are linked to each other by *—O—* to form a fused ring, the diazaspiro compound is useful as an acid dianhydride monomer for preparing a polyimide-based polymer having excellent optical properties and flexibility.

More preferably, according to an exemplary embodiment of the present invention, Chemical Formula 3 may be represented by the following Chemical Formula 3-1, 3-2, or 3-3:

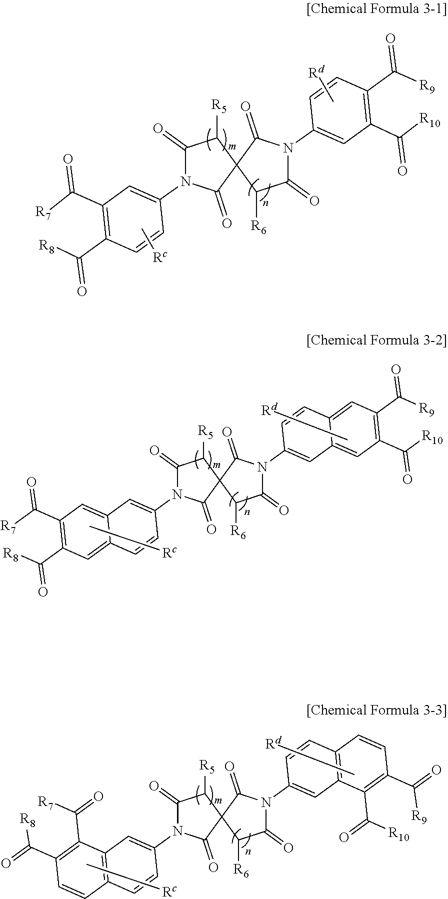

in Chemical Formula 3-1, 3-2, or 3-3,

R^{c} and R^{d} are each independently hydrogen, halogen, C1-C6 alkyl, halo C1-C6 alkyl, hydroxy, C1-C6 alkoxy, —SiR^{e1}R^{e2}R^{e3}, or cyano;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl;

R⁷ to R¹⁰ are each independently hydroxy or C1-C6 alkoxy, and R⁷ and R⁸, and R⁹ and R¹⁰ may be linked to each other by *—O—* to form a fused ring; and

m and n are each independently an integer of 1 or 2.

According to an exemplary embodiment of the present invention, the diazaspiro compound of Chemical Formula 3 may have a symmetrical structure.

According to an exemplary embodiment of the present invention, the diazaspiro compound of Chemical Formula 3 may be selected from the following compounds, but is not limited thereto:

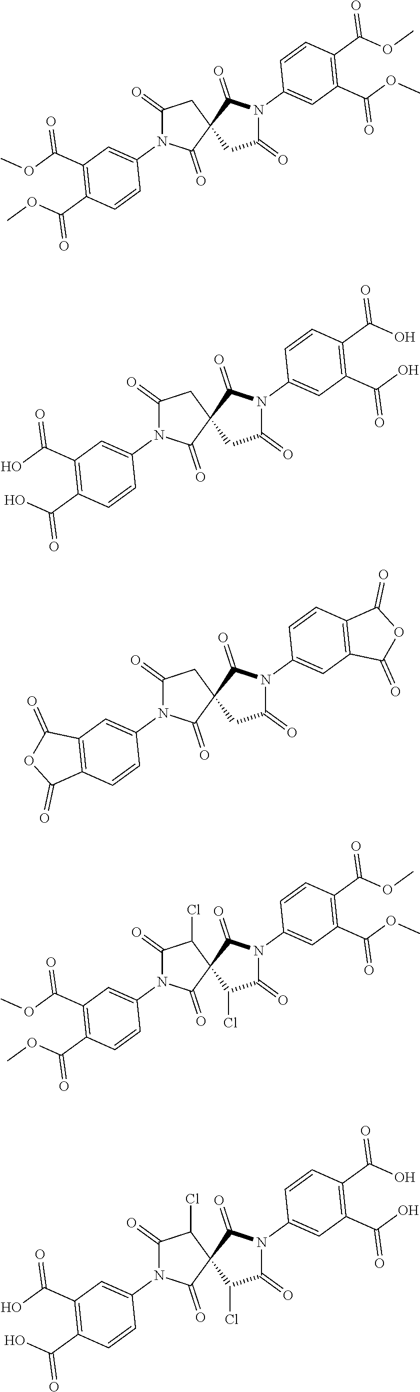

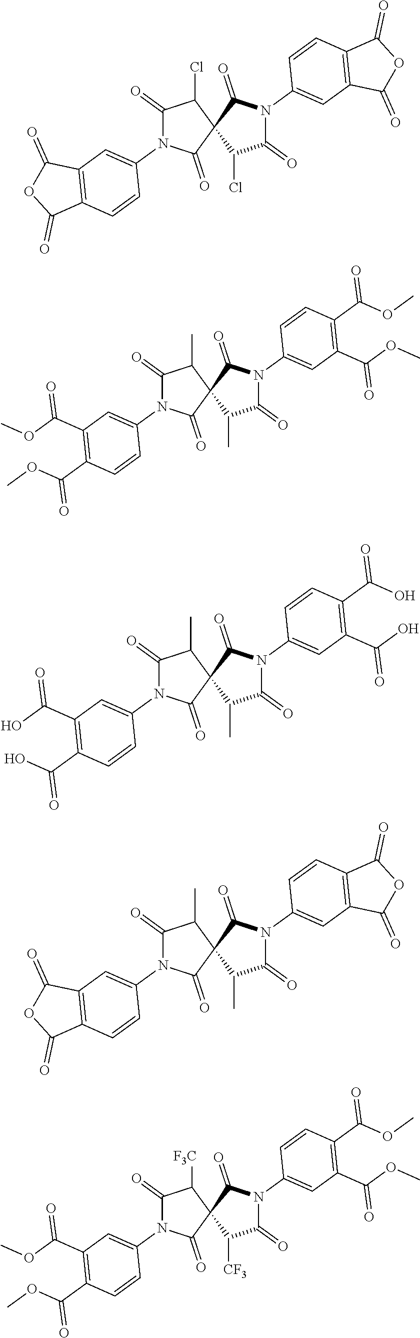

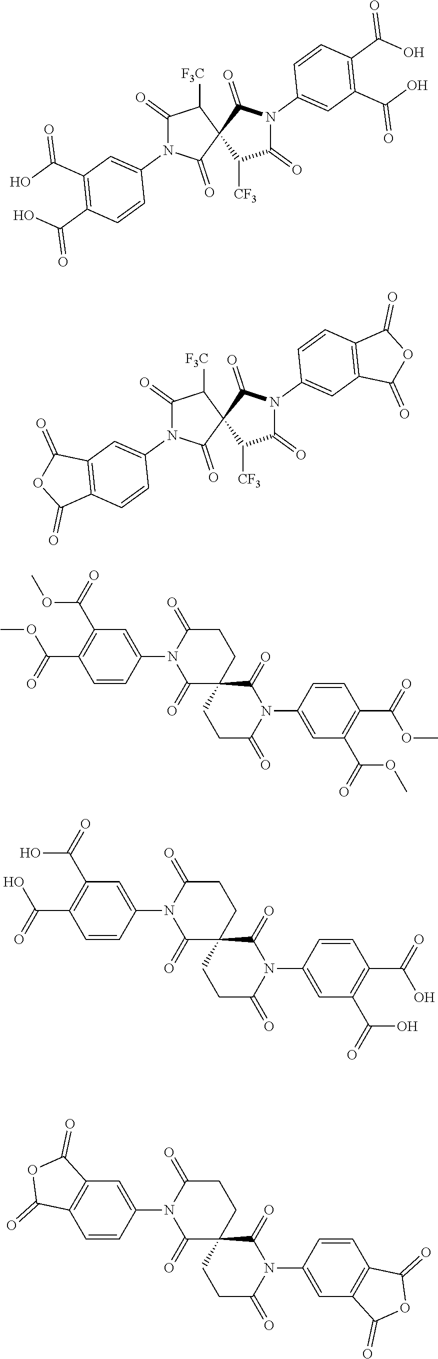

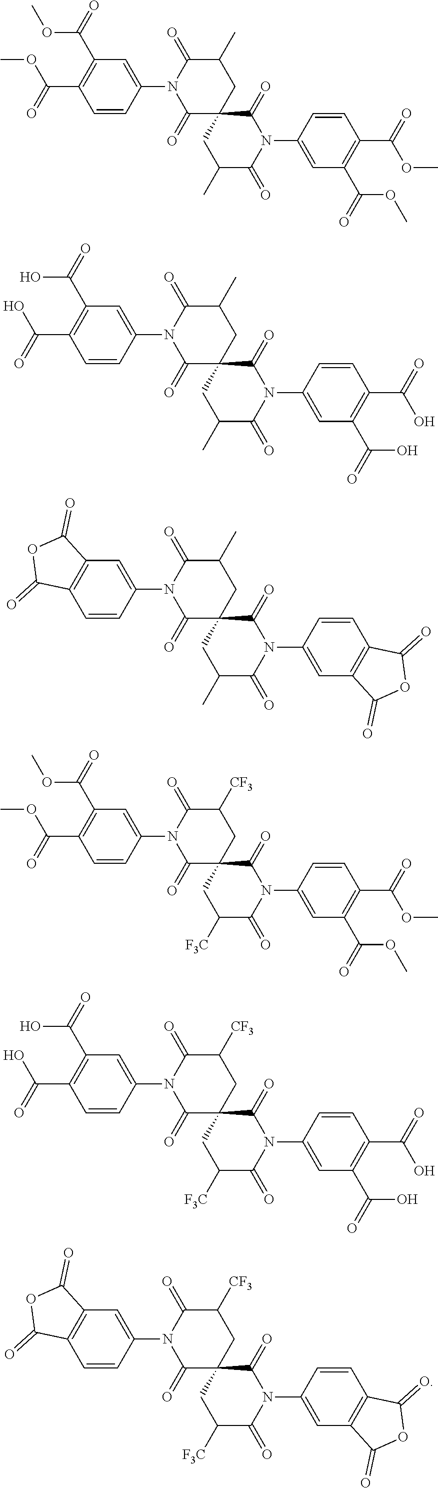

According to an exemplary embodiment of the present invention, Chemical Formula 2 may be represented by the following Chemical Formula 4:

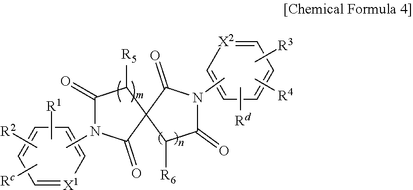

in Chemical Formula 4,

each of X¹ and X² is CR or N;

R¹ and R³ are each independently —NH₂ or nitro;

R², R⁴, R, R^{c}, and R^{d} are each independently hydrogen, halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano, and R² and R^{c}, and R⁴ and R^{d} may be linked to each other by C3-C5 alkylene or C3-C5 alkenylene to form a fused ring;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl; and

m and n are each independently an integer of 1 to 3.

As a specific example, the halo C1-C10 alkyl may be perhalo C1-C10 alkyl, and preferably, perfluoro C1-C10 alkyl.

In an exemplary embodiment of the present invention, all of R¹ and R³ may be nitro.

In an exemplary embodiment of the present invention, all of R¹ and R³ may be —NH₂.

According to an exemplary embodiment of the present invention, in Chemical Formula 4, in a case where all of R¹ and R³ are amino (—NH₂), the diazaspiro compound is significantly useful as a diamine monomer for preparing a highly transparent and flexible polyimide-based polymer having excellent optical properties and improved Young's modulus properties.

More preferably, according to an exemplary embodiment of the present invention, Chemical Formula 4 may be represented by the following Chemical Formula 4-1:

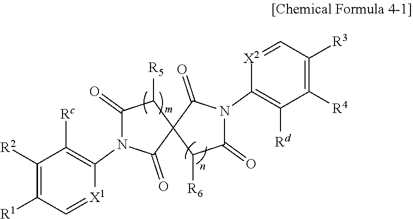

in Chemical Formula 4-1,

each of X¹ and X² is CR or N;

R¹ and R³ are each independently —NH₂ or nitro;

R², R⁴, R, R^{c}, and R^{d} are each independently hydrogen, halogen, C1-C6 alkyl, halo C1-C6 alkyl, hydroxy, C1-C6 alkoxy, —SiR^{e1}R^{e2}R^{e3}, or cyano, and R² and R^{c}, and R⁴ and R^{d} may be linked to each other by

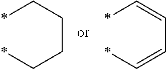

to form a fused ring;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl; and

m and n are each independently an integer of 1 or 2.

As a specific example, the halo C1-C6 alkyl may be perhalo C1-C6 alkyl, and preferably, perfluoro C1-C6 alkyl.

According to an exemplary embodiment of the present invention, the diazaspiro compound of Chemical Formula 4 may have a symmetrical structure.

According to an exemplary embodiment of the present invention, the diazaspiro compound of Chemical Formula 4 may be selected from the following compounds, but is not limited thereto:

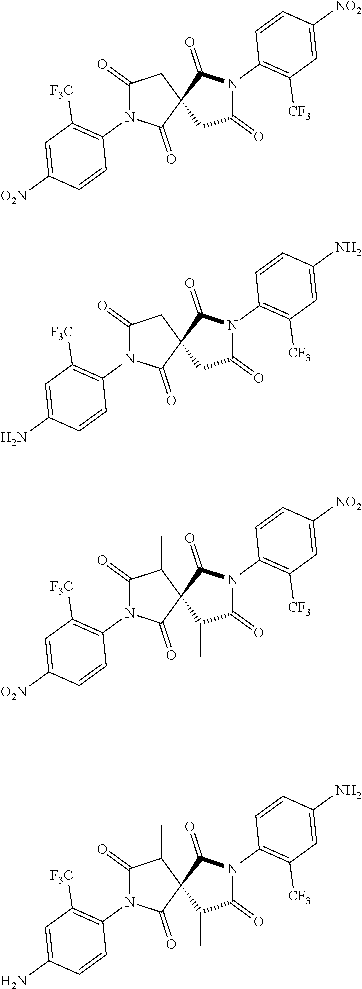

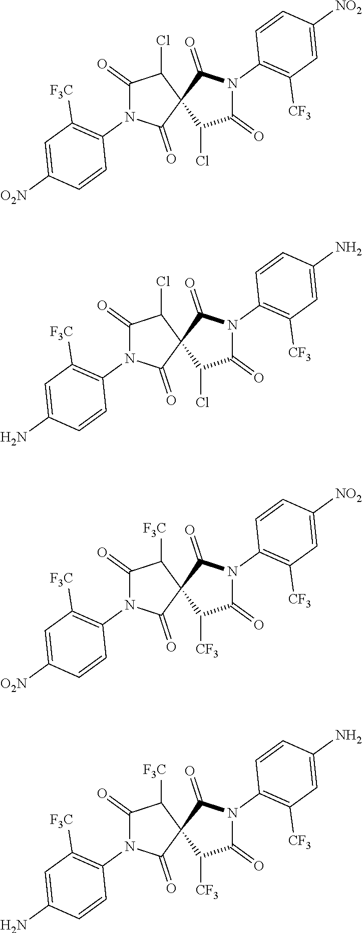

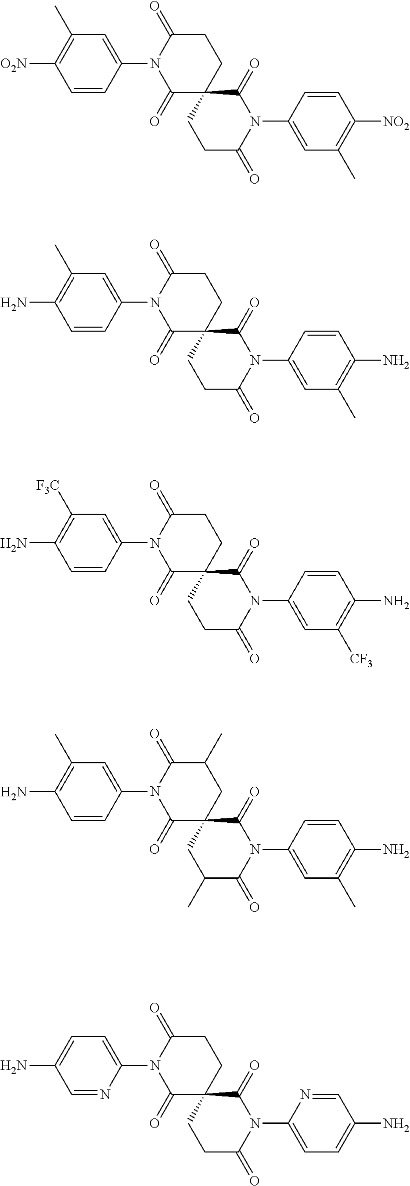

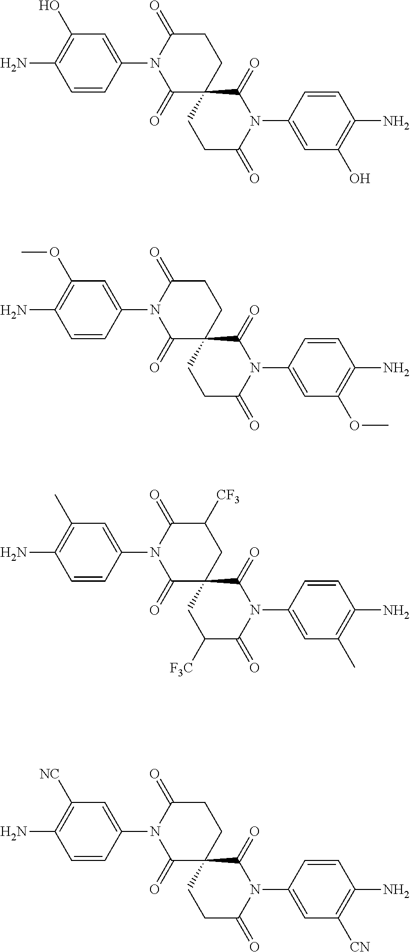

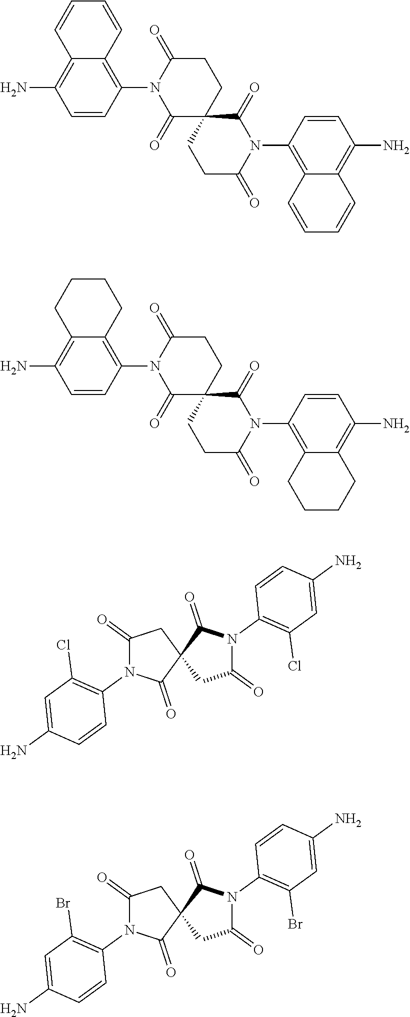

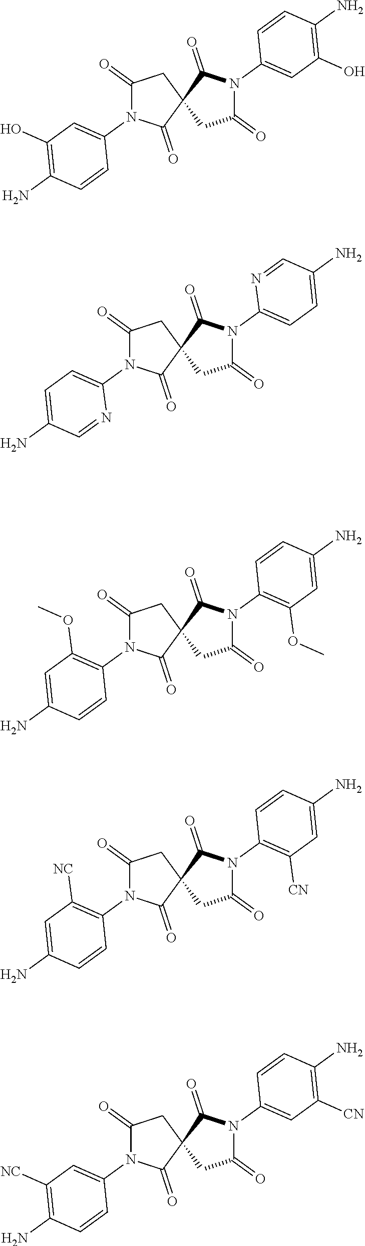

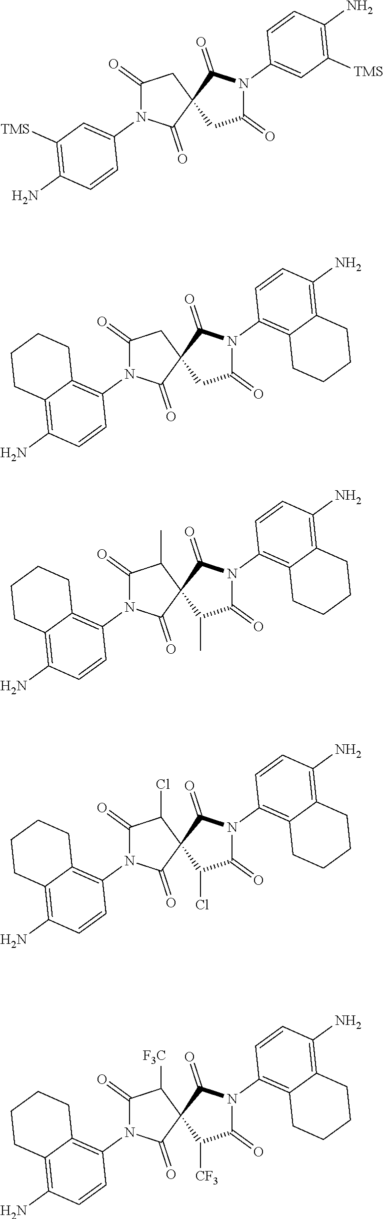

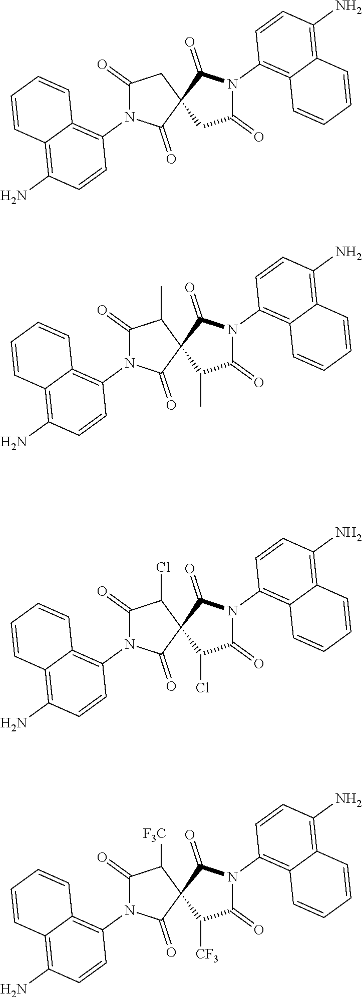

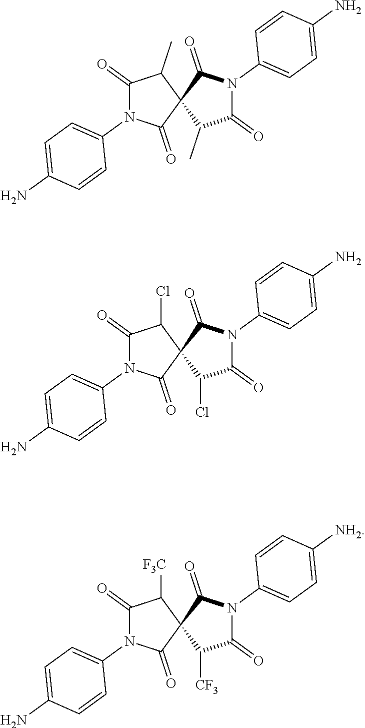

In terms of a use as a monomer for preparing polyimide having further improved physical properties, according to an exemplary embodiment of the present invention, Chemical Formula 1 may be represented by the following Chemical Formula 1-1 or 1-2:

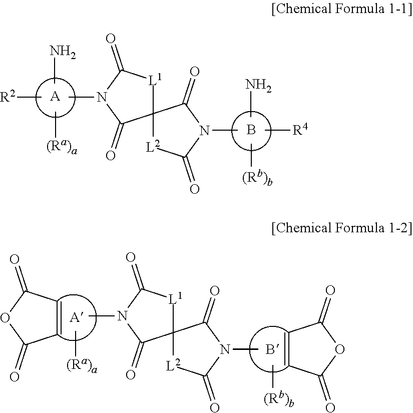

in Chemical Formulas 1-1 and 1-2,

a ring A, a ring B, a ring A′, and a ring B′ are each independently a C6-C20 aromatic ring or a C3-C20 heteroaromatic ring;

L¹ and L² are each independently substituted or unsubstituted hydrocarbylene;

R² and R⁴ are each independently hydrogen, halogen, substituted or unsubstituted hydrocarbyl, —OR^{b11}, —NR^{b12}R^{b13}, —COR^{b14}, —COOR^{b15}, nitro, —SiR^{b16}R^{b17}R^{b18}, or cyano;

R^{a} and R^{b} are each independently halogen, substituted or unsubstituted hydrocarbyl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring;

R^{b11} to R^{b18} and R^{c11} to R^{c18} are each independently hydrogen or substituted or unsubstituted hydrocarbyl; and

a and b are each independently an integer of 0 to 2, when a is an integer of 2, each of the R^{a}(s) may be the same as or different from each other, and when b is an integer of 2, each of the R^{b}(s) may be the same as or different from each other.

According to an exemplary embodiment of the present invention, Chemical Formula 1-1 may be represented by the following Chemical Formula 1-1A:

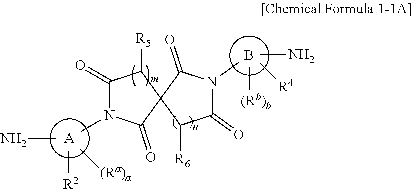

in Chemical Formula 1-1A, a ring A, a ring B, R², R⁴, R^{a}, R^{b}, a, and b are the same as defined in Chemical Formula 1-1;

R⁵ and R⁶ are each independently hydrogen, halogen, C1-C10 alkyl, or halo C1-C10 alkyl; and

m and n are each independently an integer of 1 to 5.

In an exemplary embodiment, R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano; R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring; R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl; R^{b17}, R^{b18}, R^{c17}, and R^{c18} are each independently C1-C10 alkyl, C3-C10 cycloalkyl, or C6-C20 aryl; and a and b may be each independently an integer of 0 or 1.

According to an exemplary embodiment of the present invention, Chemical Formula 1-1A may be represented by the following Chemical Formula 1-1B:

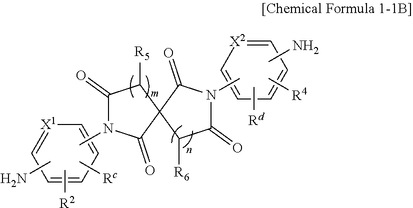

in Chemical Formula 1-1B,

each of X¹ and X² is CR or N;

R², R⁴, R, R^{c}, and R^{d} are each independently hydrogen, halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano, and R² and R^{c}, and R⁴ and R^{d} may be linked to each other by C3-C5 alkylene or C3-C5 alkenylene to form a fused ring;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl; and

m and n are each independently an integer of 1 to 3.

As a specific example, the halo C1-C10 alkyl may be perhalo C1-C10 alkyl, and preferably, perfluoro C1-C10 alkyl.

According to an exemplary embodiment of the present invention, Chemical Formula 1-1B may be represented by the following Chemical Formula 1-1C:

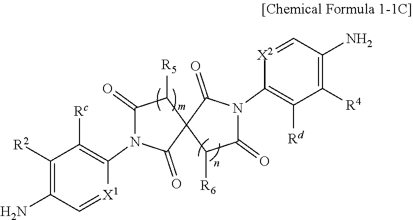

in Chemical Formula 1-1C,

each of X¹ and X² is CR or N;

R², R⁴, R, Re, and R^{d} are each independently hydrogen, halogen, C1-C6 alkyl, halo C1-C6 alkyl, hydroxy, C1-C6 alkoxy, —SiR^{e1}R^{e2}R^{e3}, or cyano, and R² and R^{c}, and R⁴ and R^{d} may be linked to each other by

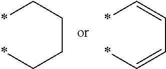

to form a fused ring;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl; and

m and n are each independently an integer of 1 or 2.

According to an exemplary embodiment of the present invention, Chemical Formula 1-2 may be represented by the following Chemical Formula 1-2A:

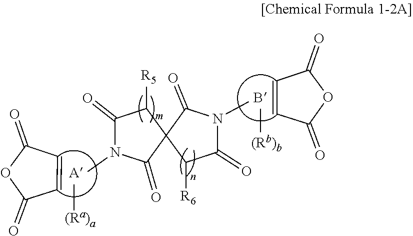

in Chemical Formula 1-2A, a ring A′, a ring B′, R^{a}, R^{b}, a, and b are the same as defined in Chemical Formula 1-2;

R⁵ and R⁶ are each independently hydrogen, halogen, C1-C10 alkyl, or halo C1-C10 alkyl; and

m and n are each independently an integer of 1 to 5.

In an exemplary embodiment of the present invention, the ring A′ and the ring B′ may be each independently benzene, naphthalene, or pyridine, and preferably, the ring A′ and the ring B′ may be the same as each other.

In an exemplary embodiment, R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano; R^{c11}, R^{c12}, R^{c13}, R^{c15}, and R^{c16} are each independently hydrogen, C1-C10 alkyl, C3-C10 cycloalkyl, or C6-C20 aryl; R^{c17} and R^{c18} are each independently C1-C10 alkyl, C3-C10 cycloalkyl, or C6-C20 aryl; and a and b may be each independently an integer of 0 or 1.

Specifically, in Chemical Formula 1-2A,

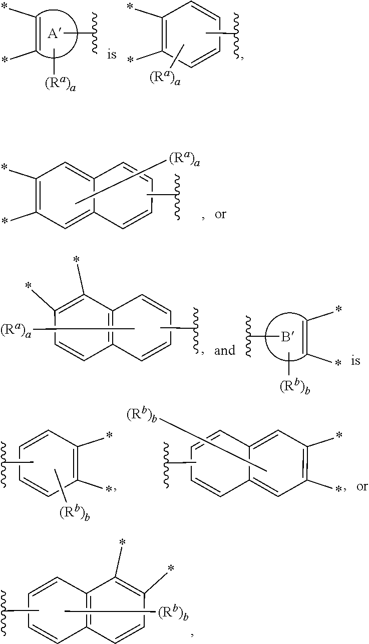

where a wavy line [symbol] represents a bonding site to a nitrogen atom, and an asterisk (*) represents a bonding site to a carbon atom; R^{a} and R^{b} are each independently halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano; R^{e1} is hydrogen, C1-C10 alkyl, C3-C10 cycloalkyl, or C6-C20 aryl; R^{e2} and R^{e3} are each independently C1-C10 alkyl, C3-C10 cycloalkyl, or C6-C20 aryl; a and b are each independently an integer of 0 to 2; and m and n may be each independently an integer of 1 to 3.

Preferably, according to an exemplary embodiment, Chemical Formula 1-2A may be represented by the following Chemical Formula 1-2B, 1-2C, or 1-2D:

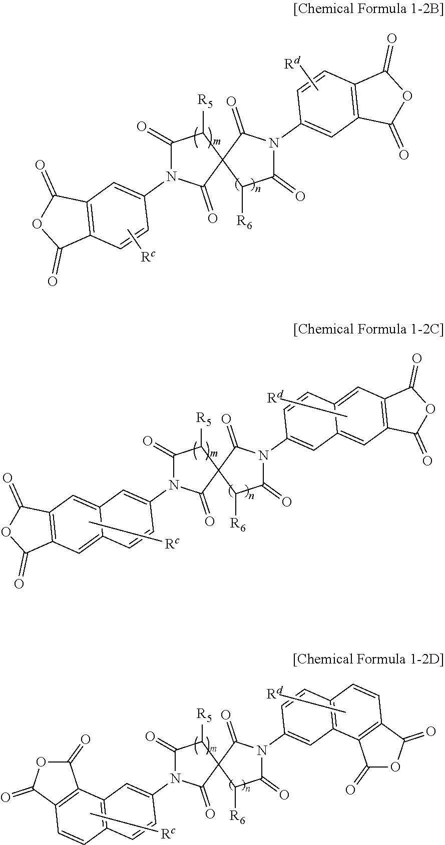

in Chemical Formula 1-2B, 1-2C, or 1-2D,

R⁵ and R⁶ are each independently hydrogen, halogen, C1-C6 alkyl, or halo C1-C6 alkyl;

R^{c} and R^{d} are each independently hydrogen, halogen, C1-C6 alkyl, halo C1-C6 alkyl, hydroxy, C1-C6 alkoxy, —SiR^{e1}R^{e2}R^{e3}, or cyano;

R^{e1} is hydrogen or C1-C6 alkyl;

R^{e2} and R^{e3} are each independently C1-C6 alkyl; and

m and n are each independently an integer of 1 or 2.

As a specific example, the halo C1-C6 alkyl may be perhalo C1-C6 alkyl, and preferably, perfluoro C1-C6 alkyl.

According to the present invention, the diazaspiro compound in the form of a diamine of Chemical Formula 1-1 or an acid dianhydride of Chemical Formula 1-2 with a central skeleton of diazaspiro tetrone, such that the diazaspiro compound is significantly useful as a monomer for synthesizing a polyimide-based polymer, and thus, the diazaspiro compound has excellent optical properties (a haze, a light transmittance, a yellow index, and the like) and improved Young's modulus properties. Therefore, it is possible to prepare polyimide having excellent flexibility. In addition, it is possible to prepare polyimide having no problems such as warping, peeling, and breaking and having a uniform light transmittance and transparency even during a heat treatment performed in the preparation of polyimide.

According to an exemplary embodiment of the present invention, specifically, the diazaspiro compound in the form of a diamine of Chemical Formula 1-1 or an acid dianhydride of Chemical Formula 1-2 may be selected from the following compounds, but is not limited thereto:

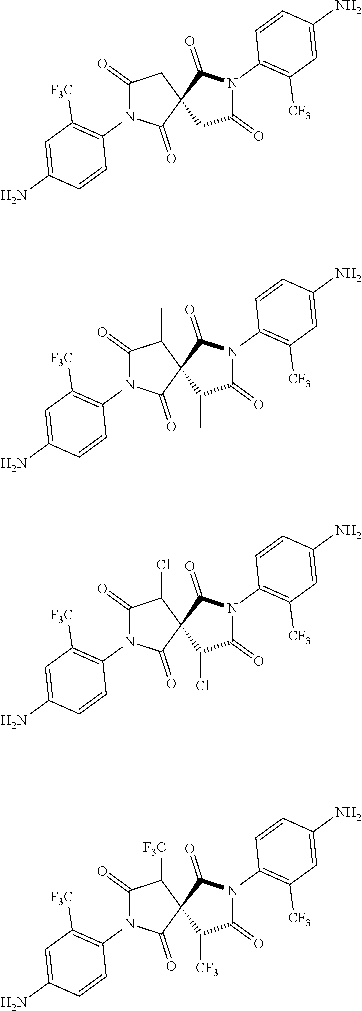

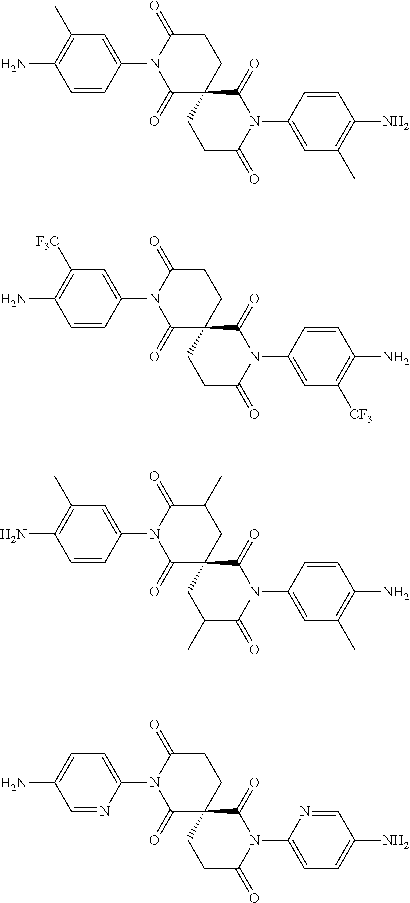

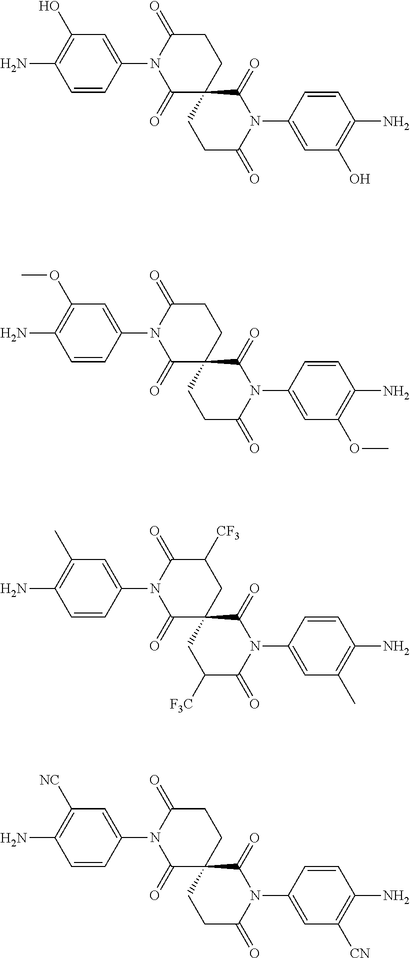

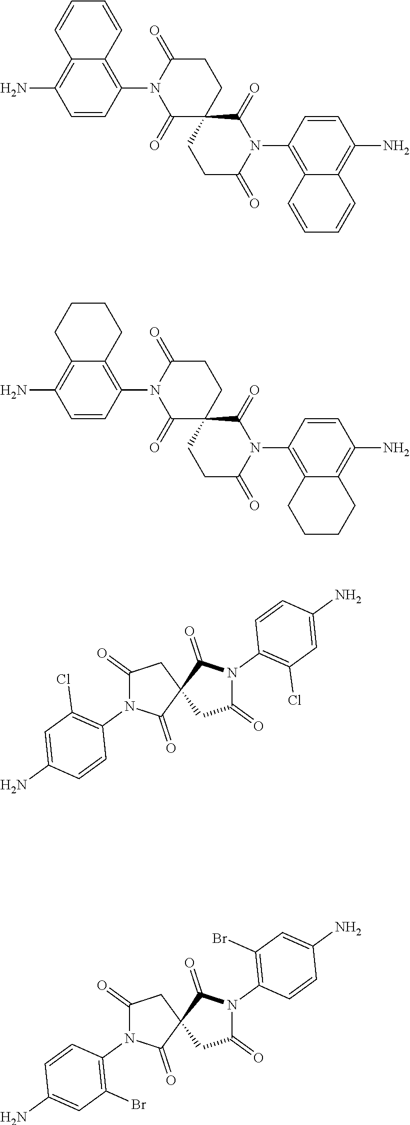

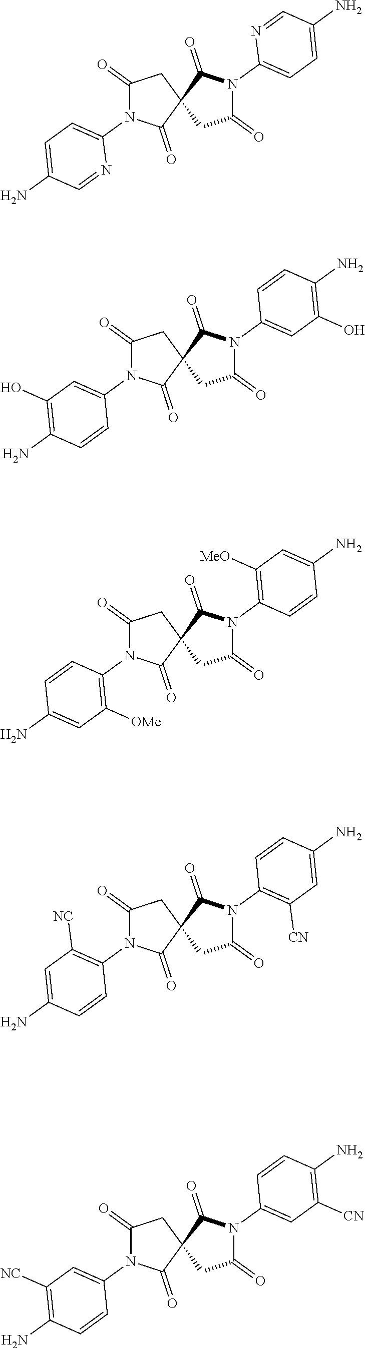

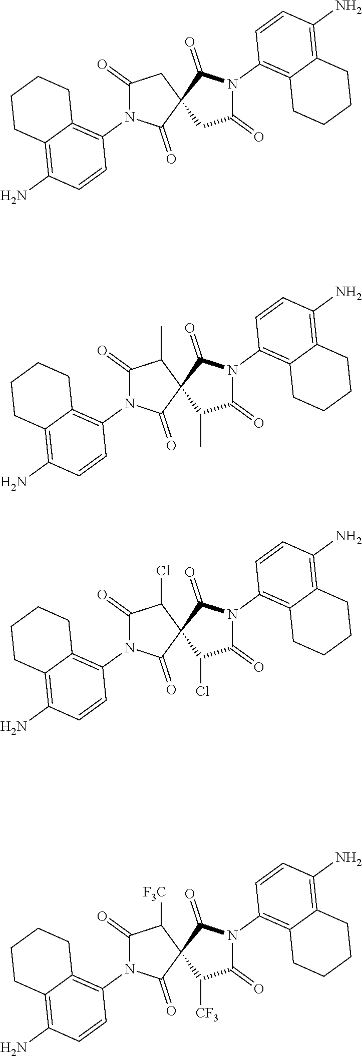

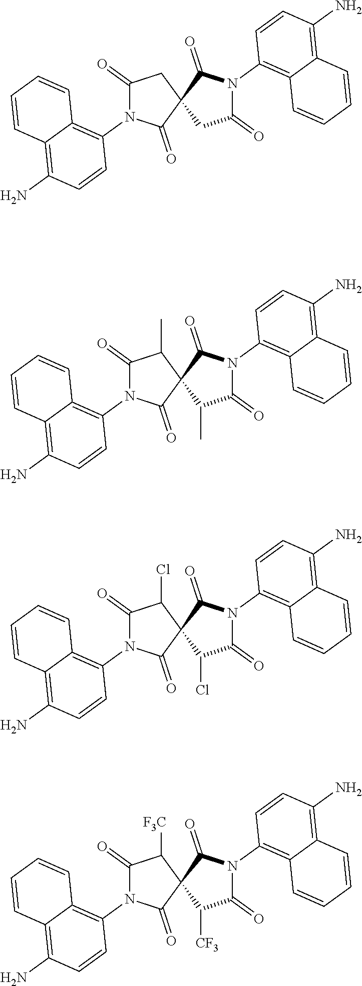

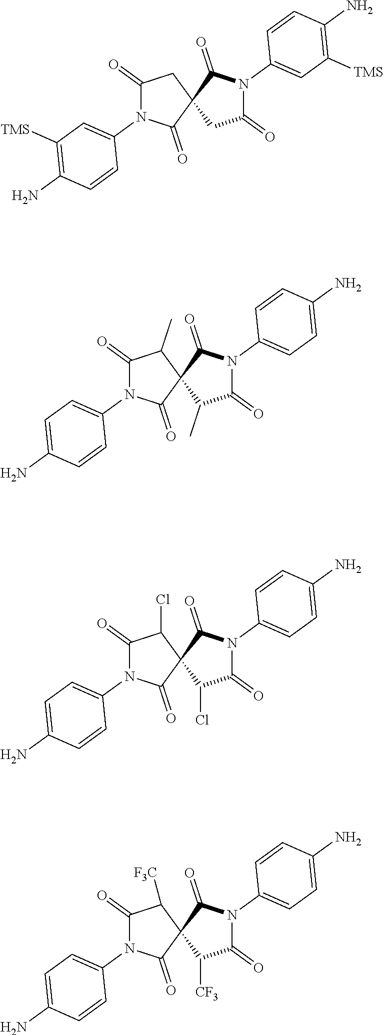

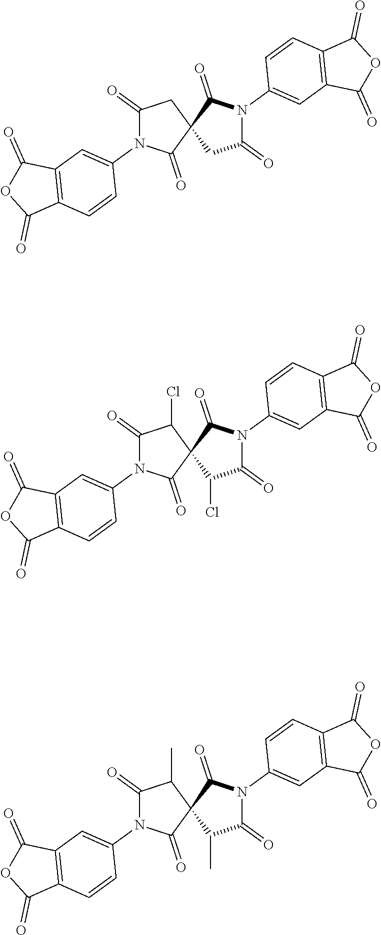

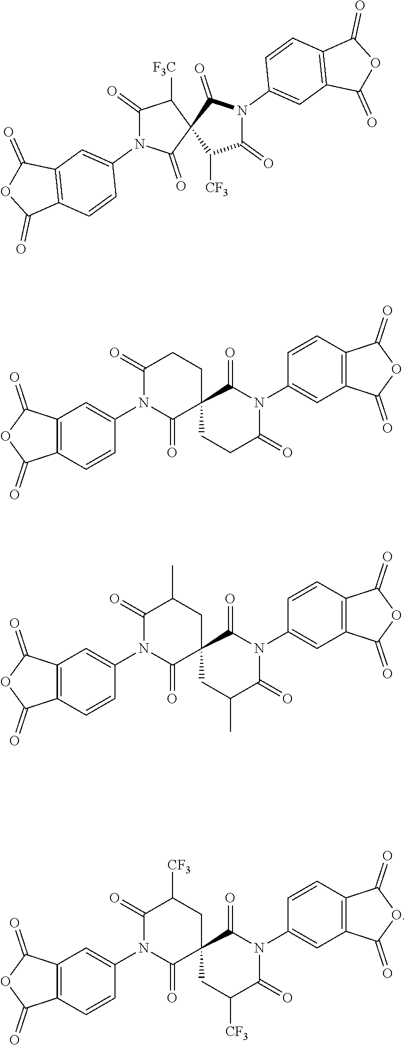

According to an exemplary embodiment of the present invention, the diazaspiro compound in the form of a diamine of Chemical Formula 1-1 may synthesize a polyimide precursor (that is, a polyamic acid or a polyamic acid ester) by a reaction with a known acid dianhydride compound, and may synthesize polyimide by imidization of a polyimide precursor, as described below. In addition, according to an exemplary embodiment of the present invention, the diazaspiro compound in the form of an acid dianhydride of Chemical Formula 1-2 may synthesize a polyimide precursor (that is, a polyamic acid or a polyamic acid ester) by a reaction with a known diamine compound, and may synthesize polyimide by imidization of a polyimide precursor, as described below.

That is, according to an exemplary embodiment, the diazaspiro compound in the form of a diamine of Chemical Formula 1-1 or an acid dianhydride of Chemical Formula 1-2 may be applied as a monomer for synthesizing a polyimide-based polymer. In this case, the polyimide-based polymer refers to both polyimide and a polyimide precursor (that is, a polyamic acid or a polyamic acid ester).

In addition, the present invention provides a polyimide precursor prepared by using the diazaspiro compound in the form of a diamine of Chemical Formula 1-1 or an acid dianhydride of Chemical Formula 1-2 as a monomer. According to an exemplary embodiment, the polyimide precursor has a structural unit derived from the diazaspiro compound in the form of a diamine of Chemical Formula 1-1 or a structural unit derived from the diazaspiro compound in the form of an acid dianhydride of Chemical Formula 1-2.

According to the present invention, the polyimide precursor has a structural unit derived from the diazaspiro compound in the form of a diamine of Chemical Formula 1-1 and a structural unit derived from a known acid dianhydride compound.

According to the present invention, the polyimide precursor has a structural unit derived from the diazaspiro compound in the form of an acid dianhydride of Chemical Formula 1-2 and a structural unit derived from a known diamine compound.

That is, the polyimide precursor may have a repeating unit in which a bond between the nitrogen atom of the amino group and the carbon atom of the anhydride group is formed by a reaction of the terminal amino group (—NH₂) of the diamine compound and the terminal anhydride group (—OC—O—CO—) of the acid dianhydride compound.

According to the present invention, the polyimide precursor has a monomer having a specific structure with a central skeleton of diazaspiro tetrone, that is, a structural unit derived from the diazaspiro compound in the form of a diamine of Chemical Formula 1-1 or an acid dianhydride of Chemical Formula 1-2, such that polyimide prepared by a ring closure reaction of the polyimide precursor may have improved Young's modulus properties in addition to excellent heat resistance and optical properties.

According to an exemplary embodiment, the polyimide precursor may further have a structural unit derived from a known diamine compound. As an example, the known diamine compound is a diamine compound commonly used for preparing a polyimide-based polymer, and is not particularly limited. Examples of the diamine compound include 4,4′-diaminodiphenyl propane, 4,4′-diaminodiphenyl methane, 4,4′-diaminodiphenyl sulfide, 3,3′-diaminodiphenyl sulfone, 4,4′-diaminodiphenyl sulfone, 4,4′-oxydianiline, 3,3′-oxydianiline, 3,4′-oxydianiline, 4,4′-diaminodiphenyl diethylsilane, 4,4′-diaminodiphenyl silane, 4,4′-diaminodiphenyl ethylphosphine oxide, 4,4′-diaminodiphenyl N-methylamine, 4,4′-diaminodiphenyl N-phenylamine, 1,4-diaminobenzene(p-phenylenediamine), bis{4-(4-aminophenoxy)phenyl}sulfone, bis{4-(3-aminophenoxy)phenyl}sulfone, 4,4′-bis(4-aminophenoxy)biphenyl, 4,4′-bis(3-aminophenoxy)biphenyl, 1,3-bis(3-aminophenoxy)benzene, 1,3-bis(4-aminophenoxy)benzene, 3,3′-diaminobenzophenone, 4,4′-diaminobenzophenone, and 2,2-bis(4-aminophenoxyphenyl)propane, and these diamine compounds may be used alone or in combination thereof, but the present invention is not limited thereto.

According to an exemplary embodiment, the polyimide precursor may further have a structural unit derived from a diamine compound represented by the following Chemical Formula E:

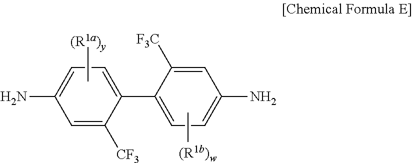

in Chemical Formula E,

R^{1a} and R^{1b} are each independently halogen, C1-C10 alkyl, halo C1-C10 alkyl, C1-C10 alkoxy, or C6-C12 aryl; and

y and w are each independently an integer of 0 to 3.

Preferably, in Chemical Formula E, R^{1a} and R^{1b} are each independently halogen, C1-C6 alkyl or halo C1-C6 alkyl; and y and w may be each independently an integer of 0 or 1, and more preferably, each of y and w may be 0.

In an exemplary embodiment, when a polyimide precursor is prepared by further including a known diamine compound additionally included as a monomer in addition to the diazaspiro compound in the form of a diamine of Chemical Formula 1-1 as a diamine monomer, a ratio of the diazaspiro compound in the form of a diamine of Chemical Formula 1-1 as a monomer may be 0.1 to 99 mol %, preferably, 10 to 90 mol %, and more preferably, 10 to 80 mol %, with respect to the total content of the diazaspiro compound in the form of a diamine of Chemical Formula 1-1 and the known diamine compound, specifically, the diamine compound of Chemical Formula E, but is not limited thereto.

In an exemplary embodiment of the present invention, any acid dianhydride compound may be used as long as it is a compound having a functional group, and specifically, the acid dianhydride compound may be a tetracarboxylic dianhydride. The tetracarboxylic dianhydride may be at least one selected from C8-C36 aromatic tetracarboxylic dianhydride, C6-C50 aliphatic tetracarboxylic dianhydride, and C6-C36 alicyclic tetracarboxylic dianhydride. That is, the tetracarboxylic acid compounds may be used alone or in combination of two or more thereof. In terms of having an excellent yellow index even in a high temperature range, the tetracarboxylic acid compound may be preferably C8-C36 aromatic tetracarboxylic dianhydride. According to an exemplary embodiment, the number of carbon atoms in the tetracarboxylic dianhydride includes the number of carbon atoms contained in a carboxyl group.

Specific examples of the C8-C36 aromatic tetracarboxylic dianhydride include 4,4′-(hexafluoroisopropylidene)diphthalic anhydride (6FDA), 5-(2,5-dioxotetrahydro-3-furanyl)-3-methyl-cyclohexene-1,2-dicarboxylic anhydride, pyromellitic dianhydride, 1,2,3,4-benzenetetracarboxylic dianhydride, 3,3′,4,4′-benzophenonetetracarboxylic dianhydride, 2,2′,3,3′-benzophenonetetracarboxylic dianhydride, 3,3′,4,4′-biphenyltetracarboxylic dianhydride, 3,3′,4,4′-diphenylsulfonetetracarboxylic dianhydride, 2,2′,3,3′-biphenyltetracarboxylic dianhydride, methylene-4,4′-diphthalic dianhydride, 1,1-ethylidene-4,4′-diphthalic dianhydride, 2,2-propylidene-4,4′-diphthalic dianhydride, 1,2-ethylene-4,4′-diphthalic dianhydride, 1,3-trimethylene-4,4′-diphthalic dianhydride, 1,4-tetramethylene-4,4′-diphthalic dianhydride, 1,5-pentamethylene-4,4′-diphthalic dianhydride, 4,4′-oxydiphthalic dianhydride, p-phenylenebis(trimellitate anhydride), thio-4,4′-diphthalic dianhydride, sulfonyl-4,4′-diphthalic dianhydride, 1,3-bis(3,4-dicarboxyphenyl)benzene dianhydride, 1,3-bis(3,4-dicarboxyphenoxy)benzene dianhydride, 1,4-bis(3,4-dicarboxyphenoxy)benzene dianhydride, 1,3-bis[2-(3,4-dicarboxyphenyl)-2-propyl]benzene dianhydride, 1,4-bis[2-(3,4-dicarboxyphenyl)-2-propyl]benzene dianhydride, bis[3-(3,4-dicarboxyphenoxy)phenyl]methane dianhydride, bis[4-(3,4-dicarboxyphenoxy)phenyl]methane dianhydride, 2,2-bis[3-(3,4-dicarboxyphenoxy)phenyl]propane dianhydride, 2,2-bis[4-(3,4-dicarboxyphenoxy)phenyl]propane dianhydride, bis(3,4-dicarboxyphenoxy)dimethylsilane dianhydride, 1,3-bis(3,4-dicarboxyphenyl)-1,1,3,3-tetramethyldisiloxane dianhydride, 2,3,6,7-naphthalenetetracarboxylic dianhydride, 1,4,5,8-naphthalenetetracarboxylic dianhydride, 1,2,5,6-naphthalenetetracarboxylic dianhydride, 3,4,9,10-perylenetetracarboxylic dianhydride, 2,3,6,7-anthracenetetracarboxylic dianhydride, and 1,2,7,8-phenanthrenetetracarboxylic dianhydride. Specific examples of the C6-C50 aliphatic tetracarboxylic dianhydride include ethylenetetracarboxylic dianhydride, 1,2,3,4-butanetetracarboxylic dianhydride, and 1,2,3,4-pentanetetracarboxylic dianhydride. Specific examples of the C6-C36 alicyclic tetracarboxylic dianhydride include 1,2,3,4-cyclobutanetetracarboxylic dianhydride, cyclopentanetetracarboxylic dianhydride, cyclohexane-1,2,3,4-tetracarboxylic dianhydride, cyclohexane-1,2,4,5-tetracarboxylic dianhydride, 3,3′,4,4′-bicyclohexyltetracarboxylic dianhydride, carbonyl-4,4′-bis(cyclohexane-1,2-dicarboxylic) dianhydride, methylene-4,4′-bis(cyclohexane-1,2-dicarboxylic) dianhydride, 1,2-ethylene-4,4′-bis(cyclohexane-1,2-dicarboxylic) dianhydride, 1,1-ethylidene-4,4′-bis(cyclohexane-1,2-dicarboxylic) dianhydride, 2,2-propylidene-4,4′-bis(cyclohexane-1,2-dicarboxylic) dianhydride, oxy-4,4′-bis(cyclohexane-1,2-dicarboxylic) dianhydride, thio-4,4′-bis(cyclohexane-1,2-dicarboxylic) dianhydride, sulfonyl-4,4′-bis(cyclohexane-1,2-dicarboxylic) dianhydride, bicyclo[2,2,2]oct-7-ene-2,3,5,6-tetracarboxylic dianhydride, 4-(2,5-dioxotetrahydrofuran-3-yl)-1,2,3,4-tetrahydronaphthalene-1,2-dicarboxylic anhydride, and ethylene glycol-bis-(3,4-dicarboxylic anhydride phenyl)ether. These compounds may be used alone or in combination of two or more thereof.

In terms of implementing excellent chemical resistance and yellow index, according to an exemplary embodiment, the acid dianhydride compound may be more preferably an acid dianhydride represented by the following Chemical Formula D:

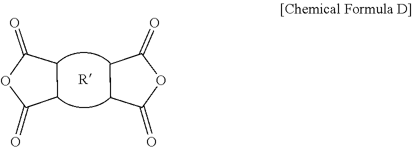 [SMILES: O=C1CCC(=O)O1 ; O=C1CCC(=O)O1]

in Chemical Formula D,

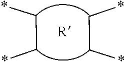

is at least one tetravalent group selected from

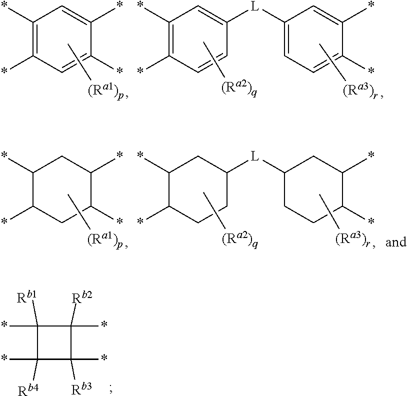

R^{a1}, R^{a2}, and R^{a3} are each independently C1-C10 alkyl or halo C1-C10 alkyl;

L is a single bond, C1-C10 alkylene, —O—, —S—, —CO—, —SO₂—, —SiR^{f1}R^{f2}—, —CO—Ar—CO—, or —O—Ar¹—(X—Ar²)ₛ—O—, and the alkylene may be further substituted with one or more selected from C1-C10 alkyl and halo C1-C10 alkyl;

R^{f1} and R^{f2} are each independently C1-C10 alkyl;

Ar, Ar¹, and Ar² are each independently C6-C20 arylene, and the arylene may be further substituted with one or more selected from C1-C10 alkyl and halo C1-C10 alkyl;

X is O or S;

R^{b1} to R^{b4} are each independently hydrogen, C1-C10 alkyl, or C6-C20 aryl;

p, q, and r are each independently an integer of 0 to 2; and

s is an integer of 0 or 1.

According to an exemplary embodiment, in Chemical Formula D,

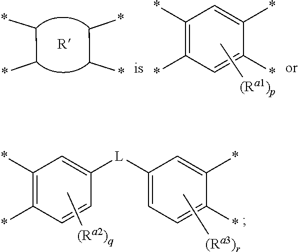

R^{a1}, R^{a2}, and R^{a3} are each independently C1-C10 alkyl or halo C1-C10 alkyl; L is a single bond, C1-C10 alkylene, —O—, —S—, —CO—, or —SO₂—, and the alkylene may be further substituted with one or more selected from C1-C10 alkyl and halo C1-C10 alkyl; and p, q, and r may be each independently an integer of 0 or 1.

Preferably, according to an exemplary embodiment, in Chemical Formula D,

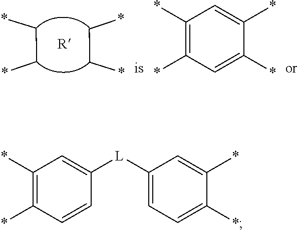

L is a single bond or —CR^{c1}R^{c2}—; and R^{c1} and R^{c2} may be each independently C1-C10 alkyl or halo C1-C10 alkyl.

More preferably, according to an exemplary embodiment, in Chemical Formula D,

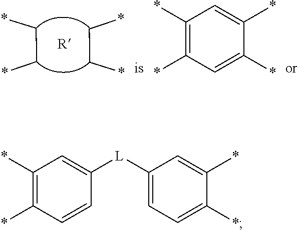

L is a single bond or —CR^{c1}R^{c2}—; R^{c1} and R^{c2} are each independently C1-C6 alkyl or —C_{z}T¹_{2z+1}; T¹ is halogen; and z may be an integer of 1 to 6, and more preferably, R^{c1} and R^{c2} may be each independently CH₃ or CF₃.

According to an exemplary embodiment, the polyimide precursor may have a repeating unit represented by the following Chemical Formula 11:

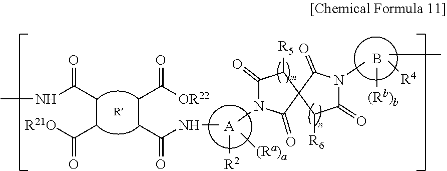

in Chemical Formula 11,

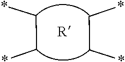

is the same as defined in Chemical Formula D;

R²¹ and R²² are each independently hydrogen or C1-C10 alkyl;

a ring A and a ring B are each independently a C6-C20 aromatic ring or a C3-C20 heteroaromatic ring;

R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;

R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;

R^{b17}, R^{b18}, R^{c17}, and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1; and

m and n are each independently an integer of 1 to 5.

According to an exemplary embodiment, in Chemical Formula 11,

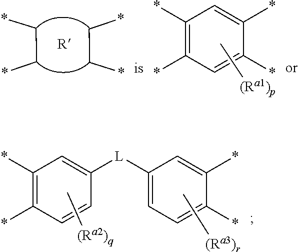

R^{a1}, R^{a2}, and R^{a3} are each independently C1-C10 alkyl or halo C1-C10 alkyl; R²¹ and R²² are each independently hydrogen or C1-C10 alkyl; L is a single bond, C1-C10 alkylene, —O—, —S—, —CO—, or —SO₂—, and the alkylene may be further substituted with one or more selected from C1-C10 alkyl and halo C1-C10 alkyl; and p, q, and r may be each independently an integer of 0 or 1.

According to an exemplary embodiment, the polyimide precursor may have a repeating unit represented by the following Chemical Formula 11-1:

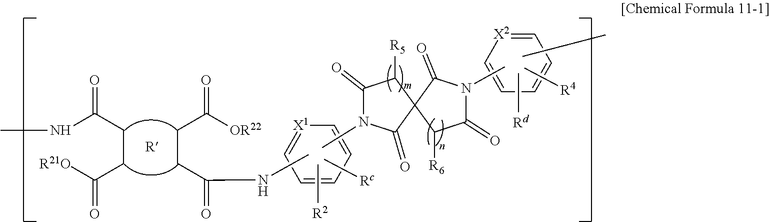

in Chemical Formula 11-1,

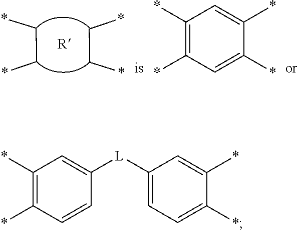

R²¹ and R²² are each independently hydrogen or C1-C10 alkyl;

L is a single bond or —CR^{c1}R^{c2}—;

R^{c1} and R^{c2} are each independently C1-C10 alkyl or halo C1-C10 alkyl;

each of X¹ and X² is CR or N;

R², R⁴, R, R^{c}, and R^{d} are each independently hydrogen, halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano, and R² and R^{c}, and R⁴ and R^{d} may be linked to each other by C3-C5 alkylene or C3-C5 alkenylene to form a fused ring;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl; and

m and n are each independently an integer of 1 to 3.

More preferably, according to an exemplary embodiment, the polyimide precursor may have a repeating unit represented by the following Chemical Formula 11-2:

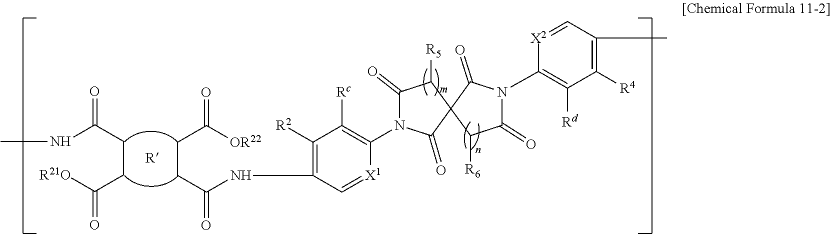

in Chemical Formula 11-2,

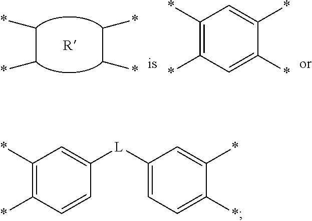

R²¹ and R²² are each independently hydrogen or C1-C10 alkyl;

L is a single bond or —CR^{c1}R^{c2}—;

R^{c1} and R^{c2} are each independently C1-C6 alkyl or —C_{z}T¹_{2z+1};

each of X¹ and X² is CR or N;

R², R⁴, R, R^{c}, and R^{d} are each independently hydrogen, halogen, C1-C6 alkyl, —C_{z′}T²_{2z′+1}, hydroxy, C1-C6 alkoxy, —SiR^{e1}R^{e2}R^{e3}, or cyano, and R² and R^{c}, and R⁴ and R^{d} may be linked to each other by

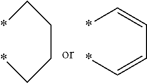

to form a fused ring;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or —C_{z″}T³_{2z″+1};

T¹, T², and T³ are each independently halogen;

z, z′, and z″ are each independently an integer of 1 to 6; and

m and n are each independently an integer of 1 or 2.

Specifically, according to an exemplary embodiment, in Chemical Formula 11-2,

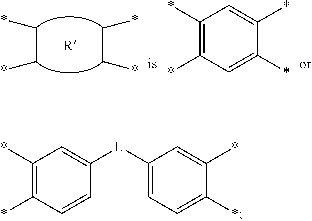

L is a single bond or —CR^{c1}R^{c2}—; R^{c1} and R^{c2} are each independently C1-C6 alkyl or —C_{z}T¹_{2z+1}; T¹ is halogen; and z may be an integer of 1 to 6, and more preferably, R^{c1} and R^{c2} may be each independently CH₃ or CF₃.

According to an exemplary embodiment, the polyimide precursor may have a repeating unit represented by the following Chemical Formula 12:

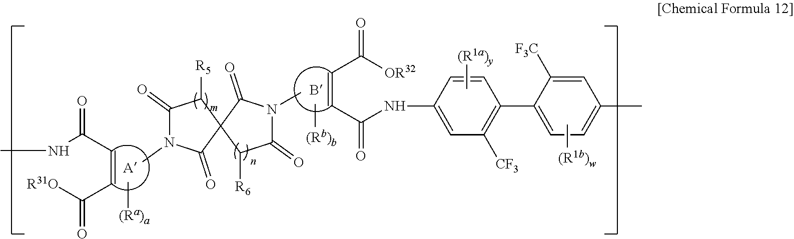

in Chemical Formula 12, R^{1a}, R^{1b}, y, and w are the same as defined in Chemical Formula E;

R³¹ and R³² are each independently hydrogen or C1-C10 alkyl;

a ring A′ and a ring B′ are each independently a C6-C20 aromatic ring or a C3-C20 heteroaromatic ring;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;

R^{c11}, R^{c12}, R^{c13}, R^{c15}, and R^{c16} are each independently hydrogen, C1-C10 alkyl, C3-C10 cycloalkyl, or C6-C20 aryl;

R^{c17} and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1; and

m and n are each independently an integer of 1 to 5.

According to an exemplary embodiment, the polyimide precursor may have a repeating unit represented by the following Chemical Formula 12-1:

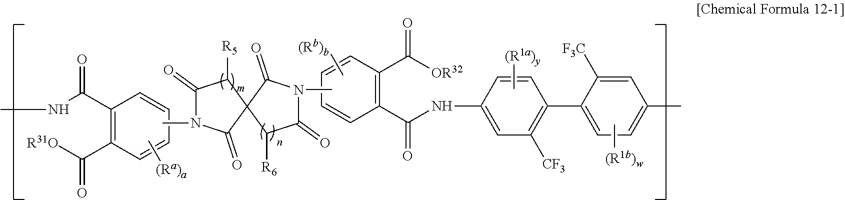

in Chemical Formula 12-1,

R³¹ and R³² are each independently hydrogen or C1-C10 alkyl;

R^{a} and R^{b} are each independently halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

a and b are each independently an integer of 0 to 2;

R⁵ and R⁶ are each independently hydrogen, halogen, C1-C10 alkyl, or halo C1-C10 alkyl; and

m and n may be each independently an integer of 1 to 3.

According to an exemplary embodiment, the polyimide precursor may have a repeating unit represented by the following Chemical Formula 12-2:

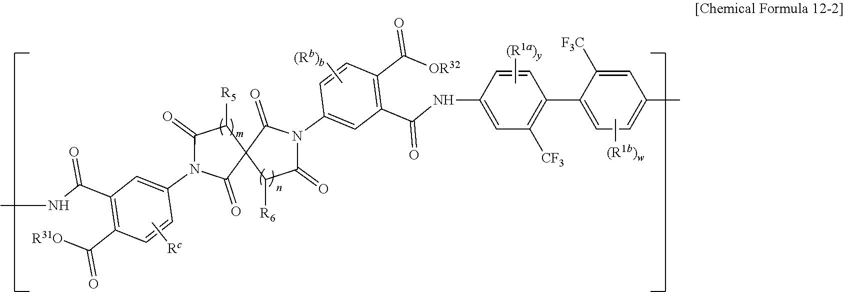

in Chemical Formula 12-2,

R^{1a} and R^{1b} are each independently halogen, C1-C6 alkyl, or halo C1-C6 alkyl;

R³¹ and R³² are each independently hydrogen or C1-C10 alkyl;

R^{c} and R^{d} are each independently hydrogen, halogen, C1-C6 alkyl, halo C1-C6 alkyl, hydroxy, C1-C6 alkoxy, —SiR^{e1}R^{e2}R^{e3}, or cyano;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl;

m and n are each independently an integer of 1 or 2; and

y and w are each independently an integer of 0 or 1.

According to an exemplary embodiment, the polyimide precursor may have a repeating unit represented by the following Chemical Formula F:

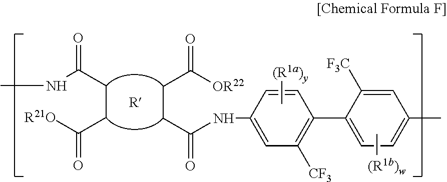

in Chemical Formula F,

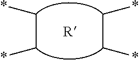

is the same as defined in Chemical Formula D;

R²¹ and R²² are each independently hydrogen or C1-C10 alkyl;

R^{1a} and R^{1b} are each independently halogen, C1-C10 alkyl, halo C1-C10 alkyl, C1-C10 alkoxy, or C6-C12 aryl; and

y and w are each independently an integer of 0 to 3.

According to an exemplary embodiment, in Chemical Formula F,

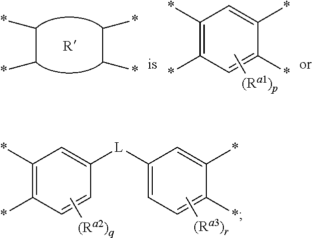

R^{a1}, R^{a2}, and R^{a3} are each independently C1-C6 alkyl or halo C1-C6 alkyl; R²¹ and R²² are each independently hydrogen or C1-C6 alkyl; L is a single bond, C1-C5 alkylene, —O—, —S—, —CO—, or —SO₂—, and the alkylene may be further substituted with one or more selected from C1-C6 alkyl and halo C1-C6 alkyl; R^{1a} and R^{1b} are each independently halogen, C1-C6 alkyl, or halo C1-C6 alkyl; y and w are each independently an integer of 0 or 1; and p, q, and r may be each independently an integer of 0 or 1.

Preferably, according to an exemplary embodiment, in Chemical Formula F,

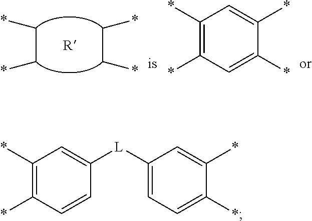

R²¹ and R²² are each independently hydrogen; L is a single bond or —CR^{c1}R^{c2}—; R^{c1} and R^{c2} are each independently C1-C3 alkyl or halo C1-C3 alkyl, and more preferably, R^{c1} and R^{c2} may be each independently CH₃ or CF₃; R^{1a} and R^{1b} are each independently halogen, C1-C5 alkyl, or halo C1-C5 alkyl; and y and w are each independently an integer of 0 or 1, and more preferably, each of y and w may be 0.

According to an exemplary embodiment, the polyimide precursor essentially has a repeating unit represented by Chemical Formula 11 or 12, and may further have a repeating unit represented by Chemical Formula F. Preferably, according to an exemplary embodiment, the polyimide precursor essentially has a repeating unit represented by Chemical Formula 11-1 or 12-1, and may further have a repeating unit represented by Chemical Formula F. More preferably, according to an exemplary embodiment, the polyimide precursor essentially has a repeating unit represented by Chemical Formula 11-2 or 12-2, and may further have a repeating unit represented by Chemical Formula F.

According to an exemplary embodiment, the repeating unit represented by Chemical Formula 11 or 12 may be included in the polyimide precursor in an amount of 10 to 100 mol %, preferably, 30 to 100 mol %, more preferably, 40 to 95 mol %, and still more preferably, 50 to 80 mol %.

According to an exemplary embodiment, the repeating unit represented by Chemical Formula F may be included in the polyimide precursor in an amount of 90 mol % or less, 70 mol % or less, 5 to 60 mol %, or 20 to 50 mol % with respect to a total of mol % of the polyimide precursor.

In addition, the present invention provides a polyimide-based polymer composition containing a diazaspiro compound in the form of a diamine of Chemical Formula 1-1 or an acid dianhydride of Chemical Formula 1-2.

The composition according to an exemplary embodiment may be a polyimide-based polymer composition having a structural unit derived from the diazaspiro compound in the form of a diamine of Chemical Formula 1-1 and a structural unit derived from a known acid dianhydride compound.

The composition according to an exemplary embodiment may be a polyimide-based polymer composition having a structural unit derived from the diazaspiro compound of Chemical Formula 1-1 and a structural unit derived from an acid dianhydride compound represented by Chemical Formula D.

The composition according to an exemplary embodiment may be a polyimide-based polymer composition having a repeating unit represented by the following Chemical Formula 13:

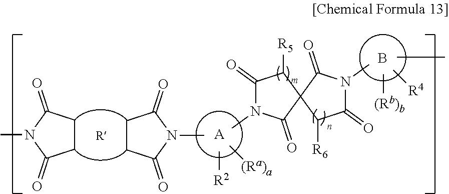

in Chemical Formula 13,

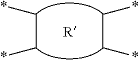

is the same as defined in Chemical Formula D;

a ring A and a ring B are each independently a C6-C20 aromatic ring or a C3-C20 heteroaromatic ring;

R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;

R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;

R^{b17}, R^{b18}, R^{c17}, and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1; and

m and n are each independently an integer of 1 to 5.

The composition according to an exemplary embodiment may be a polyimide-based polymer composition having a structural unit derived from the diazaspiro compound in the form of an acid dianhydride of Chemical Formula 1-2 and a structural unit derived from a known diamine compound.

The composition according to an exemplary embodiment may be a polyimide-based polymer composition having a structural unit derived from the diazaspiro compound of Chemical Formula 1-2 and a structural unit derived from a diamine compound represented by Chemical Formula E.

The composition according to an exemplary embodiment may be a polyimide-based polymer composition having a repeating unit represented by the following Chemical Formula 14:

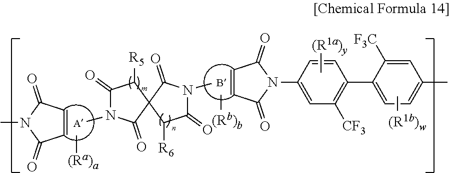

in Chemical Formula 14,

R^{1a}, R^{1b}, y, and w are the same as defined in Chemical Formula E;

a ring A′ and a ring B′ are each independently a C6-C20 aromatic ring or a C3-C20 heteroaromatic ring;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;

R^{c11}, R^{c12}, R^{c13}, R^{c15}, and R^{c16} are each independently hydrogen, C1-C10 alkyl, C3-C10 cycloalkyl, or C6-C20 aryl;

R^{c17} and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1; and

m and n are each independently an integer of 1 to 5.

The polyimide-based polymer composition contains both polyimide and a polyimide precursor (that is, a polyamic acid or a polyamic acid ester).

The composition according to an exemplary embodiment may be a polyimide precursor composition containing a polyimide precursor (that is, a polyamic acid or a polyamic acid ester) having a structural unit derived from the diazaspiro compound in the form of a diamine of Chemical Formula 1-1 and a structural unit derived from a known acid dianhydride compound, and an organic solvent.

The composition according to an exemplary embodiment may be a polyimide precursor composition containing a polyimide precursor (that is, a polyamic acid or a polyamic acid ester) having a structural unit derived from the diazaspiro compound in the form of an acid dianhydride of Chemical Formula 1-2 and a structural unit derived from a known diamine compound, and an organic solvent.

In an exemplary embodiment, the polyimide precursor composition may be in the form of a solution in which a polyimide precursor is dissolved in an organic solvent. For example, in a case where a polyimide precursor is synthesized in an organic solvent, a solution to be obtained may be a reaction solution itself or may be diluted with another solvent. In a case where a polyimide precursor is obtained as a solid powder, the polyimide precursor composition may be prepared as a solution by dissolving the polyimide precursor in an organic solvent.

In an exemplary embodiment, the polyimide precursor composition contains the polyimide precursor of the present invention described above, such that a polyimide film having significantly improved optical and mechanical properties may be implemented.

In particular, the polyimide precursor composition of the present invention may provide a polyimide film having high transparency, excellent heat resistance, and excellent elasticity.

According to an exemplary embodiment, the organic solvent in the polyimide precursor composition may be one or a mixture of two or more selected from ketones such as γ-butyrolactone, 1,3-dimethyl-2-imidazolidinone, methyl ethyl ketone, cyclohexanone, cyclopentanone, and 4-hydroxy-4-methyl-2-pentanone; aromatic hydrocarbons such as toluene, xylene, and tetramethylbenzene; glycol ethers (cellosolves) such as ethylene glycol monoethyl ether, ethylene glycol monomethyl ether, ethylene glycol monobutyl ether, diethylene glycol monoethyl ether, diethylene glycol monomethyl ether, diethylene glycol monobutyl ether, propylene glycol monomethyl ether, propylene glycol monoethyl ether, dipropylene glycol diethyl ether, and triethylene glycol monoethyl ether; acetates such as ethyl acetate, butyl acetate, ethylene glycol monoethyl ether acetate, ethylene glycol monobutyl ether acetate, diethylene glycol monoethyl ether acetate, and dipropylene glycol monomethyl ether acetate; alcohols such as methanol, ethanol, propanol, ethylene glycol, propylene glycol, and carbitol; and amides such as N,N-dimethylpropionamide (DMPA), N,N-diethylpropionamide (DEPA), N,N-dimethylacetamide (DMAc), N,N-diethylacetamide (DEAc), N,N-dimethylformamide (DMF), N,N-diethylformamide (DEF), N-methylpyrrolidone (NMP), N-ethylpyrrolidone (NEP), and N,N-dimethylmethoxy acetamide.

As an example, the organic solvent may be one or a mixture of two or more selected from the amides described above.

As an example, the organic solvent may be amides having a boiling point of 300° C. or lower. Specifically, the organic solvent may be N,N-diethylformamide (DEF), N,N-dimethylacetamide (DMAc), N,N-diethylacetamide (DEAc), N-ethylpyrrolidone (NEP), N,N-dimethylpropionamide (DMPA), N,N-diethylpropionamide (DEPA), or a combination thereof.

According to an exemplary embodiment, the polyimide precursor composition may contain a solid in an amount in which the composition has an appropriate viscosity in consideration of applicability during a film forming process, and a solid content may be 5 to 30 wt %, and preferably, 10 to 25 wt %, with respect to the total weight of the composition.

Specifically, according to an exemplary embodiment, the polyimide precursor composition may have a viscosity of 2,000 to 50,000 cPs. Specifically, the viscosity may be 30,000 cPs or less. When the viscosity is in the above range, the process efficiency may be excellent due to excellent defoamation efficiency when processing a polyimide film. As a result, a more uniform surface may be implemented, which is preferable. In this case, the viscosity means a value measured by placing a sample at room temperature (25° C.) using a Brookfield RVDV-III viscometer spindle No. 52 and stabilizing the sample for 2 minutes when a torque value reaches 80%.

According to an exemplary embodiment, the polyimide precursor composition may be prepared by polymerizing the diamine compound and the acid dianhydride compound according to an exemplary embodiment in the presence of an organic solvent. According to an exemplary embodiment, the diamine compound and the acid dianhydride compound may be polymerized at a molar ratio of 2:1 to 1:2, preferably, 1.5:1 to 1:1.5, and more preferably, 1:1.1 to 1.1:1. The molar ratio may vary depending on intended reactivity and processability.

According to an exemplary embodiment, the polymerization of the diamine compound and the acid dianhydride compound may be performed under an inert gas or a nitrogen stream, and may be performed under anhydrous conditions. In addition, the polymerization reaction may be performed at a reaction temperature of 80° C. or lower, −20 to 80° C., and preferably, 0 to 80° C. When the reaction temperature is too high, the reactivity may be increased, which causes an increase in molecular weight, and the viscosity of the precursor composition is increased, which may be disadvantageous in the process.

According to an exemplary embodiment, a molecular weight of the polyimide precursor is not particularly limited, and as an example, when a weight average molecular weight (in terms of polystyrene) of the polyimide precursor is 20,000 to 150,000 g/mol, a polyimide film having more excellent physical properties may be obtained.

According to an exemplary embodiment, the polyimide precursor composition may further contain additives such as a leveling agent, a flame retardant, an adhesion improver, an inorganic particle, an antioxidant, a UV stabilizer, and a plasticizer.

In addition, the present invention provides a method of producing a polyimide film, and specifically, the polyimide film according to the present invention may be produced by a method including: applying the polyimide precursor composition onto a substrate; and performing a heat treatment to form a polyimide film.

The polyimide is polyimide having a cyclic chemical structure (—CO—N—CO—) prepared by imidizing a polyimide precursor and dehydrating H of —CO—NH— and OH of —CO—OH in the polyimide precursor.

The imidization may be performed by a chemical imidization method or a thermal imidization method. For example, polyimide may be obtained by imidizing the polyimide precursor by a chemical reaction performed by adding a dehydrating agent and an imidization catalyst to the polymerized polyimide precursor composition and then heating the solution to a temperature of 50 to 120° C., or by imidizing the polyimide precursor by removing alcohol while refluxing the solution.

According to an exemplary embodiment, the imidization may be performed by a chemical imidization method.

In the chemical imidization method, as the imidization catalyst, pyridine, triethylamine, picoline, or quinoline may be used. In addition, a substituted or unsubstituted nitrogen-containing heterocyclic compound, an N-oxide compound of a nitrogen-containing heterocyclic compound, a substituted or unsubstituted amino acid compound, an aromatic hydrocarbon compound having a hydroxyl group, or an aromatic heterocyclic compound may be used. In particular, lower alkyl imidazoles such as 1,2-dimethylimidazole, N-methylimidazole, N-benzyl-2-methylimidazole, 2-methylimidazole, 2-ethyl-4-methylimidazole, and 5-methylbenzimidazole, substituted pyridines such as isoquinoline, 3,5-dimethylpyridine, 3,4-dimethylpyridine, 2,5-dimethylpyridine, 2,4-dimethylpyridine, and 4-n-propylpyridine, p-toluenesulfonic acid, and the like may be used. The imidization catalyst may be used in an amount of 0.1 to 5 mol with respect to 1 mol of the acid dianhydride.

As the dehydrating agent, an acid anhydride such as acetic anhydride, propionic anhydride, and benzoic anhydride, or acid chlorides thereof, a carbodiimide compound such as dicyclohexylcarbodiimide, and the like may be used. The dehydrating agent may be used in an amount of 0.1 to 10 mol with respect to 1 mol of the acid dianhydride.

During the chemical imidization, a heating process at a temperature of 50 to 120° C. may be performed together.

According to an exemplary embodiment, the imidization may be performed by a thermal imidization method. The imidization may be performed by applying the polyimide precursor composition onto a substrate and performing a heat treatment.

More specifically, the polyimide film according to the present invention may be produced by a production method including: preparing a polyimide-based polymer composition by imidizing the polyimide precursor composition; and applying the polyimide-based polymer composition onto a substrate and then performing a heat treatment (curing). Here, the imidization may be performed by applying the chemical imidization described above or a combination of the chemical imidization and the thermal imidization.

In addition, the polyimide film according to the present invention may be produced by a production method including applying the polyimide precursor composition onto a substrate and then performing a heat treatment (curing). Here, the imidization may be performed by applying the thermal imidization described above.

According to an exemplary embodiment, as the substrate, a glass substrate, a metal substrate, or a plastic substrate may be used without particular limitation. Among them, it is preferable to use a glass substrate or a metal substrate that has excellent thermal and chemical stability during processes of imidizing and curing the polyimide precursor composition, and may be easily separated without damage to the polyimide film formed after curing even without an additional treatment with a release agent.

Specifically, according to an exemplary embodiment, the application method is not particularly limited, and as an example, one or more methods selected from a spin coating method, a dipping method, a spray method, a die coating method, a bar coating method, a roll coating method, a meniscus coating method, a flexo printing method, a screen printing method, a bead coating method, an air knife coating method, a reverse roll coating method, a blade coating method, a casting coating method, and a gravure coating method may be used.

In an exemplary embodiment, the polyimide precursor composition or the polyimide-based polymer composition may be applied onto the substrate in a thickness range in which the final polyimide film may have an appropriate thickness suitable for a display device substrate. Specifically, the polyimide precursor composition or the polyimide-based polymer composition may be applied in an amount to have a thickness of 10 to 100 μm, but is not limited thereto, and the application amount may be adjusted as desired.

According to an exemplary embodiment, the heat treatment may be performed at 500° C. or lower, preferably, 80 to 500° C., and more preferably, 80 to 300° C.

According to an exemplary embodiment, the heat treatment may be performed in a total of 3 steps including: a first heat treatment step performed at 100° C. or lower, and specifically, 80 to 100° C.; a second heat treatment step performed at higher than 100° C. and 300° C. or lower; and a third heat treatment step performed at higher than 300° C. and 500° C. or lower, but the present invention is not limited thereto.

In an exemplary embodiment, the production method may further include, before the heat treatment (curing), drying the polyimide precursor composition to remove the organic solvent present in the polyimide precursor composition. The drying may be performed by a general method, and specifically, may be performed at 140° C. or lower, and preferably, 80° C. to 140° C.

In an exemplary embodiment, the production method may further include, after the heat treatment (curing), separating the polyimide film from the substrate.

In addition, the present invention provides a polyimide film produced using the polyimide precursor according to an exemplary embodiment.

In addition, the present invention provides a polyimide film containing the polyimide-based polymer composition according to an exemplary embodiment.

According to an exemplary embodiment, the polyimide film has a repeating unit represented by the following Chemical Formula 13:

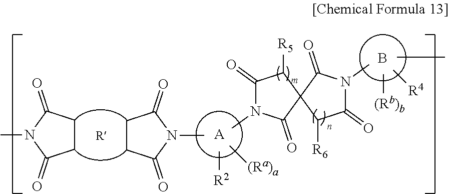

in Chemical Formula 13,

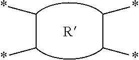

is at least one tetravalent group selected from

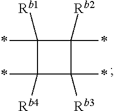

R^{a1}, R^{a2}, and R^{a3} are each independently C1-C10 alkyl or halo C1-C10 alkyl;

L is a single bond, C1-C10 alkylene, —O—, —S—, —CO—, —SO₂—, —SiR^{f1}R^{f2}—, —CO—Ar—CO—, or —O—Ar¹—(X—Ar²)ₛ—O—, and the alkylene may be further substituted with one or more selected from C1-C10 alkyl and halo C1-C10 alkyl;

R^{f1} and R^{f2} are each independently C1-C10 alkyl;

Ar, Ar¹, and Ar² are each independently C6-C20 arylene, and the arylene may be further substituted with one or more selected from C1-C10 alkyl and halo C1-C10 alkyl;

X is O or S;

R^{b1} to R^{b4} are each independently hydrogen, C1-C10 alkyl, or C6-C20 aryl;

a ring A and a ring B are each independently a C6-C20 aromatic ring or a C3-C20 heteroaromatic ring;

R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;

R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;

R^{b17}, R^{b18}, R^{c17}, and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

a and b are each independently an integer of 0 or 1;

m and n are each independently an integer of 1 to 5;

p, q, and r are each independently an integer of 0 to 2; and

s is an integer of 0 or 1.

According to an exemplary embodiment, in Chemical Formula 13,

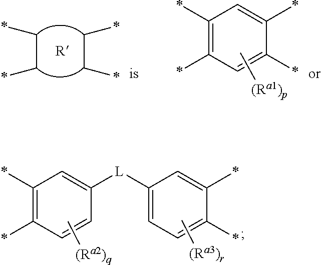

R^{a1}, R^{a2}, and R^{a3} are each independently C1-C10 alkyl or halo C1-C10 alkyl; L is a single bond, C1-C10 alkylene, —O—, —S—, —CO—, or —SO₂—, and the alkylene may be further substituted with one or more selected from C1-C10 alkyl and halo C1-C10 alkyl; and p, q, and r may be each independently an integer of 0 or 1.

According to an exemplary embodiment, the polyimide film may have a repeating unit represented by the following Chemical Formula 13-1:

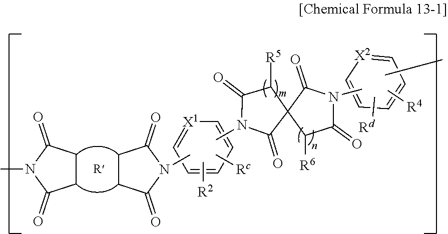

in Chemical Formula 13-1,

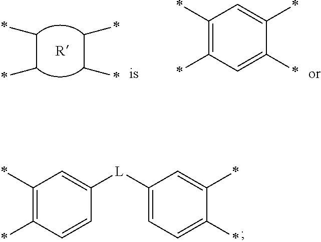

L is a single bond or —CR^{c1}R^{c2}—;

R^{c1} and R^{c2} are each independently C1-C10 alkyl or halo C1-C10 alkyl;

each of X¹ and X² is CR or N;

R², R⁴, R, R^{c}, and R^{d} are each independently hydrogen, halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano, and R² and R^{c}, and R⁴ and R^{d} may be linked to each other by C3-C5 alkylene or C3-C5 alkenylene to form a fused ring;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl; and

m and n are each independently an integer of 1 to 3.

More preferably, according to an exemplary embodiment, the polyimide film may have a repeating unit represented by the following Chemical Formula 13-2:

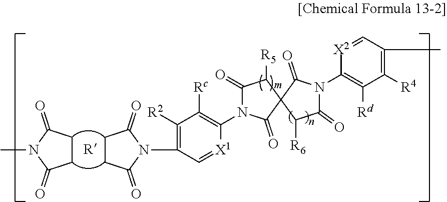

in Chemical Formula 13-2,

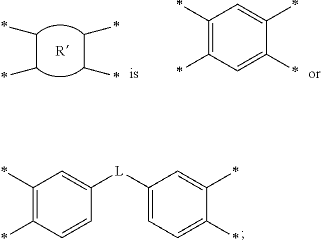

L is a single bond or —CR^{c1}R^{c2}—.

R^{c1} and R^{c2} are each independently C1-C6 alkyl or —C_{z}T¹_{2z+1};

each of X¹ and X² is CR or N;

R², R⁴, R, R^{c}, and R^{d} are each independently hydrogen, halogen, C1-C6 alkyl, —C_{z′}T²_{2z′+1}, hydroxy, C1-C6 alkoxy, —SiR^{e1}R^{e2}R^{e3}, or cyano, and R² and R^{c}, and R⁴ and R^{d} may be linked to each other by

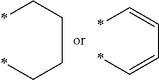

to form a fused ring;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or —C_{z″}T³_{2z″+1};

T¹, T², and T³ are each independently halogen;

z, z′, and z″ are each independently an integer of 1 to 6; and

m and n are each independently an integer of 1 or 2.

Specifically, according to an exemplary embodiment, in Chemical Formula 13-2,

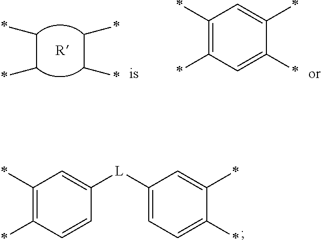

L is a single bond or —CR^{c1}R^{c2}—; R^{c1} and R^{c2} are each independently C1-C6 alkyl or —C_{z}T¹_{2z+1}; T¹ is halogen; and z may be an integer of 1 to 6, and more preferably, R^{c1} and R^{c2} may be each independently CH₃ or CF₃.

According to an exemplary embodiment, the polyimide film has a repeating unit represented by the following Chemical Formula 14:

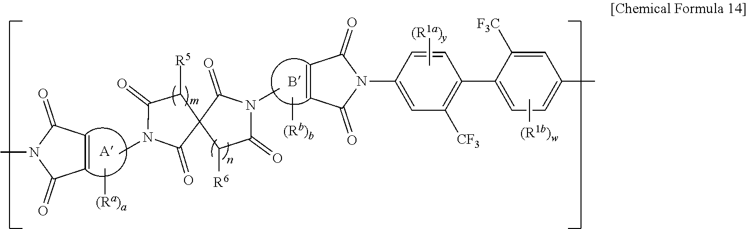

in Chemical Formula 14,

a ring A′ and a ring B′ are each independently a C6-C20 aromatic ring or a C3-C20 heteroaromatic ring;

R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;

R^{c11}, R^{c12}, R^{c13}, R^{c15}, and R^{c16} are each independently hydrogen, C1-C10 alkyl, C3-C10 cycloalkyl, or C6-C20 aryl;

R^{c17} and R^{c18} are each independently C1-C10 alkyl, C3-C10 cycloalkyl, or C6-C20 aryl;

R⁵ and R⁶ are each independently hydrogen, halogen, C1-C10 alkyl, or halo C1-C10 alkyl;

R^{1a} and R^{1b} are each independently halogen, C1-C10 alkyl, halo C1-C10 alkyl, C1-C10 alkoxy, or C6-C12 aryl;

a and b are each independently an integer of 0 or 1;

m and n are each independently an integer of 1 to 5; and

y and w are each independently an integer of 0 to 3.

According to an exemplary embodiment, the polyimide film may have a repeating unit represented by the following Chemical Formula 14-1:

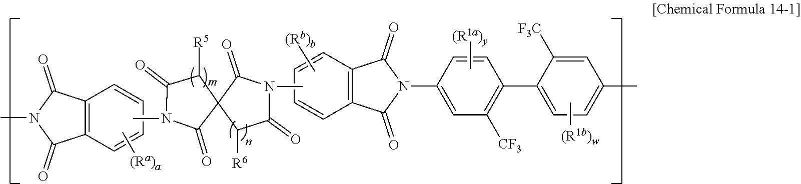

in Chemical Formula 14-1,

R^{a} and R^{b} are each independently halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano;

R^{e1} is hydrogen, C1-C10 alkyl, C3-C10 cycloalkyl, or C6-C20 aryl;

R^{e2} and R^{e3} are each independently C1-C10 alkyl, C3-C10 cycloalkyl, or C6-C20 aryl;

a and b are each independently an integer of 0 to 2;

R⁵ and R⁶ are each independently hydrogen, halogen, C1-C10 alkyl, or halo C1-C10 alkyl; and

m and n may be each independently an integer of 1 to 3.

According to an exemplary embodiment, the polyimide film may have a repeating unit represented by the following Chemical Formula 14-2:

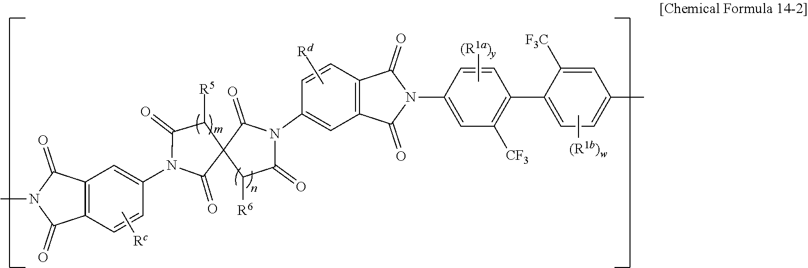

in Chemical Formula 14-2,

R^{1a} and R^{1b} are each independently halogen, C1-C6 alkyl, or halo C1-C6 alkyl;

R^{c} and R^{d} are each independently hydrogen, halogen, C1-C6 alkyl, halo C1-C6 alkyl, hydroxy, C1-C6 alkoxy, —SiR^{e1}R^{e2}R^{e3}, or cyano;

R^{e1} is hydrogen or C1-C6 alkyl;

R^{e2} and R^{e3} are each independently C1-C6 alkyl;

R⁵ and R⁶ are each independently hydrogen, halogen, C1-C6 alkyl, or halo C1-C6 alkyl;

m and n are each independently an integer of 1 or 2; and

y and w are each independently an integer of 0 or 1.

According to an exemplary embodiment, the polyimide film may further have a repeating unit represented by the following Chemical Formula G:

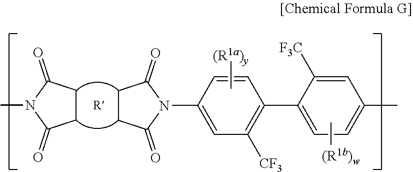

in Chemical Formula G,

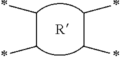

is the same as defined in Chemical Formula D;

R^{1a} and R^{1b} are each independently halogen, C1-C10 alkyl, halo C1-C10 alkyl, C1-C10 alkoxy, or C6-C12 aryl; and

y and w are each independently an integer of 0 to 3.

According to an exemplary embodiment, in Chemical Formula G,

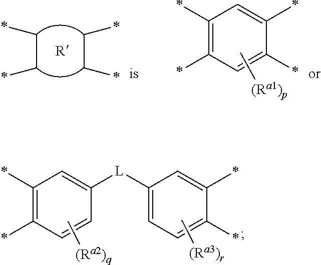

R^{a1}, R^{a2}, and R^{a3} are each independently C1-C6 alkyl or halo C1-C6 alkyl; L is a single bond, C1-C5 alkylene, —O—, —S—, —CO—, or —SO₂—, and the alkylene may be further substituted with one or more selected from C1-C6 alkyl and halo C1-C6 alkyl; R^{1a} and R^{1b} are each independently halogen, C1-C6 alkyl, or halo C1-C6 alkyl; y and w are each independently an integer of 0 or 1; and p, q, and r may be each independently an integer of 0 or 1.

Preferably, according to an exemplary embodiment, in Chemical Formula G,

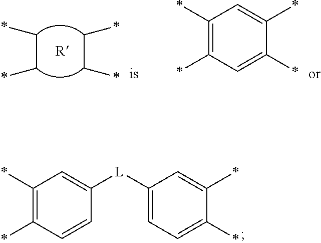

L is a single bond or —CR^{c1}R^{c2}—; R^{c1} and R^{c2} are each independently C1-C3 alkyl or halo C1-C3 alkyl, and more preferably, R^{c1} and R^{c2} may be each independently CH₃ or CF₃; R^{1a} and R^{1b} are each independently halogen, C1-C5 alkyl, or halo C1-C5 alkyl; and y and w are each independently an integer of 0 or 1, and more preferably, each of y and w may be 0.

According to an exemplary embodiment, the polyimide film essentially has a repeating unit represented by Chemical Formula 13 or 14, and may further have a repeating unit represented by Chemical Formula G. Preferably, according to an exemplary embodiment, the polyimide film essentially has a repeating unit represented by Chemical Formula 13-1 or 14-1, and may further have a repeating unit represented by Chemical Formula G. More preferably, according to an exemplary embodiment, the polyimide film essentially has a repeating unit represented by Chemical Formula 13-2 or 14-2, and may further have a repeating unit represented by Chemical Formula G.

According to an exemplary embodiment, the repeating unit represented by Chemical Formula 13 or 14 may be included in the polyimide in an amount of 10 to 100 mol %, preferably, 30 to 100 mol %, more preferably, 40 to 95 mol %, and still more preferably, 50 to 80 mol %.

According to an exemplary embodiment, the repeating unit represented by Chemical Formula G may be included in the polyimide in an amount of 90 mol % or less, 70 mol % or less, 5 to 60 mol %, or 20 to 50 mol % with respect to a total of mol % of the polyimide.

A weight average molecular weight (in terms of polystyrene) of the polyimide film according to an exemplary embodiment, that is, the polyimide, may be 10,000 to 200,000 g/mol, 20,000 to 100,000 g/mol, or 30,000 to 100,000 g/mol. In addition, a molecular weight distribution (Mw/Mn) of the polyimide according to the present invention may be in a range of 1.1 to 2.5. When the weight average molecular weight and the molecular weight distribution of the polyimide are in the above ranges, the polyimide film may have excellent properties such as optical properties, heat resistance, mechanical strength, and flexibility.

The polyimide film according to an exemplary embodiment is a colorless, transparent, and flexible polyimide film, and has a structural unit derived from a monomer having a specific structure with a central skeleton of diazaspiro tetrone, that is, the diazaspiro compound in the form of a diamine of Chemical Formula 1-1 or an acid dianhydride of Chemical Formula 1-2, such that the polyimide film has excellent optical properties such as a light transmittance, high heat resistance and mechanical strength, and further improved Young's modulus properties.

A Young's modulus of the polyimide film according to an exemplary embodiment may be 4.5 GPa or less, preferably, 4.0 GPa or less, more preferably, 3.5 to 4.5 GPa, and still more preferably, 3.5 to 4.0 GPa, when measured according to ASTM D882.

A yellow index (YI) of the polyimide film according to an exemplary embodiment may be 2.0 or less, preferably, 1.5 or less, more preferably, 1.0 to 2.0, and still more preferably, 1 to 1.5, when measured according to ASTM E313.

A haze of the polyimide film according to an exemplary embodiment may be 0.5% or less, specifically, 0.3% or less, and more specifically, 0.1 to 0.3%, when measured according to ASTM D1003.

A light transmittance of the polyimide film according to an exemplary embodiment may be 90% or more.

Specifically, the polyimide film may have a yellow index (YI) of 2.0 or less when measured according to ASTM E313, a light transmittance of 90% or more, a haze of 0.5% or less when measured according to ASTM D1003, and a Young's modulus of 4.0 GPa or less when measured according to ASTM D882.

More specifically, the polyimide film according to an exemplary embodiment may have a yellow index (YI) of 1.5 or less when measured according to ASTM E313, a light transmittance of 90% or more, a haze of 0.3% or less when measured according to ASTM D1003, and a Young's modulus of 4.0 GPa or less when measured according to ASTM D882.

Still more specifically, the polyimide film according to an exemplary embodiment may have a yellow index (YI) of 1.0 to 1.5 when measured according to ASTM E313, a light transmittance of 90% or more, a haze of 0.1 to 0.3% when measured according to ASTM D1003, and a Young's modulus of 3.5 to 4.0 GPa when measured according to ASTM D882.

In addition, the polyimide film according to the present invention may have excellent thermal stability according to a change in temperature.

That is, the polyimide film according to the present invention is produced using a monomer having a specific structure with a central skeleton of diazaspiro tetrone, that is, the diazaspiro compound in the form of a diamine of Chemical Formula 1-1 or an acid dianhydride of Chemical Formula 1-2, such that the polyimide film may have excellent optical properties and mechanical properties. Specifically, the polyimide film according to the present invention has a repeating unit derived from a monomer having a specific structure with a central skeleton of diazaspiro tetrone, that is, the diazaspiro compound in the form of a diamine of Chemical Formula 1-1 or an acid dianhydride of Chemical Formula 1-2, such that a highly transparent polyimide film having excellent optical properties, heat resistance, and mechanical strength, and in particular, having flexibility may be provided. Therefore, the polyimide film of the present invention may be used in various fields such as a device substrate, a display cover substrate, an optical film, an integrated circuit (IC) package, an adhesive film, a multi-layer flexible printed circuit (FPC), a tape, a touch panel, and an optical disk protective film.

The polyimide film according to an exemplary embodiment may be used as a laminate in which two or more layers are laminated.

In addition, the present invention provides a photoelectric device and a flexible display device that include the polyimide film or a laminate in which the polyimide films are laminated as a flexible substrate.

Examples of the photoelectric device include an optical component, a switch, and an optical modulator, and the photoelectric device is suitable for a high heat resistant substrate material requiring fine pattern formation characteristics.

Examples of the flexible display device include a liquid crystal display (LCD) device and an organic light emitting diode (OLED) device, and in particular, the flexible display device is suitable for an OLED device obtained by using a low temperature polysilicon (LTPS) process requiring a high-temperature process, but is not limited thereto.

Hereinafter, the present invention will be described with reference to specific Examples and Comparative Examples. The following Examples are illustrative only to describe the technical idea of the present invention, and those skilled in the art will appreciate that the present invention is not limited to Examples.

### [Example 1] Preparation of Diamine Monomer 1 (DA-1)

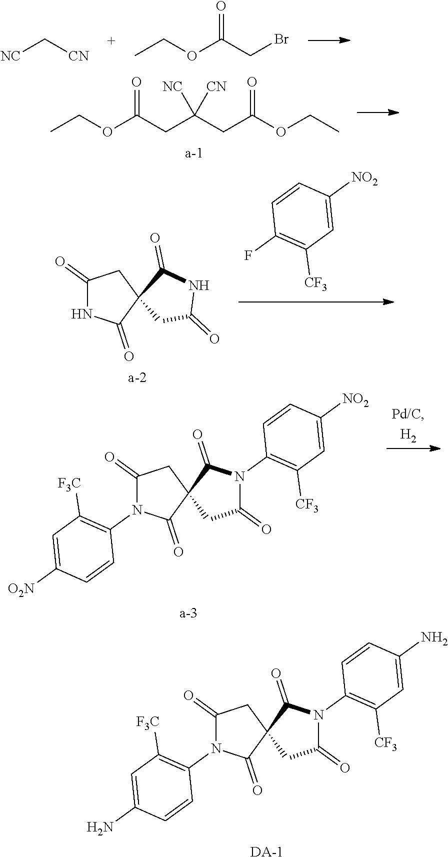

Step 1. Preparation of Compound a-1

To a 500 mL round-bottom flask, a stirrer bar, malononitrile (10 g), potassium carbonate (44 g), and dimethylformamide (100 mL) were added, and cooling was performed to 0° C. Ethyl bromoacetate (51 g) was added to the flask, the temperature was raised to room temperature, and stirring was performed for 2 hours. Dichloromethane (100 mL) and distilled water (100 mL) were added to separate layers, and then, an aqueous layer was removed. An organic layer was washed twice with distilled water (100 mL), the organic layer was dried with magnesium sulfate, and a solid was filtered. The filtrate was concentrated under reduced pressure until a solid was precipitated, hexane (100 mL) was added, stirring was performed for 1 hour, and filtration and drying were performed, thereby obtaining a compound a-1 (31 g).

¹H-NMR (500 MHz, DMSO) δ 4.28 (q, 4H), 3.21 (s, 4H), 1.33 (t, 6H)

Step 2. Preparation of Compound a-2

To a 250 mL round-bottom flask, a stirrer bar, the compound a-1 (31 g), sulfuric acid (7.8 mL), and acetic acid (77 mL) were added, and the temperature was raised to reflux. A solid was precipitated during the refluxing. After the refluxing was performed for 2 hours, cooling was performed to room temperature. The precipitated solid was filtered, the solid was washed three times with distilled water (30 mL), and the solid was dried, thereby obtaining a compound a-2 (18 g).

¹H-NMR (500 MHz, DMSO) δ 11.68 (br, 2H), 2.97-2.86 (m, 4H)

Step 3. Preparation of Compound a-3

To a 250 mL round-bottom flask, the compound a-2 (25 g), potassium carbonate (44 g), 2-fluoro-5-nitrobenzotrifluoride (60 g), and dimethylformamide (150 mL) were added, the temperature was raised to 40° C., and stirring was performed overnight. Thereafter, dilution was performed by adding ethyl acetate (400 mL), and washing was performed three times with distilled water (300 mL). An organic layer was concentrated under reduced pressure, methanol (700 mL) was added, refluxing was performed to precipitate a solid, and the precipitated solid was filtered and dried, thereby obtaining a compound a-3 (56 g).

¹H-NMR (500 MHz, DMSO) δ 8.81-8.58 (m, 4H), 8.10-7.74 (m, 2H), 3.79-3.27 (m, 4H)

Step 4. Preparation of Diamine Compound 1 (DA-1)

To a 1,000 mL autoclave, the compound a-3 (40 g), Pd/C (4 g), and methanol (800 g) were added. After the inside of the reactor was converted into a hydrogen atmosphere, hydrogen was charged up to 10 bar, and stirring was performed. After the stirring was performed overnight, the internal pressure was reduced to normal pressure, filtration was performed to remove Pd/C, and concentration was performed under reduced pressure. Isopropyl alcohol (400 mL) was added to the concentrated residue, the temperature was raised to reflux for 1 hour, and then, cooling was performed to room temperature. The generated solid was filtered and dried to obtain a diamine compound 1 (DA-1) (24 g).

¹H-NMR (500 MHz, DMSO) δ 7.19-6.81 (m, 6H), 6.97 (dd, 4H), 3.54-3.09 (m, 4H)

### [Example 2] Preparation of Acid Dianhydride Monomer 1 (ADA-1)

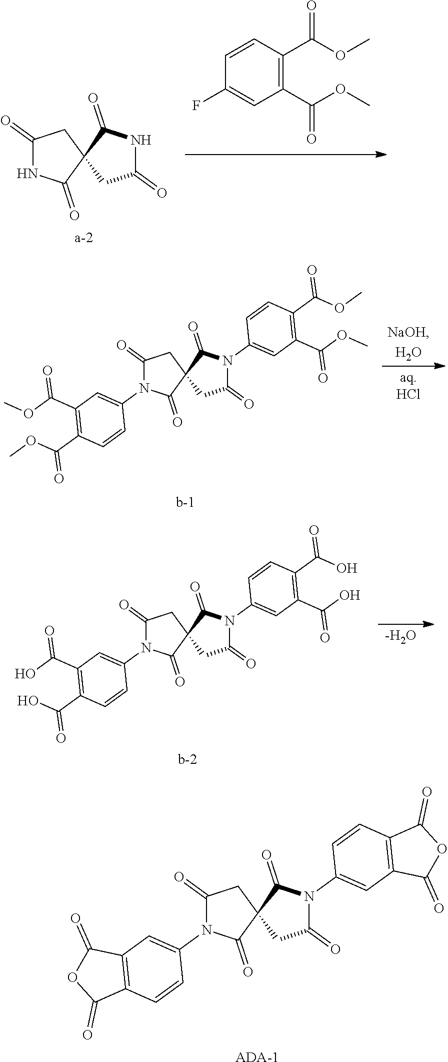

Step 1. Preparation of Compound b-1

A compound b-1 (35 g) was obtained in the same manner as in Step 3 of Example 1, except that dimethyl-4-fluorophthalate (60 g) was used instead of 2-fluoro-5-nitrobenzotrifluoride.

¹H-NMR (500 MHz, DMSO) δ 7.67 (s, 2H), 7.16-7.09 (m, 4H), 3.13-2.88 (m, 4H), 2.22 (s, 12H)

Step 2. Preparation of Compound b-2

To a 1,000 mL round-bottom flask, a stirrer bar, the compound b-1 (35 g), sodium hydroxide (20 g), and distilled water (700 mL) were added, and stirring was performed at room temperature overnight until the compounds were completely dissolved. A 35% hydrochloric acid aqueous solution was added until a pH was 6 to 7, and concentration was performed under reduced pressure until a solid was precipitated to remove distilled water. The generated solid was filtered and dried to obtain a compound b-2 (20 g).

¹H-NMR (500 MHz, DMSO) δ 13.30 (br, 4H), 8.59-8.50 (m, 4H), 7.74 (s, 2H), 3.15-2.30 (m, 4H)

Step 3. Preparation of Acid Dianhydride Compound 1 (ADA-1)

To a 250 mL round-bottom flask, a stirrer bar, the compound b-2 (20 g), and acetic anhydride (200 mL) were added, the temperature was raised to reflux overnight, and then, cooling was performed to room temperature. The generated solid was filtered, washing was performed twice with dichloromethane (20 mL), and then, drying was performed, thereby obtaining an acid dianhydride compound 1 (ADA-1) (15 g).

¹H-NMR (500 MHz, DMSO) δ 8.56 (s, 2H), 8.48 (d, 2H), 7.72 (d, 2H), 3.15-2.86 (m, 4H)

### [Example 3] Preparation of Diamine Monomer 2 (DA-2)

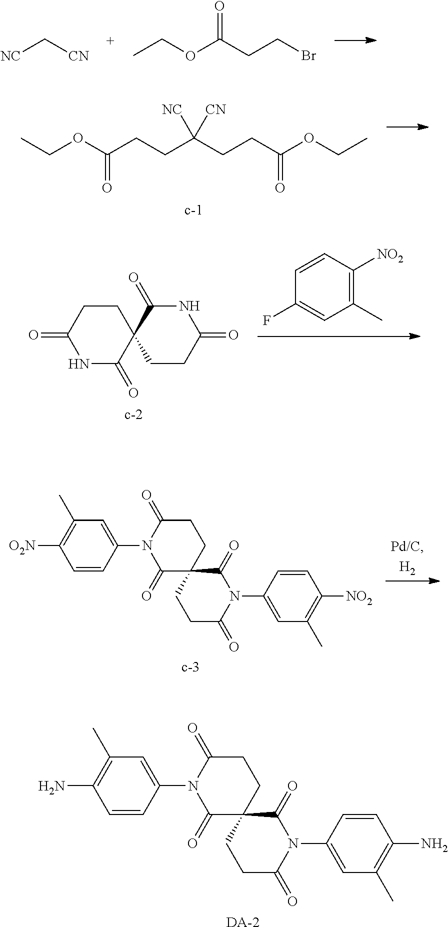

Step 1. Preparation of Compound c-1

A compound c-1 (30 g) was obtained in the same manner as in Step 1 of Example 1, except that ethyl 3-bromopropionate (60 g) was used instead of ethyl bromoacetate.

¹H-NMR (500 MHz, DMSO) δ 4.15 (q, 4H), 2.35-2.30 (m, 8H), 1.33 (t, 6H)

Step 2. Preparation of Compound c-2

A compound c-2 (15 g) was obtained in the same manner as in Step 2 of Example 1, except that the compound c-1 (30 g) was used instead of the compound a-1.

¹H-NMR (500 MHz, DMSO) δ 11.70 (br, 2H), 2.30-2.00 (m, 8H)

Step 3. Preparation of Compound c-3

A compound c-3 (45 g) was obtained in the same manner as in Step 3 of Example 1, except that the compound c-2 (25 g) was used instead of the compound a-2 and 5-fluoro-2-nitrotoluene (55 g) was used instead of 2-fluoro-5-nitrobenzotrifluoride.

¹H-NMR (500 MHz, DMSO) δ 8.24-8.20 (m, 2H), 7.98 (s, 2H), 7.75-7.70 (m, 2H), 2.55 (s, 6H), 2.46-2.16 (m, 8H)

Step 4. Preparation of Diamine Compound 2 (DA-2)

A diamine compound 2 (DA-2) (44 g) was obtained in the same manner as in Step 4 of Example 1, except that the compound c-3 (40 g) was used instead of the compound a-3.

¹H-NMR (500 MHz, DMSO) δ 7.50 (s, 2H), 7.03-6.95 (m, 6H), 6.85 (d, 2H), 2.45-2.20 (m, 8H), 2.12 (s, 6H)

### [Example 4] Preparation of Diamine Monomer 3 (DA-3)

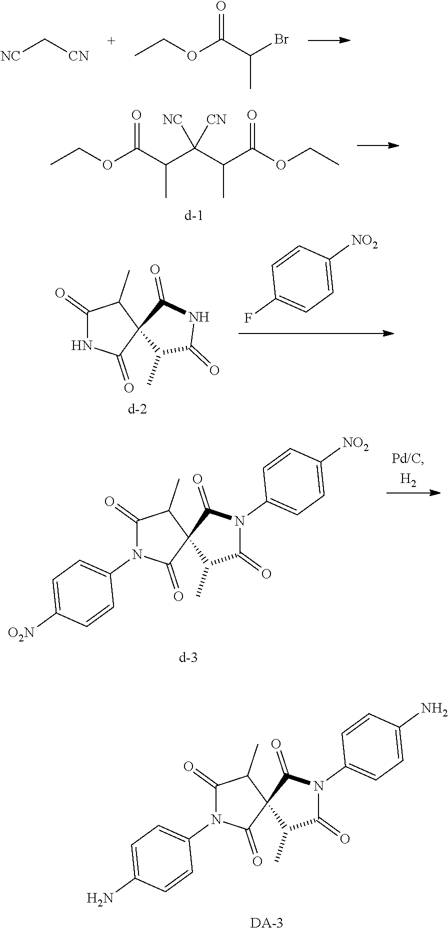

Step 1. Preparation of Compound d-1

A compound d-1 (31 g) was obtained in the same manner as in Step 1 of Example 1, except that ethyl 2-bromopropionate (60 g) was used instead of ethyl bromoacetate.

¹H-NMR (500 MHz, DMSO) δ 4.21 (q, 4H), 2.30 (q, 2H), 1.21 (t, 6H), 1.12 (d, 6H)

Step 2. Preparation of Compound d-2

A compound d-2 (16 g) was obtained in the same manner as in Step 2 of Example 1, except that the compound d-1 (30 g) was used instead of the compound a-1.

¹H-NMR (500 MHz, DMSO) δ 11.70 (br, 2H), 3.03 (q, 2H), 1.13 (d, 6H)

Step 3. Preparation of Compound d-3

A compound d-3 (40 g) was obtained in the same manner as in Step 3 of Example 1, except that the compound d-2 (25 g) was used instead of the compound a-2 and 1-fluoro-4-nitrobenzene (55 g) was used instead of 2-fluoro-5-nitrobenzotrifluoride.

¹H-NMR (500 MHz, DMSO) δ 8.40 (d, 4H), 7.80 (d, 4H), 3.03 (q, 6H), 1.12 (s, 6H)

Step 4. Preparation of Diamine Compound 3 (DA-3)

A diamine compound 3 (DA-3) (32 g) was obtained in the same manner as in Step 4 of Example 1, except that the compound d-3 (40 g) was used instead of the compound a-3.

¹H-NMR (500 MHz, DMSO) δ 7.12 (d, 4H), 6.22 (d, 4H), 4.52 (br, 4H), 3.00 (q, 2H), 1.11 (s, 6H)

### [Example 5] Preparation of Diamine Monomer 4 (DA-4)

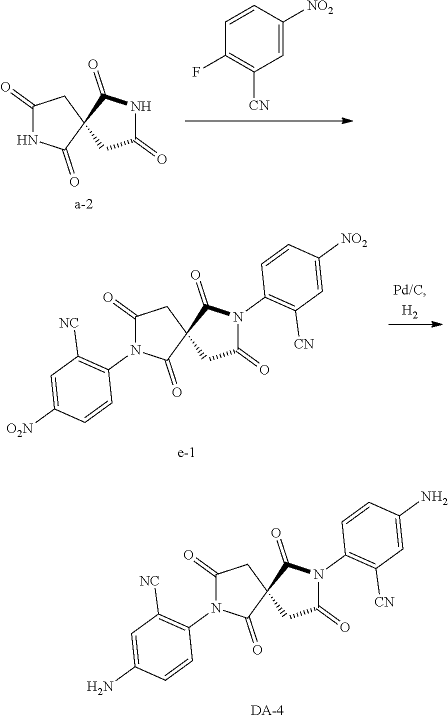

Step 1. Preparation of Compound e-1

A compound e-1 (30 g) was obtained in the same manner as in Step 3 of Example 1, except that 2-fluoro-5-nitrobenzonitrile (58 g) was used instead of 2-fluoro-5-nitrobenzotrifluoride.

¹H-NMR (500 MHz, DMSO) δ 8.54-8.45 (m, 4H), 8.09 (d, 2H), 3.13-2.88 (m, 4H)

Step 2. Preparation of Diamine Compound 4 (DA-4)

A diamine compound 4 (DA-4) (35 g) was obtained in the same manner as in Step 4 of Example 1, except that the compound e-1 (40 g) was used instead of the compound a-3.

¹H-NMR (500 MHz, DMSO) δ 7.68 (d, 2H), 7.15 (s, 2H), 6.50 (d, 2H), 4.78 (br, 4H), 3.13-2.88 (m, 4H)

### [Example 6] Preparation of Polyimide Precursor Composition

6FDA/Diamine Compound 1 (Molar Ratio: 1/0.99)

After a reactor through which a nitrogen stream flowed was filled with N,N-dimethylacetamide (DMAc) (67.89 mL), the diamine compound 1 (22.0 mmol) prepared in Example 1 was added while the temperature of the reactor was maintained at 25° C., and stirring was sufficiently performed. After confirming that the compound was completely dissolved, 4,4′-(hexafluoroisopropylidene)diphthalic anhydride (6FDA) (22.22 mmol) was added at the same temperature, stirring was sufficiently performed, and polymerization was performed at 40° C. for 18 hours. At this time, the solid content was 24.71 wt %. Subsequently, pyridine (55.55 mmol) and acetic anhydride (55.55 mmol) that were used as a catalyst and a dehydrating agent, respectively, were sequentially added to the solution, and stirring was performed at 60° C. for 24 hours, thereby preparing a polyimide-based polymer solution. The viscosity of the prepared polyimide-based polymer solution was 16,470 cPs.

### [Example 7] Preparation of Polyimide Precursor Composition

Acid Dianhydride Compound 1/TFMB (Molar Ratio: 1/0.99)

A polyimide precursor solution was prepared in the same manner as that of Example 6, except that the acid dianhydride compound 1 was used instead of 6FDA and 2,2′-bis(trifluoromethyl)-benzidine (TFMB) was used instead of the diamine compound 1. The viscosity of the polyimide precursor solution was 14,520 cPs.

### [Example 8] Preparation of Polyimide Precursor Composition

6FDA/Diamine Compound 2 (Molar Ratio: 1/0.99)

A polyimide precursor solution was prepared in the same manner as that of Example 6, except that the diamine compound 2 was used instead of the diamine compound 1. The viscosity of the polyimide precursor solution was 15,690 cPs.

### [Example 9] Preparation of Polyimide Precursor Composition

6FDA/Diamine Compound 3 (Molar Ratio: 1/0.99)

A polyimide precursor solution was prepared in the same manner as that of Example 6, except that the diamine compound 3 was used instead of the diamine compound 1. The viscosity of the polyimide precursor solution was 13,980 cPs.

### [Example 10] Preparation of Polyimide Precursor Composition

6FDA/Diamine Compound 4 (Molar Ratio: 1/0.99)

A polyimide precursor solution was prepared in the same manner as that of Example 6, except that the diamine compound 4 was used instead of the diamine compound 1. The viscosity of the polyimide precursor solution was 16,130 cPs.

### [Comparative Example 1] Production of Polyimide Precursor Composition

6FDA/TFMB (Molar Ratio: 1/0.99)

After a reactor through which a nitrogen stream flowed was filled with N,N-dimethylacetamide (DMAc) (67.89 mL), 2,2′-bis(trifluoromethyl)-benzidine (TFMB) was added while the temperature of the reactor was maintained at 25° C., and stirring was sufficiently performed. After confirming that the compound was completely dissolved, 6FDA (22.22 mmol) was added at the same temperature, stirring was sufficiently performed, and polymerization was performed at 40° C. for 18 hours. At this time, the solid content was 16.61 wt %. Subsequently, pyridine (55.55 mmol) and acetic anhydride (55.55 mmol) that were used as a catalyst and a dehydrating agent, respectively, were sequentially added to the solution, and stirring was performed at 60° C. for 24 hours, thereby preparing a polyimide-based polymer solution. The viscosity of the polyimide precursor solution was 15,480 cPs.

### [Experimental Example 1] Production of Polyimide Film

After the polyimide-based polymer solution was thinly spread on a glass plate, drying was performed at 90° C. for 20 minutes. Subsequently, the dried film was separated from the support of the glass plate, the substrate film was fixed using a pin tenter, and the substrate film was subjected to a heat treatment for 1 hour while the temperature was raised to a maximum of 280° C. at a temperature rise rate of 10° C./min, thereby producing a polyimide film.

The physical properties of the polyimide film produced by the above method were evaluated by the following methods. The results are shown in Table 1.

[Evaluation Methods]

1) Film thickness: After 0.5 T glass was coated with PAA and curing was performed, a thickness of the cured substrate was measured using a film thickness gauge (Alpha step D500, KLA Corporation). A unit of the thickness is μm.

2) Total light transmittance: A total light transmittance of a polyimide film having a thickness of 10 μm was measured in the entire wavelength range of 380 to 780 nm according to the ASTM D1746 standard using a spectrophotometer (MPC-3100, Shimadzu Corporation). A unit of the total light transmittance is %.

3) Haze: A haze of a polyimide film having a thickness of 10 μm was measured according to the ASTM D1003 standard using a haze meter HM-150. A unit of the haze is %.

4) Yellow index (YI): A yellow index of a polyimide film having a thickness of 10 μm was measured according to the ASTM E313 standard using a colorimeter (ColorQuest XE, Hunter Associates Laboratory, Inc.).

5) Young's modulus: A Young's modulus of a polyimide film having a thickness of 10 μm, a length of 40 mm, and a width of 5 mm was measured under a condition in which the polyimide film was pulled at 25° C. and 10 mm/min according to ASTM D882 using a UTM 3365 (Instron Corporation). A unit of the modulus is GPa.

TABLE 1

| | | | | | | Comparative |
|---|---|---|---|---|---|---|
| | Example 6 | Example 7 | Example 8 | Example 9 | Example 10 | Example 1 |
| Diamine | Diamine | TFMB | Diamine | Diamine | Diamine | TFMB |
| monomer | compound 1 | | compound 2 | compound 3 | compound 4 | |
| | (Example 1) | | (Example 3) | (Example4) | (Example5) | |
| Acid | 6FDA | Acid | 6FDA | 6FDA | 6FDA | 6FDA |
| dianhydride | | dianhydride | | | | |
| monomer | | compound 1 | | | | |
| | | (Example 2) | | | | |
| Thickness (μm) | 39 | 38 | 39 | 38 | 38 | 42 |
| Light | 92.1 | 92.1 | 92.3 | 92.0 | 92.1 | 92.1 |
| transmittance | | | | | | |
| (%) | | | | | | |
| Haze (%) | 0.12 | 0.16 | 0.15 | 0.13 | 0.12 | 0.39 |
| YI | 1.5 | 1.5 | 1.4 | 1.5 | 1.5 | 1.6 |
| Modulus (GPa) | 3.8 | 3.9 | 3.9 | 3.8 | 3.8 | 4.8 |

As shown in Table 1, it can be confirmed that in the cases of the polyimide films obtained in Examples 6 to 10, the yellow index (YI) is low, the haze is low, and the transmittance to visible light is high, such that the optical properties are excellent, and the physical properties are superior to those in Comparative Example 1 in terms of the Young's modulus.

That is, it can be confirmed that each of the polyimide films of Examples 6 to 10 has the structural unit derived from a monomer having a specific structure with a central skeleton of diazaspiro tetrone according to the present invention, such that the residual stress is reduced and further improved flexibility is exhibited.

Therefore, a colorless and transparent polyimide film having excellent flexibility, excellent optical properties, and improved Young's modulus properties may be produced using the monomer having a specific structure with a central skeleton of diazaspiro tetrone according to the present invention.

As set forth above, the diazaspiro compound having a structure according to the present invention is a spiro compound of an azacyclic dione containing one nitrogen atom and carbon atoms of two carbonyl groups as ring atoms, in which an aromatic ring or a heteroaromatic ring is substituted for the nitrogen atom, and the aromatic ring or the heteroaromatic ring is substituted with a specific substituent. That is, the diazaspiro compound according to the present invention is a compound having a structure with a central skeleton of diazaspiro tetrone.

In particular, in a case where an amino group (—NH₂) is substituted for the aromatic ring or the heteroaromatic ring to be substituted for the nitrogen atom of the central skeleton of diazaspiro tetrone, the diazaspiro compound may be significantly useful as a diamine monomer for synthesizing a polyimide-based polymer. In addition, in a case where an acid anhydride group (—C(═O)—O—C(═O)—) is fused at the aromatic ring or the heteroaromatic ring, the diazaspiro compound may be significantly useful as an acid dianhydride monomer for synthesizing a polyimide-based polymer.

According to the present invention, a monomer for synthesizing polyimide that may implement excellent optical properties and improved Young's modulus properties may be provided, and a highly transparent and flexible polyimide film having improved physical properties, particularly, improved Young's modulus properties may be produced using the same.

That is, polyimide has a structural unit derived from the diazaspiro compound having a structure according to the present invention, such that Young's modulus properties may be significantly improved, thereby increasing flexibility of the polyimide.

That is, the polyimide film according to the present invention has a structural unit derived from the diazaspiro compound having a structure, such that the polyimide film may have excellent optical properties such as a yellow index, a light transmittance, and a haze and may also have further improved flexibility due to improved Young's modulus properties.

Therefore, the polyimide film according to the present invention has excellent transparency and heat resistance and improved flexibility, and thus may be significantly advantageously used in various fields such as a device substrate, a flexible display device substrate, an optical film, an integrated circuit (IC) package, an adhesive film, a multi-layer flexible printed circuit (FPC), a tape, a touch panel, and an optical disk protective film.

Hereinabove, although the present invention has been described by specific matters and limited exemplary embodiments, they have been provided only for assisting in the entire understanding of the present invention. Therefore, the present invention is not limited to the exemplary embodiments. Various modifications and changes may be made by those skilled in the art to which the present invention pertains from this description. Therefore, the spirit of the present invention should not be limited to the described exemplary embodiments, but the claims and all modifications equal or equivalent to the claims are intended to fall within the spirit of the present invention.

What is claimed is:

1. A diazaspiro compound represented by the following Chemical Formula 1: 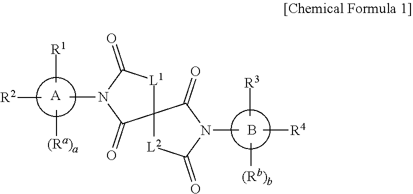
in Chemical Formula 1,
a ring A and a ring B are each independently a C6-C20 aromatic ring or a C3-C20 heteroaromatic ring;
L¹ and L² are each independently substituted or unsubstituted hydrocarbylene;
R¹ and R³ are each independently halogen, substituted or unsubstituted hydrocarbyl, —OR^{a11}, —NR^{a12}R^{a13}, —COR^{a14}, —COOR^{a15}, nitro, —SiR^{a16}R^{a17}R^{a18}, or cyano;
R² and R⁴ are each independently hydrogen, halogen, substituted or unsubstituted hydrocarbyl, —OR^{b11}, —NR^{b12}R^{b13}, —COR^{b14}, —COOR^{b15}, nitro, —SiR^{b16}R^{b17}R^{b18}, or cyano;
R¹ and R² may be linked to each other to form a fused ring, and R³ and R⁴ may be linked to each other to form a fused ring;
R^{a} and R^{b} are each independently halogen, substituted or unsubstituted hydrocarbyl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring;
R^{a11} to R^{a18}, R^{b11} to R^{b18}, and R^{c11} to R^{c18} are each independently hydrogen or substituted or unsubstituted hydrocarbyl; and
a and b are each independently an integer of 0 to 2, when a is an integer of 2, each of the R^{a}(s) may be the same as or different from each other, and when b is an integer of 2, each of the R^{b}(s) may be the same as or different from each other.

2. The diazaspiro compound of claim 1, wherein the substituted hydrocarbylene and the substituted hydrocarbyl are hydrocarbylene and hydrocarbyl substituted with one or more selected from the group consisting of halogen, hydroxy, C1-C10 alkyl, halo C1-C10 alkyl, C1-C10 alkoxy, —NR′R″ (where R′ and R″ are each independently hydrogen, C1-C10 alkyl, or C6-C20 aryl), nitro, and cyano.

3. The diazaspiro compound of claim 1, wherein the diazaspiro compound is represented by the following Chemical Formula 2: 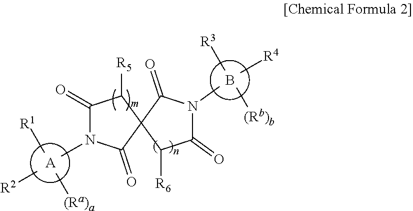
in Chemical Formula 2,
a ring A, a ring B, R¹, R², R³, R⁴, R^{a}, R^{b}, a, and b are the same as defined in Chemical Formula 1 of claim 1;
R⁵ and R⁶ are each independently hydrogen, halogen, C1-C10 alkyl, or halo C1-C10 alkyl; and
m and n are each independently an integer of 1 to 5.

4. The diazaspiro compound of claim 3, wherein R¹ and R³ are each independently halogen, C1-C10 alkyl, halo C1-C10 alkyl, C3-C10 cycloalkyl, C6-C20 aryl, —OR^{a11}, —NR^{a12}R^{a13}, —COOR^{a15}, nitro, —SiR^{a16}R^{a17}R^{a18}, or cyano;
R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;
R¹ and R² may be linked to each other by 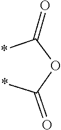 [SMILES: *C(=O)OC(*)=O]
to form a fused ring, and R³ and R⁴ may be linked to each other by 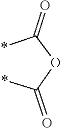 [SMILES: *C(=O)OC(*)=O]
to form a fused ring;
R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;
R^{a11}, R^{a12}, R^{a13}, R^{a15}, R^{a16}, R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;
R^{a17}, R^{a18}, R^{b17}, R^{b18}, R^{c17}, and R^{c18} are each independently C1-C10 alkyl, C3-C10 cycloalkyl, or C6-C20 aryl; and
a and b are each independently an integer of 0 or 1.

5. The diazaspiro compound of claim 4, wherein the diazaspiro compound is represented by the following Chemical Formula 3: 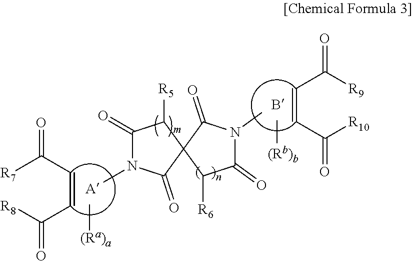
in Chemical Formula 3, 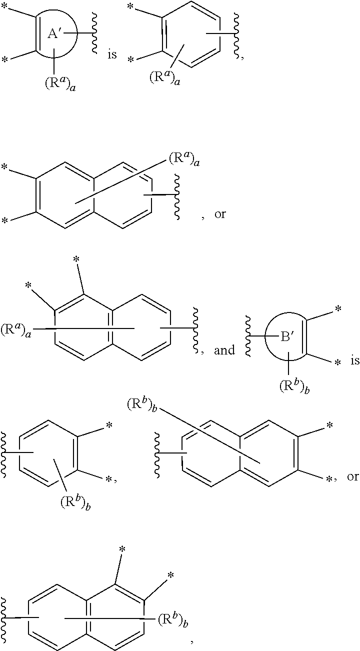
where a wavy line [symbol] represents a bonding site to a nitrogen atom, and an asterisk (*) represents a bonding site to a carbon atom;
R⁵ and R⁶ are each independently hydrogen, halogen, C1-C10 alkyl, or halo C1-C10 alkyl;
R⁷ to R¹⁰ are each independently hydroxy, C1-C10 alkoxy, C3-C10 cycloalkyloxy, or C6-C20 aryloxy, and R⁷ and R⁸, and R⁹ and R¹⁰ may be linked to each other by *—O—* to form a fused ring;
R^{a} and R^{b} are each independently halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano;
R^{e1} is hydrogen, C1-C10 alkyl, C3-C10 cycloalkyl, or C6-C20 aryl;
R^{e2} and R^{e3} are each independently C1-C10 alkyl, C3-C10 cycloalkyl, or C6-C20 aryl;
a and b are each independently an integer of 0 to 2; and
m and n are each independently an integer of 1 to 3.

6. The diazaspiro compound of claim 5, wherein the diazaspiro compound is represented by the following Chemical Formula 3-1, 3-2, or 3-3: 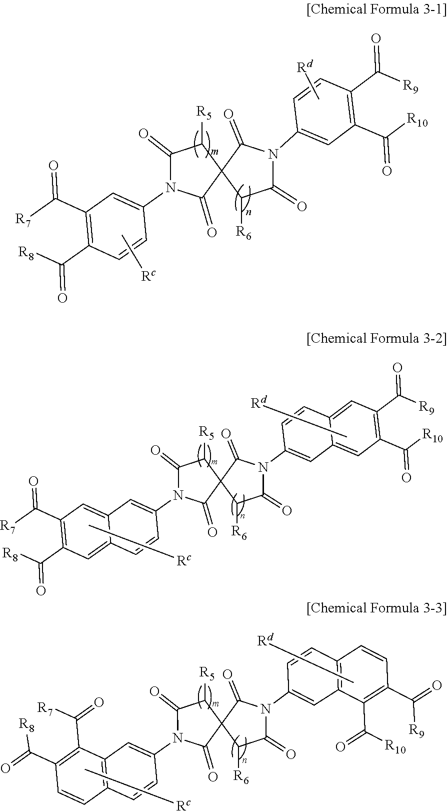
in Chemical Formula 3-1, 3-2, or 3-3,
R^{c} and R^{d} are each independently hydrogen, halogen, C1-C6 alkyl, halo C1-C6 alkyl, hydroxy, C1-C6 alkoxy, —SiR^{e1}R^{e2}R^{e3}, or cyano;
R^{e1} is hydrogen or C1-C6 alkyl;
R^{e2} and R^{e3} are each independently C1-C6 alkyl;
R⁵ and R⁶ are each independently hydrogen, halogen, C1-C6 alkyl, or halo C1-C6 alkyl;
R⁷ to R¹⁰ are each independently hydroxy or C1-C6 alkoxy, and R⁷ and R⁸, and R⁹ and R¹⁰ may be linked to each other by *—O—* to form a fused ring; and
m and n are each independently an integer of 1 or 2.

7. The diazaspiro compound of claim 6, wherein the diazaspiro compound is selected from the following compounds:

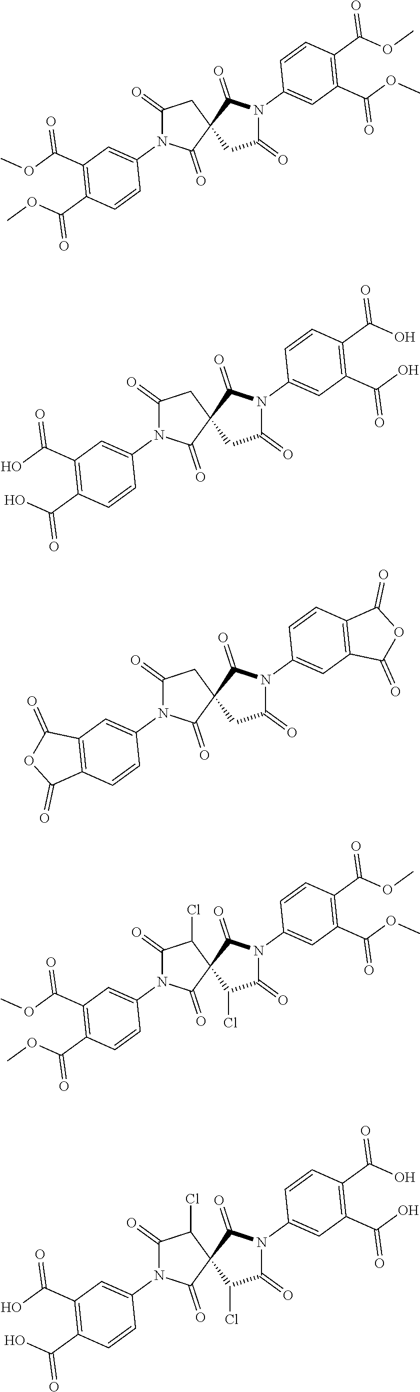

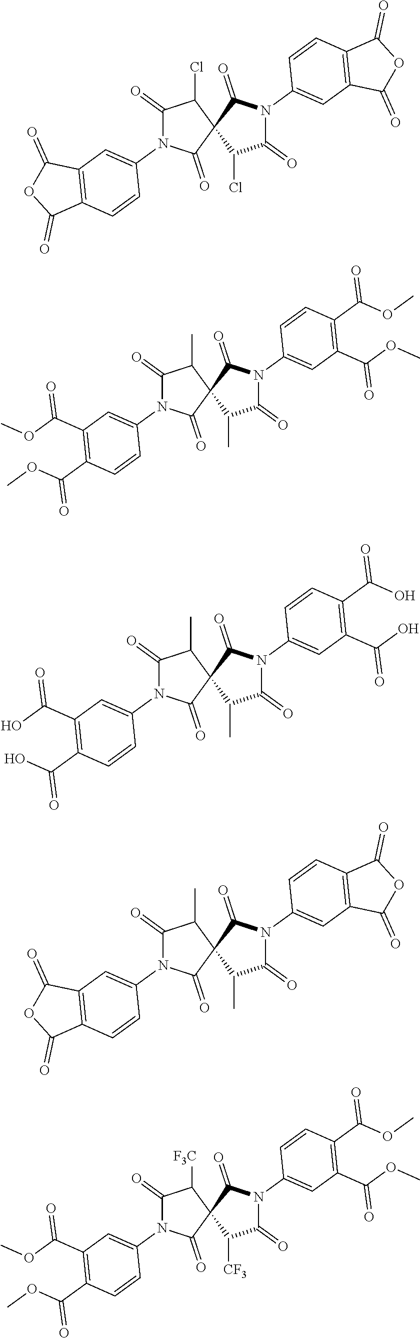

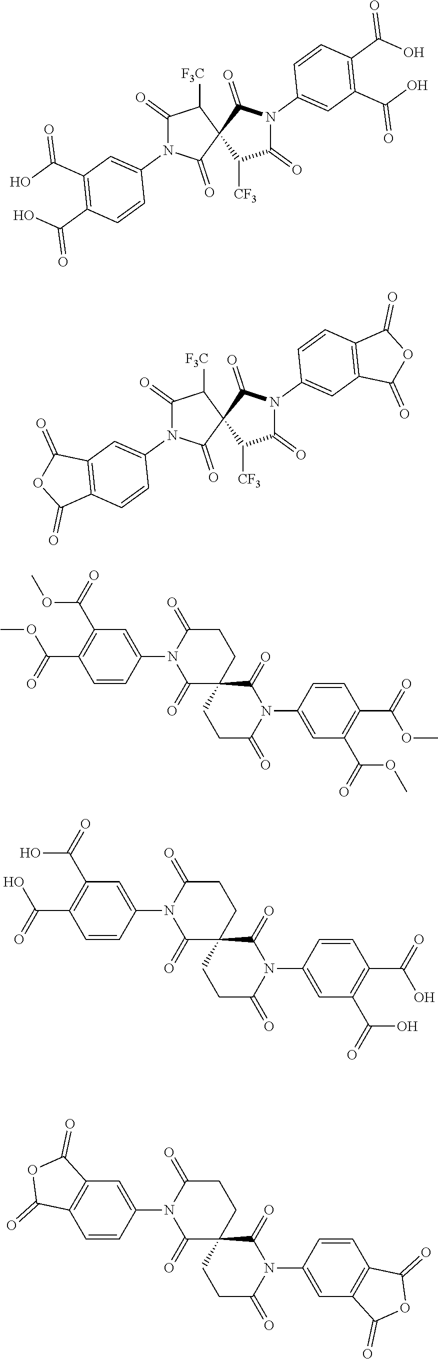

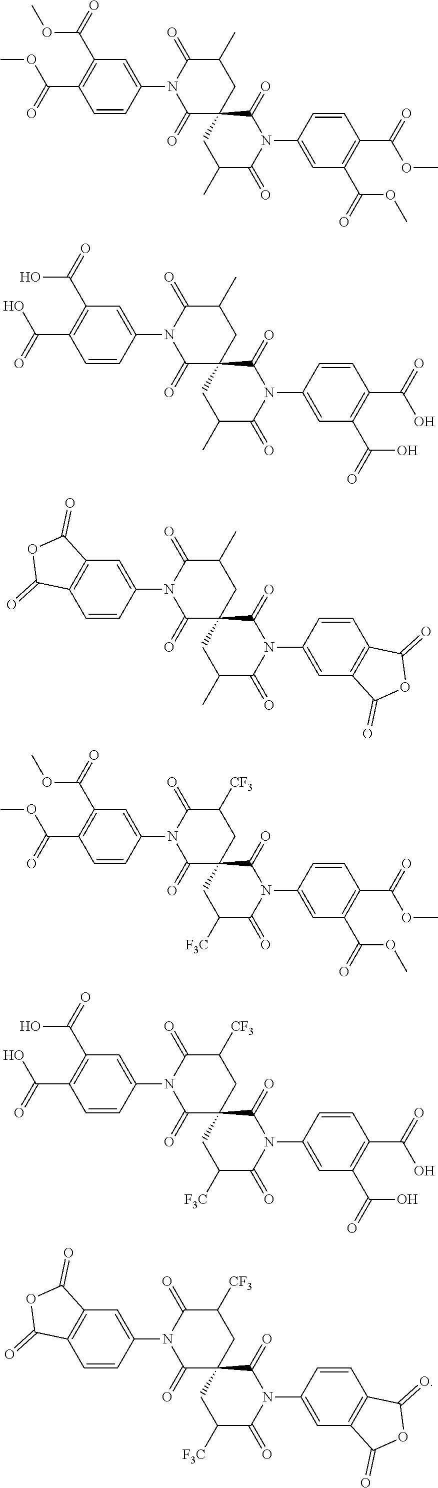

8. The diazaspiro compound of claim 4, wherein the diazaspiro compound is represented by the following Chemical Formula 4: 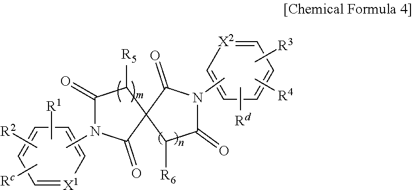
in Chemical Formula 4,
each of X¹ and X² is CR or N;
R¹ and R³ are each independently —NH₂ or nitro;
R², R⁴, R, R^{c}, and R^{d} are each independently hydrogen, halogen, C1-C10 alkyl, halo C1-C10 alkyl, C3-C10 cycloalkyl, C6-C20 aryl, hydroxy, C1-C10 alkoxy, C3-C10 cycloalkyloxy, C6-C20 aryloxy, —SiR^{e1}R^{e2}R^{e3}, or cyano, and R² and R^{c}, and R⁴ and R^{d} may be linked to each other by C3-C5 alkylene or C3-C5 alkenylene to form a fused ring;
R^{e1} is hydrogen, C1-C10 alkyl, C3-C10 cycloalkyl, or C6-C20 aryl;
R^{e2} and R^{e3} are each independently C1-C10 alkyl, C3-C10 cycloalkyl, or C6-C20 aryl;
R⁵ and R⁶ are each independently hydrogen, halogen, C1-C10 alkyl, or halo C1-C10 alkyl; and
m and n are each independently an integer of 1 to 3.

9. The diazaspiro compound of claim 8, wherein the diazaspiro compound is represented by the following Chemical Formula 4-1: 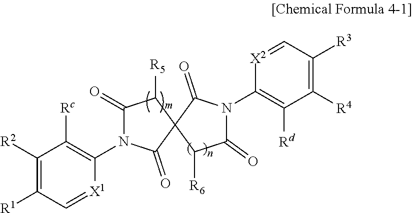
in Chemical Formula 4-1,
each of X¹ and X² is CR or N;
R¹ and R³ are each independently —NH₂ or nitro;
R², R⁴, R, R^{c}, and R^{d} are each independently hydrogen, halogen, C1-C6 alkyl, halo C1-C6 alkyl, hydroxy, C1-C6 alkoxy, —SiR^{e1}R^{e2}R^{e3}, or cyano, and R² and R^{c}, and R⁴ and R^{d} may be linked to each other by 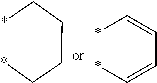
to form a fused ring;
R^{e1} is hydrogen or C1-C6 alkyl;
R^{e2} and R^{e3} are each independently C1-C6 alkyl;
R⁵ and R⁶ are each independently hydrogen, halogen, C1-C6 alkyl, or halo C1-C6 alkyl; and
m and n are each independently an integer of 1 or 2.

10. The diazaspiro compound of claim 9, wherein the diazaspiro compound is selected from the following compounds:

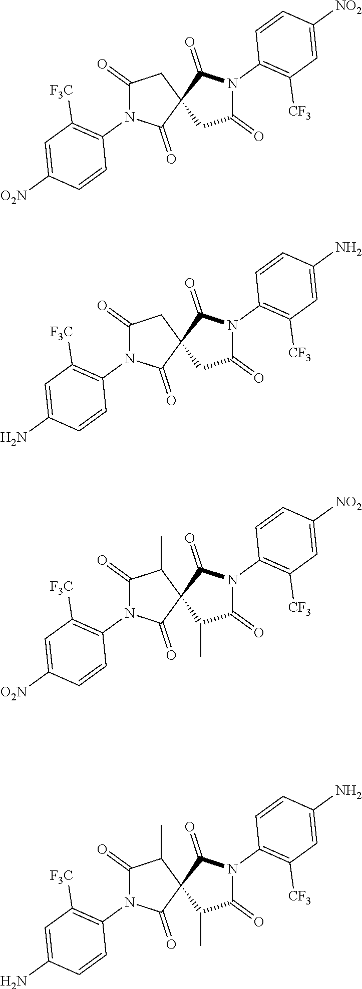

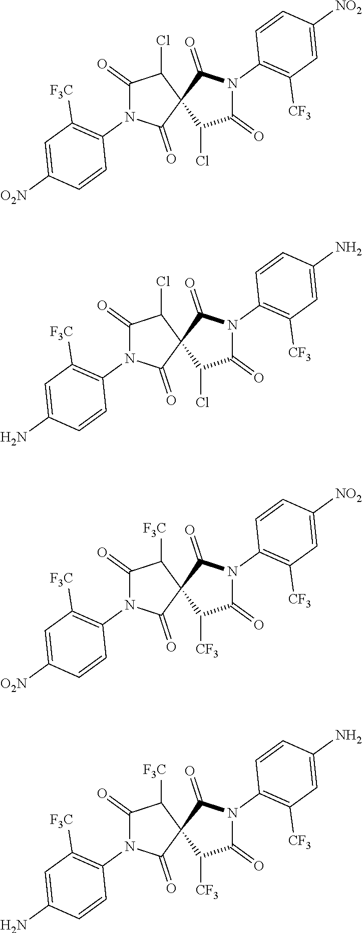

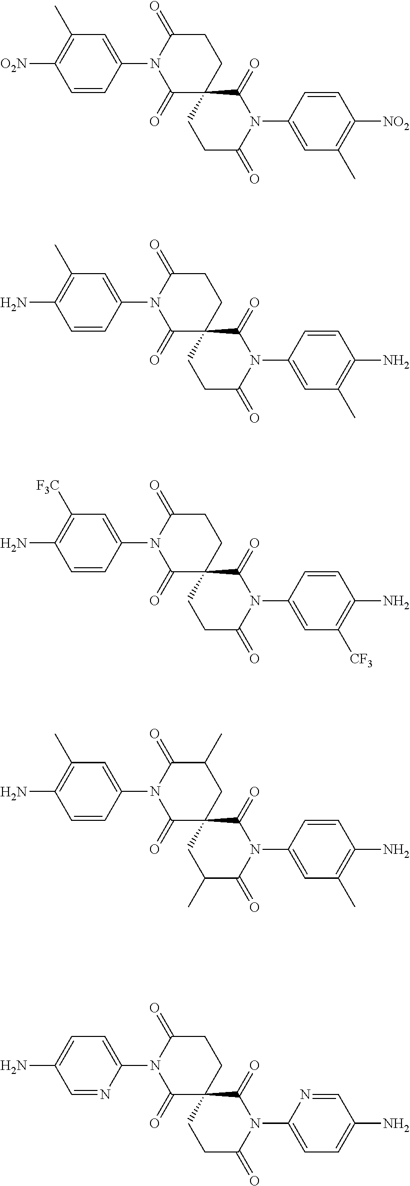

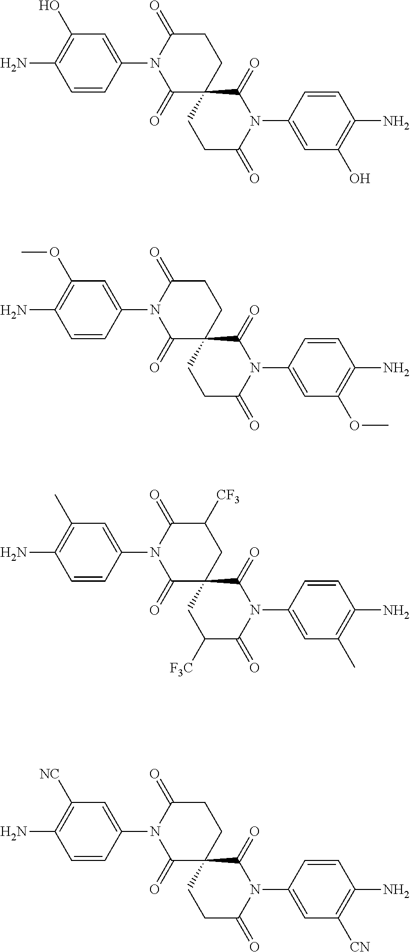

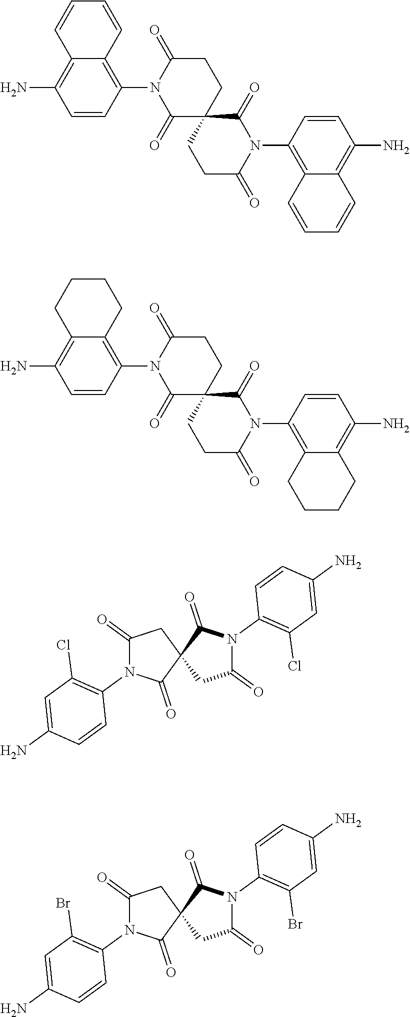

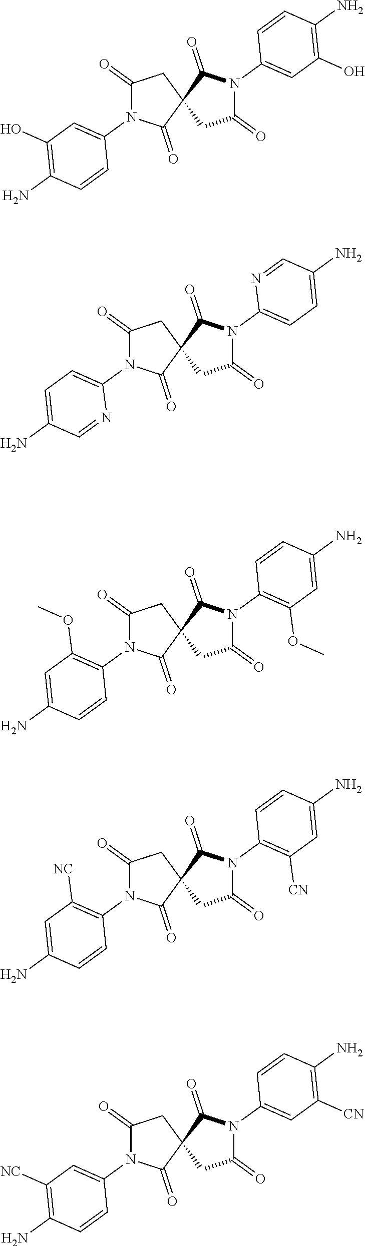

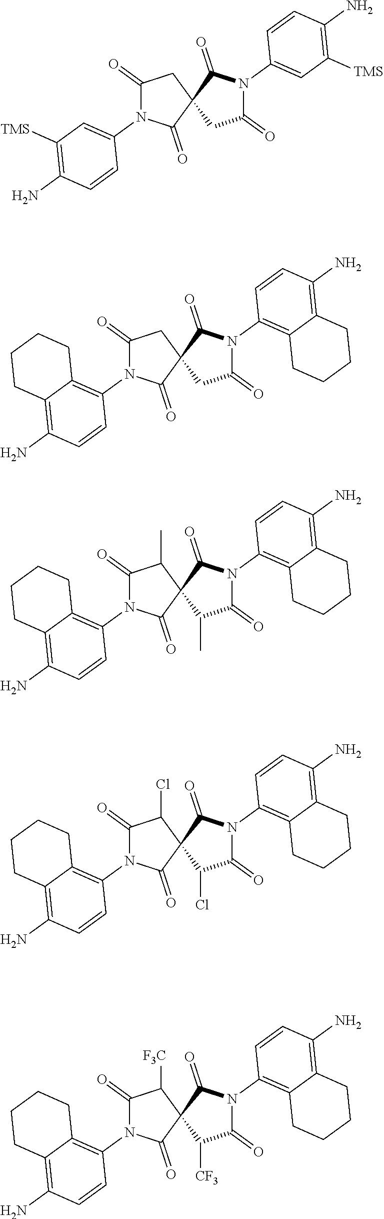

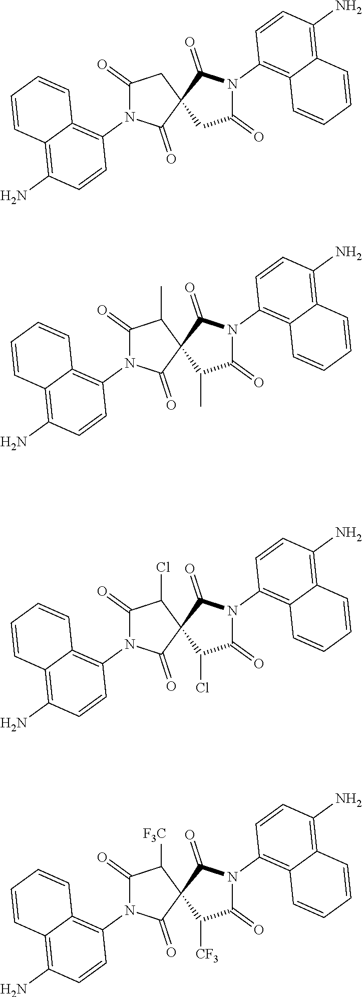

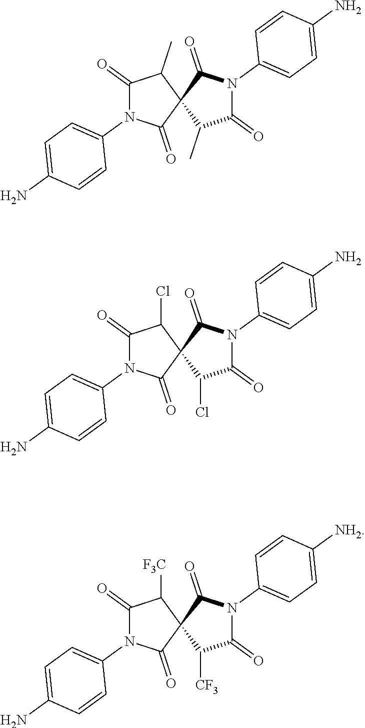

11. A polyimide-based polymer composition comprising a diazaspiro compound represented by the following Chemical Formula 1-1 or 1-2: 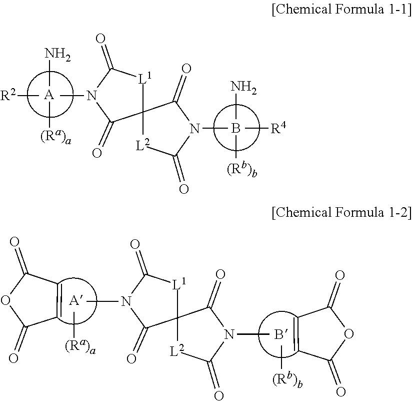
in Chemical Formulas 1-1 and 1-2,
a ring A, a ring B, a ring A′, and a ring B′ are each independently a C6-C20 aromatic ring or a C3-C20 heteroaromatic ring;
L¹ and L² are each independently substituted or unsubstituted hydrocarbylene;
R² and R⁴ are each independently hydrogen, halogen, substituted or unsubstituted hydrocarbyl, —OR^{b11}, —NR^{b12}R^{b13}, —COR^{b14}, —COOR^{b15}, nitro, —SiR^{b16}R^{b17}R^{b18}, or cyano;
R^{a} and R^{b} are each independently halogen, substituted or unsubstituted hydrocarbyl, —OR^{c11}, —NR^{c12}R^{c13}, —COR^{c14}, —COOR^{c15}, nitro, —SiR^{c16}R^{c17}R^{c18}, or cyano, R^{a} and R² may be linked to each other to form a fused ring, and R^{b} and R⁴ may be linked to each other to form a fused ring;
R^{b11} to R^{b18} and R^{c11} to R^{c18} are each independently hydrogen or substituted or unsubstituted hydrocarbyl; and
a and b are each independently an integer of 0 to 2, when a is an integer of 2, each of the R^{a}(s) may be the same as or different from each other, and when b is an integer of 2, each of the R^{b}(s) may be the same as or different from each other.

12. The polyimide-based polymer composition of claim 11, wherein the polyimide-based polymer composition has a structural unit derived from the diazaspiro compound of Chemical Formula 1-1 and a structural unit derived from an acid dianhydride compound represented by the following Chemical Formula D: 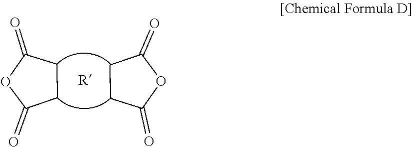
in Chemical Formula D, 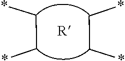
is at least one tetravalent group selected from 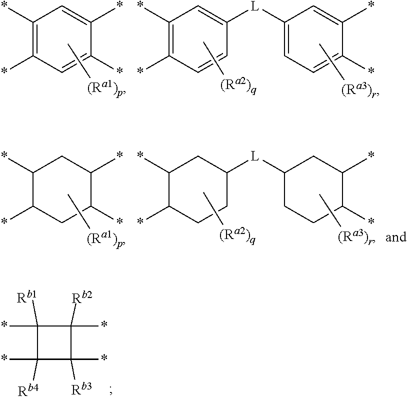
R^{a1}, R^{a2}, and R^{a3} are each independently C1-C10 alkyl or halo C1-C10 alkyl;
L is a single bond, C1-C10 alkylene, —O—, —S—, —CO—, —SO₂—, —SiR^{f1}R^{f2}—, —CO—Ar—CO—, or —O—Ar¹—(X—Ar²)ₛ—O—, and the alkylene may be further substituted with one or more selected from C1-C10 alkyl and halo C1-C10 alkyl;
R^{f1} and R^{f2} are each independently C1-C10 alkyl;
Ar, Ar¹, and Ar² are each independently C6-C20 arylene, and the arylene may be further substituted with one or more selected from C1-C10 alkyl and halo C1-C10 alkyl;
X is O or S;
R^{b1} to R^{b4} are each independently hydrogen, C1-C10 alkyl, or C6-C20 aryl;
p, q, and r are each independently an integer of 0 to 2; and
s is an integer of 0 or 1.

13. The polyimide-based polymer composition of claim 12, wherein the polyimide-based polymer composition has a repeating unit represented by the following Chemical Formula 13: 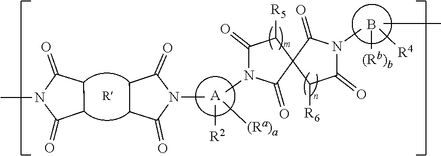
in Chemical Formula 13, 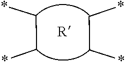
is the same as defined in claim 12;
a ring A and a ring B are each independently a C6-C20 aromatic ring or a C3-C20 heteroaromatic ring;
R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;
R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;
R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;
R^{b17}, R^{b18}, R^{c17}, and R^{c18} are each independently C1-C10 alkyl, C3-C10 cycloalkyl, or C6-C20 aryl;
R⁵ and R⁶ are each independently hydrogen, halogen, C1-C10 alkyl, or halo C1-C10 alkyl;
a and b are each independently an integer of 0 or 1; and
m and n are each independently an integer of 1 to 5.

14. The polyimide-based polymer composition of claim 11, wherein the polyimide-based polymer composition has a structural unit derived from the diazaspiro compound of Chemical Formula 1-2 and a structural unit derived from a diamine compound represented by the following Chemical Formula E: 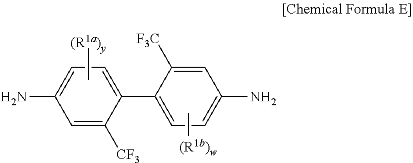
in Chemical Formula E,
R^{1a} and R^{1b} are each independently halogen, C1-C10 alkyl, halo C1-C10 alkyl, C1-C10 alkoxy, or C6-C12 aryl; and
y and w are each independently an integer of 0 to 3.

15. The polyimide-based polymer composition of claim 14, wherein the polyimide-based polymer composition has a repeating unit represented by the following Chemical Formula 14: 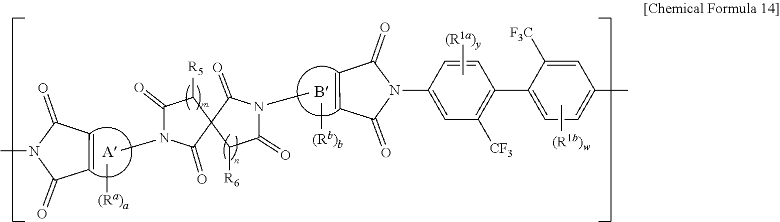
in Chemical Formula 14,
R^{1a}, R^{1b}, y, and w are the same as defined in claim 14;
a ring A′ and a ring B′ are each independently a C6-C20 aromatic ring or a C3-C20 heteroaromatic ring;
R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;
R^{c11}, R^{c12}, R^{c13}, R^{c15}, and R^{c16} are each independently hydrogen, C1-C10 alkyl, C3-C10 cycloalkyl, or C6-C20 aryl;
R^{c17} and R^{c18} are each independently C1-C10 alkyl, C3-C10 cycloalkyl, or C6-C20 aryl;
R⁵ and R⁶ are each independently hydrogen, halogen, C1-C10 alkyl, or halo C1-C10 alkyl;
a and b are each independently an integer of 0 or 1; and
m and n are each independently an integer of 1 to 5.

16. A polyimide film comprising the polyimide-based polymer composition of claim 11.

17. The polyimide film of claim 16, wherein the polyimide film has a repeating unit represented by the following Chemical Formula 13: 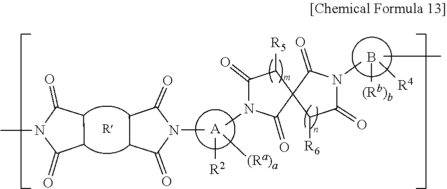
in Chemical Formula 13, 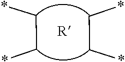
is at least one tetravalent group selected from 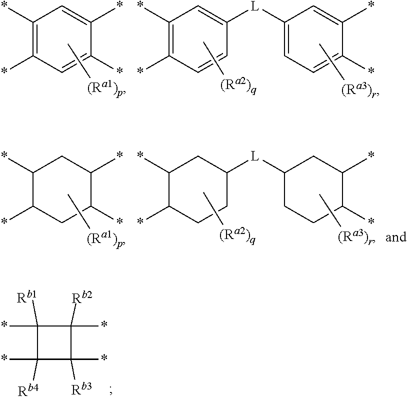
R^{a1}, R^{a2}, and R^{a3} are each independently C1-C10 alkyl or halo C1-C10 alkyl;
L is a single bond, C1-C10 alkylene, —O—, —S—, —CO—, —SO₂—, —SiR^{f1}R^{f2}—, —CO—Ar—CO—, or —O—Ar¹—(X—Ar²)ₛ—O—, and the alkylene may be further substituted with one or more selected from C1-C10 alkyl and halo C1-C10 alkyl;
R^{f1} and R^{f2} are each independently C1-C10 alkyl;
Ar, Ar¹, and Ar² are each independently C6-C20 arylene, and the arylene may be further substituted with one or more selected from C1-C10 alkyl and halo C1-C10 alkyl;
X is O or S;
R^{b1} to R^{b4} are each independently hydrogen, C1-C10 alkyl, or C6-C20 aryl;
a ring A and a ring B are each independently a C6-C20 aromatic ring or a C3-C20 heteroaromatic ring;
R² and R⁴ are each independently hydrogen, halogen, C1-C10 alkyl, halo C1-C10 alkyl, C3-C10 cycloalkyl, C6-C20 aryl, —OR^{b11}, —NR^{b12}R^{b13}, —COOR^{b15}, nitro, —SiR^{b16}R^{b17}R^{b18}, or cyano;
R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano, R^{a} and R² may be linked to each other by C3-C6 alkylene or C3-C6 alkenylene to form a fused ring, and R^{b} and R⁴ may be linked to each other by C3-C6 alkylene or C3-C6 alkenylene to form a fused ring;
R^{b11}, R^{b12}, R^{b13}, R^{b15}, R^{b16}, R^{c11}, R^{c12}, R^{c13}, R^{c15}, and R^{c16} are each independently hydrogen, C1-C10 alkyl, C3-C10 cycloalkyl, or C6-C20 aryl;
R^{b17}, R^{b18}, R^{c17}, and R^{c18} are each independently C1-C10 alkyl, C3-C10 cycloalkyl, or C6-C20 aryl;
R⁵ and R⁶ are each independently hydrogen, halogen, C1-C10 alkyl, or halo C1-C10 alkyl;
a and b are each independently an integer of 0 or 1;
m and n are each independently an integer of 1 to 5;
p, q, and r are each independently an integer of 0 to 2; and
s is an integer of 0 or 1.

18. The polyimide film of claim 16, wherein the polyimide film has a repeating unit represented by the following Chemical Formula 14: 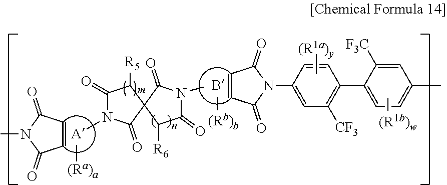
in Chemical Formula 14,
a ring A′ and a ring B′ are each independently a C6-C20 aromatic ring or a C3-C20 heteroaromatic ring;
R^{a} and R^{b} are each independently halogen, C1-C10 alkyl, halo C1-C10 alkyl, C3-C10 cycloalkyl, C6-C20 aryl, —OR^{c11}, —NR^{c12}R^{c13}, —COOR^{c15}, nitro, —SiR^{c16}R^{c17}R^{c18}, or cyano;
R^{c11}, R^{c12}, R^{c13}, R^{c15}, and R^{c16} are each independently hydrogen, C1-C10 alkyl, C3-C10 cycloalkyl, or C6-C20 aryl;
R^{c17} and R^{c18} are each independently C1-C10 alkyl, C3-C10 cycloalkyl, or C6-C20 aryl;
R⁵ and R⁶ are each independently hydrogen, halogen, C1-C10 alkyl, or halo C1-C10 alkyl;
R^{1a} and R^{1b} are each independently halogen, C1-C10 alkyl, halo C1-C10 alkyl, C1-C10 alkoxy, or C6-C12 aryl;
a and b are each independently an integer of 0 or 1;
m and n are each independently an integer of 1 to 5; and
y and w are each independently an integer of 0 to 3.

19. The polyimide film of claim 16, wherein the polyimide film has a yellow index (YI) of 2.0 or less, a light transmittance of 90% or more, a haze of 0.5% or less, and a Young's modulus of 4.5 GPa or less.

20. The polyimide film of claim 16, wherein the polyimide film is used for a device substrate, a display device substrate, an optical film, an integrated circuit (IC) package, an adhesive film, a multi-layer flexible printed circuit (FPC), a tape, a touch panel, or an optical disk protective film.
